(19) United States
(12) Patent Application Publication
(10) Pub. No.: US 2025/0011422 A1
(43) Pub. Date: Jan. 9, 2025

(54) SIGLEC-6 ANTIBODIES, DERIVATIVE COMPOUNDS AND RELATED USES

(71) Applicant: UNIVERSITY OF FLORIDA RESEARCH FOUNDATION, INCORPORATED, Gainesville, FL (US)

(72) Inventors: Christoph RADER, Jupiter, FL (US); Matthew Gerard CYR, Palm Beach Gardens, FL (US)

(73) Assignee: UNIVERSITY OF FLORIDA RESEARCH FOUNDATION, INCORPORATED, Gainesville, FL (US)

(21) Appl. No.: 18/709,790

(22) PCT Filed: Nov. 10, 2022
(86) PCT No.: PCT/US2022/079656

§ 371 Date: May 13, 2024

Related U.S. Application Data
(60) Provisional application No. 63/278,725, filed on Nov. 12, 2021.

(51) Int. Cl. C07K 16/28; A61K 39/00

(52) U.S. Cl. CPC C07K 16/2803; C07K 16/2809; A61K 2039/505; C07K 2317/31; C07K 2317/52; C07K 2317/55; C07K 2317/622; C07K 2317/73; C07K 2317/92; C07K 2317/94

(57) ABSTRACT

The invention provides antibodies, antibody-based fragments or antibody fragments (antigen-binding fragments), as well as derivative molecules such as bispecific T-cell engagers, antibody-drug conjugates (ADCs) and chimeric antigen receptors (CARs), that specifically recognize Siglec-6 and related compositions. Also provided in the invention are methods of using such antibodies in various diagnostic and therapeutic applications.

### CROSS REFERENCE TO RELATED APPLICATIONS

This application is a national stage entry of PCT/US2022/079656 filed Nov. 10, 2022, which claims the benefit of U.S. 63/278,725 filed Nov. 12, 2021, each of which is incorporated by reference in its entirety for all purposes.

### REFERENCE TO AN ELECTRONIC SEQUENCE LISTING

The contents of the electronic sequence listing (614129SEQLST.XML; Size 49,172 bytes; and Date of Creation: May 13, 2024) is herein incorporated by reference in its entirety.

### STATEMENT OF GOVERNMENT INTEREST

This invention was made with government support under Grant Nos. R21 CA229961, RO 1 CA174844 awarded by the National Institutes of Health. The government has certain rights in the invention.

### BACKGROUND OF THE INVENTION

Chronic lymphocytic leukemia (CLL) has the highest incidence of all adult leukemias [SEER Cancer Stat Facts: Chronic Lymphocytic Leukemia. Surveillance, Epidemiology, and End Results Program 2018; seer.cancer.gov/statfactsihtml/clyl.html]. Estimates suggest there is a lifetime risk of 0.6% for the average American. CLL is defined by the expansion of malignant, mature B cells (CD19⁺/CD20⁺/CD5⁺). The 5-year survival rate is favorable, hovering around 80%, due in part to the early detection of indolent, non-aggressive cases. Furthermore, current treatments including anti-CD20 monoclonal antibodies (mAbs) as well as small molecules that target B-cell receptor signaling by inhibiting Bruton's tyrosine kinase (BTK) [Bewarder M. et al., Current Treatment Options in CLL. Cancers (Basel) 2021; 13(10)] or phosphoinositide 3-kinase (PI3K) or antagonizing Bcl-2 have extended remissions in most patients. However, these therapies are not curative; the cost of continuous treatment creates a significant burden; and all have known mechanisms of relapse [Beum P V, et al. J Immunol 2011; 187(6):3438-47; Ahn I E, et al. Blood 2017:129(11):1469-79; Woyach J A, Johnson A J. Blood 2015; 126(4):471-7; Tausch E. et al. Haematologica 2019; 104:e434]. In addition, these treatments lack specificity for leukemic B cells and thus further perpetuate the immune dysfunction in CLL patients, making them more susceptible to infections including among SARS-CoV-2, which was found to be fatal to over one third of CLL patients [Mato A R, et al. Blood 2020; 136(10):1134-43]. Vaccine-induced immunity offers little protection for these patients, as one study of the BNT162b2 mRNA vaccine found that only 16% of patients undergoing CLL treatment produced a detectable antibody response after vaccination and (%% that had received mAbs against CD20, a universal B cell marker, developed anti-SARS-CoV-2 antibodies [Herishanu Y, et al. Blood 2021; 137(23):3165-73]. Therefore the need for CLL-specific therapies that eliminate the leukemia and restore natural immune function is greater than ever.

Allogeneic hematopoietic stem cell transplantation (alloHSCT), a high-risk procedure that is ruled out for most of the CLL patient population, has yielded long remissions or cures in approximately half the patients who have undergone this treatment [Appelbaum J S, Milano F. Curr Hematol Malig Rep 2018; 13(6):484-93]. While most of the alloHSCT-induced graft-versus-leukemia (GVL) response is T cell-mediated, there is evidence of leukemia-targeting antibodies in the sera of treated patients, and these antibodies may be the key for innovative CLL-specific therapies [Wu C J, Ritz J. Adv Immunol 2006, 90:133-73]. Using a phage-display library of a cured CLL patient's alloHSCT antibody repertoire, an antibody was identified JML-1, which binds sialic acid-binding immunoglobulin-like lectin-6 (Siglec-6, also known as OBP 1, CD33L1, and CD327) [Baskar S. et al. Blood 2009; 114(20):4494-502; Chang J, et al. Cancer Immunol Res 2018; 6(9):1008-13].

There is a need for better and more effective antibodies for use in Siglec-6-targeting therapies for treating diseases related to aberrant Siglec-6 expression (e.g., CLL). The present invention is directed to addressing these and other needs.

### SUMMARY OF THE INVENTION

The invention is directed to human antibodies that bind to Siglec-6 with high affinity, derivative molecules containing such antibodies including T cell recruiting bispecific formats. The invention also encompasses diagnostic or therapeutic applications of the antibodies and derivative molecules for CLL, as well as other indications beyond CLL, including but not limited to acute myeloid leukemia, mastocytosis, gestational trophoblastic disease.

In one aspect, the invention provides ab isolated antibody that binds to the same epitope on sialic acid-binding immunoglobulin-like lectin-6 (Siglec-6) as antibody RC-1, RC-2, or ARN-1.

Some antibodies comprise three heavy chain CDRs and three light chain CDRs of antibody RC-1, wherein RC-1 is characterized by a mature heavy chain variable region having an amino acid sequence comprising SEQ ID NO:1 and a mature light chain variable region having an amino acid sequence comprising SEQ ID NO:2: three heavy chain CDRs and three light chain CDRs of antibody RC-2, wherein RC-2 is characterized by a mature heavy chain variable region having an amino acid sequence comprising SEQ ID NO:3 and a mature light chain variable region having an amino acid sequence comprising SEQ ID NO:4: or three heavy chain CDRs and three light chain CDRs of antibody ARN-1, wherein ARN-1 is characterized by a mature heavy chain variable region having an amino acid sequence comprising SEQ ID NO: S and a mature light chain variable region having an amino acid sequence comprising SEQ ID NO:6.

In some antibodies, the CDRs are of a definition selected from the group consisting of Kabat, Chothia. Kabat/Chothia Composite. AbM, Contact, and IMGT. In some antibodies, the mature heavy chain variable region comprises the three Kabat heavy chain CDRs of SEQ ID NOs:7-9 and the mature light chain variable region comprises the three Kabat light chain CDRs of SEQ ID NOs:10-12; the mature heavy chain variable region comprises the three Kabat heavy chain CDRs of SEQ ID NOs: 13-15 and the mature light chain variable region comprises the three Kabat light chain CDRs of SEQ ID NOs:16-18; or the mature heavy chain variable region comprises the three Kabat heavy chain CDRs of SEQ ID NOs: 19-21 and the mature light chain variable region comprises the three Kabat light chain CDRs of SEQ ID NOs:22-24.

Some antibodies comprise a mature heavy chain variable region having an amino acid sequence at least 90% identical to SEQ ID NO:1, and a mature light chain variable region having an amino acid sequence at least 95% identical to SEQ ID NO:2; a mature heavy chain variable region having an amino acid sequence at least 90% identical to SEQ ID NO:3, and a mature light chain variable region having an amino acid sequence at least 95% identical to SEQ ID NO:4; or a mature heavy chain variable region having an amino acid sequence at least 90% identical to SEQ ID NO:5, and a mature light chain variable region having an amino acid sequence at least 90% identical to SEQ ID NO:6.

Some antibodies comprise a mature heavy chain variable region having an amino acid sequence at least 95% identical to SEQ ID NO:1, and a mature light chain variable region having an amino acid sequence at least 95% identical to SEQ ID NO:2; a mature heavy chain variable region having an amino acid sequence at least 95% identical to SEQ ID NO:3, and a mature light chain variable region having an amino acid sequence at least 95% identical to SEQ ID NO:4; or a mature heavy chain variable region having an amino acid sequence at least 95% identical to SEQ ID NO:5, and a mature light chain variable region having an amino acid sequence at least 95% identical to SEQ ID NO:6.

Some antibodies comprise a mature heavy chain variable region having an amino acid sequence of SEQ ID NO:1, and a mature light chain variable region having an amino acid sequence of SEQ ID NO:2; a mature heavy chain variable region having an amino acid sequence of SEQ ID NO:3, and a mature light chain variable region having an amino acid sequence of SEQ ID NO:4; or a mature heavy chain variable region having an amino acid sequence of SEQ ID NO:5, and a mature light chain variable region having an amino acid sequence of SEQ ID NO:6.

Some antibodies are chimeric, humanized, or human. Some antibodies are human.

In some embodiments, the antibody is IgA1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, a dual-affinity re targeting (DART) antibody, or a bivalent antibody.

Some of the antibodies have the mature light chain variable region fused to a light chain constant region and the mature heavy chain variable region fused to a heavy chain constant region. The heavy chain constant region of some antibodies is a mutant form of a natural human heavy chain constant region which has reduced binding to a Fcγ receptor relative to the natural human heavy chain constant region.

In some embodiment, the antibody is a scFv. Some antibodies further comprise a Fc domain that is fused to the C-terminus of the scFv. Some antibodies comprise an amino acid sequence at least 90% identical to SEQ ID NO:25, wherein the mature heavy chain variable region comprises the three Kabat heavy chain CDRs of SEQ ID NOs:7-9 and the mature light chain variable region comprises the three Kabat light chain CDRs of SEQ ID NOs: 10-12.

The antibody can be an antibody fragment. The antibody can be a bispecific antibody. In some embodiments, the bispecific antibody is a T-cell engaging bispecific antibody. The antibody can be an antibody-based binding protein.

Some antibodies are conjugated to a synthetic molecule. In some embodiments, the synthetic molecule is a cytotoxic agent, a label, a therapeutic radioisotope, a diagnostic radioisotope, or a liposome. In some embodiments, the cytotoxic agent is a small molecule weight toxin, a peptide toxin, or a protein toxin.

Some antibodies are linked to at least one cytotoxic agent as an antibody drug conjugate. In some embodiments, the cytotoxic agent is a small molecular weight toxin, a peptide toxin, or a protein toxin, or a radionuclide.

In another aspect, the invention provides a T cell-engaging bispecific antibody (T-biAb), comprising (1) a V_{H} domain and a V_{L} domain of a T cell targeting antibody, and (2) a V_{H} domain and a V_{L} domain of any of the antibodies disclosed herein ((Siglec-6 targeting antibody); wherein the V_{H} domain of the Siglec-6 targeting antibody comprises the three Kabat heavy chain CDRs of SEQ ID NOs:7-9 and the V_{L} domain of the Siglec-6 targeting antibody comprises the three Kabat light chain CDRs of SEQ ID NOs:10-12; wherein the V_{H} domain of the Siglec-6 targeting antibody comprises the three Kabat heavy chain CDRs of SEQ ID NOs: 13-15 and the V_{L} domain of the Siglec-6 targeting antibody comprises the three Kabat light chain CDRs of SEQ ID NOs: 16-18; or wherein the V_{H} domain of the Siglec-6 targeting antibody comprises the three Kabat heavy chain CDRs of SEQ ID NOs:19-21 and the V_{L} domain of the Siglec-6 targeting antibody comprises the three Kabat light chain CDRs of SEQ ID NOs:22-24.

In some T-biAbs, the T cell targeting antibody is an anti-CD3 antibody. In some T-biAbs, the T cell targeting antibody is an anti-CD3 antibody comprising a heavy variable region having an amino acid sequence of SEQ ID NO:34 and light variable region having an amino acid sequence of SEQ ID NO:35. In some T-biAbs, the T cell targeting antibody is an anti-CD3 antibody comprising an amino acid sequence of SEQ ID NO:33. In some T-biAbs, the T cell targeting antibody is an anti-CD3 antibody comprising an amino acid sequence of SEQ ID NO:39.

In some T-biAbs, the V_{H} domain and the V_{L} domain of the T cell targeting antibody forms a first scFv, and the V_{H} domain and the V_{L} domain of the Siglec-6 targeting antibody forms a second scFv. In some T-biAbs, the V_{H} domain of the T cell targeting antibody and the V_{L} domain of the Siglec-6 targeting antibody forms a first tandem V_{H}-V_{L} fragment, and the V_{L} domain of the T cell targeting antibody and the V_{H} domain of the Siglec-6 targeting antibody forms a second tandem V_{H}-V_{L} fragment.

Some T-biAbs further comprise a C-terminal inter-chain disulfide bond to connect the two tandem V_{H}-V_{L} fragments. In some T-biAbs, one or both tandem V_{H}-V_{L} fragments are further fused to an antibody Fc arm in either a V_{H}-V_{L}-Fc format or a V_{L}-V_{H}-Fc format. Some T-biAbs comprise two polypeptide chains each of which comprises a tandem V_{H}-V_{L} fragment that is fused to a Fc arm, wherein the two Fc arms respectively contain knob mutations and hole mutations to allow dimerization of the two polypeptide chains.

In some T-biAbs, the first polypeptide chain comprises a tandem V_{H}-V_{L} fragment of a T cell targeting V_{L} and a Siglec-6 targeting V_{H} that is fused to one Fc arm, and the second polypeptide chain comprises a tandem V_{H}-V_{L} fragment of a Siglec-6 targeting V_{L} and a T cell targeting V_{H} that is fused to the other Fc arm.

In some T-biAbs, (a) the first polypeptide chain comprises an amino acid sequence that is at least 90% identical to SEQ ID NO:26, and a Siglec-6 targeting V_{H} domain comprising the three Kabat heavy chain CDRs of SEQ ID NOs:7-9; and (b) the second polypeptide chain comprises an amino acid sequence that is at least 90% identical to SEQ ID NO:27, and a Siglec-6 targeting V_{L} domain comprising the three Kabat light chain CDRs of SEQ ID NOs: 10-12.

In some T-biAbs, the first polypeptide chain comprises a tandem V_{H}-V_{L} fragment of a Siglec-6 targeting V_{L} and a T cell targeting V_{H} that is fused to one Fc arm, and the second polypeptide chain comprises a tandem V_{H}-V_{L} fragment of a T cell targeting V_{L} and a Siglec-6 targeting V_{H} that is fused to the other Fc arm.

In some T-biAbs, where (a) the first polypeptide chain comprises an amino acid sequence that is at least 90% identical to SEQ ID NO:28, and a Siglec-6 targeting V_{L} domain comprising the three Kabat light chain CDRs of SEQ ID NOs:10-12; and (b) the second polypeptide chain comprises an amino acid sequence that is at least 90% identical to SEQ ID NO:29, and a Siglec-6 targeting V_{H} domain comprising the three Kabat heavy chain CDRs of SEQ ID NOs:7-9.

Some T-biAbs comprise three polypeptide chains, wherein (a) the first polypeptide chain comprises a first tandem V_{H}-V_{L} fragment having a first C-terminal coil motif that is fused to a first Fc arm, (b) the second polypeptide chain comprises the other tandem V_{H}-V_{L} fragment having a second C-terminal coil motif, and (c) the third polypeptide chain comprises a second Fc arm; wherein one of the Fc arms contains knob mutations, and the other Fc arm contains hole mutations; and wherein one of the C-terminal coil motif is a E-coil, and the other C-terminal coil motif is an oppositely charged K-coil.

In some T-biAbs, (a) the first tandem V_{H}-V_{L} fragment comprises a Siglec-6 targeting V_{L} and a T cell targeting V_{H}, and the second tandem V_{H}-V_{L} fragment comprises a T cell targeting V_{L} and a Siglec-6 targeting V_{H}, or (b) the first tandem V_{H}-V_{L} fragment comprises a T cell targeting V_{L} and a Siglec-6 targeting V_{H}, and the second tandem V_{H}-V_{L} fragment comprises a Siglec-6 targeting V_{L} and a T cell targeting V_{H}.

In some T-biAbs, (a) the first polypeptide chain comprises (i) a Siglec-6 targeting V_{L} domain comprising the three Kabat light chain CDRs of SEQ ID NOs:10-12 and (ii) an overall amino acid sequence that is at least 90% identical to SEQ ID NO:30; (b) the second polypeptide chain comprises (i) a Siglec-6 targeting V_{H} domain comprising the three Kabat heavy chain CDRs of SEQ ID NOs:7-9 and (ii) an overall amino acid sequence that is at least 90°% identical to SEQ ID NO:31, and (c) the third polypeptide chain comprises an amino acid sequence that is at least 90% identical to SEQ ID NO:32.

In another aspect, the invention provides a chimeric antigen receptor (CAR), comprising an extracellular domain, a transmembrane domain, and an intracellular signaling domain, wherein the extracellular domain comprises any of the antibodies disclosed herein. In another aspect, the invention provides an effector cell expressing any of the chimeric antigen receptors disclosed herein. In some embodiments, the effector cell is a T cell or a natural killer (NK) cell.

In another aspect, the invention provides a pharmaceutical composition comprising a therapeutically effective amount of any of the antibodies or the T cell-engaging bispecific antibodies disclosed herein and a pharmaceutically acceptable carrier.

In another aspect, the invention provides a polynucleotide encoding a heavy chain variable region and/or a light chain variable region of any of the antibodies disclosed herein. In another aspect, the invention provides a vector comprising such a polynucleotide. In another aspect, the invention provides a host cell harboring such a vector.

In another aspect, the invention provides a method of treating a disease or disorder associated with aberrant Siglec-6 expression in a subject, comprising administering the pharmaceutical composition disclosed herein to a subject in need thereof.

In some methods, the disease or disorder is a tumor, a leukemia, or a mast cell disorder. In some methods, the disease or disorder is a chronic or an acute leukemia. In some methods, the disease or disorder is chronic lymphocytic leukemia (CLL). In some methods, the disease or disorder is a tumor containing Siglec-6 expressing myeloid-derived suppressor cells (MDSCs). In some methods, the disease or disorder is an autoimmune disease or an inflammatory disease. In some methods, the autoimmune disease or the inflammatory disease is multiple sclerosis, rheumatoid arthritis, systemic lupus erythematosus, type 2 diabetes mellitus, cytokine release syndrome, graft-versus-host-disease, or HIV-associated immunopathogenesis.

A further understanding of the nature and advantages of the present invention may be realized by reference to the remaining portions of the specification and claims.

### DESCRIPTION OF THE DRAWINGS

FIG. 1 shows heavy chain variable and light chain variable region amino acid sequences of 3 human anti-Siglec-6 clones, RC-1 (SEQ ID NOs:1 and 2), RC-2 (SEQ ID NOs:3 and 4), and ARN-1 (SEQ ID NOs:5 and 6). Kabat-defined CDRs of each antibody chains are underlined (SEQ ID NOs:7-24, respectively).

FIG. 2 shows characteristics of Siglec-6-targeting antibody clones identified from post-alloHSCT library. (a) Comparison of post-alloHSCT antibody clones including variable heavy (V_{H}) and light chain domain (V_{L}) amino acid sequence identities, IMGT-defined complementarity determining region 3 (CDR3) alignments depicting unique residues in boldface, and affinity data from surface plasmon resonance (SPR) analysis of Fab binding to Siglec-6-Fc fusion protein. Sequences of IMGT HCDR3 and IMGT LCDR3 of antibodies JML-1 (SEQ ID NOs:40 and 41), RC-1 (SEQ ID NOs:42 and 12) and RC-2 (SEQ ID NO:40 and 18) are aligned. (b) ELISA consisting of directly coating human or macaque (85% amino acid sequence identity within the ectodomain) Siglec-Fc fusion proteins, incubating with Fabs selected from the post-alloHSCT library, and detecting with anti-human Fab or anti-human Fc (positive control for antigen coating). The post-alloHSCT Fabs are all specific for human Siglec-6. (c) Flow cytometry histograms (left) and quantification (right) demonstrating that JML-1 Fab can block the binding of biotinylated RC-1 (RC-1-bio) Fab (40 nM) by competing for Siglec-6 on the surface of U937 cells. Statistics (n=3) were calculated using a student's t-test, ****p<0.0001. (d) Homology model of Siglec-6 (V-type (V) and C2-type I Ig-like (C2i) domains, 27-236), shaded according to the differential deuterium uptake in the presence of RC-1 Fab as determined by hydrogen-deuterium exchange (HDX) mass spectrometry (MS) (HDX-MS). Negative differential D₂O values represent decreased solvent exposure in the presence of Fab. Differential deuterium update is quantified in FIG. 5(c). Black regions were not observed (areas that are boxed in FIG. 5(d)). (e) Homology model of Siglec-6 illustrating the peptides that were individually substituted with corresponding CD33 sequences (bolded sequences in FIG. 5(d)), and the C2-type I Ig-like domain that was replaced entirely for epitope mapping (sequence depicted in italicized boldface in FIG. 5(d); (f) Epitope mapping ELISA with Siglec-6×CD33 chimeric Fc-fusion proteins indicating the dependence of the mutated regions for binding with human Fabs but not for the commercial mouse mAb or sheep pAb to human Siglec-6 (hS6).

FIG. 3 shows Siglec-6 post-alloHSCT phage panning outputs. Enrichment was observed in the phage output titer through subsequent panning rounds (top). Fab-phage ELISA data (bottom) demonstrating that each panning generated polyclonal phage that bind to Siglec-6 in the presence or absence of JML-1 Fab. Abs=absorbance.

FIG. 4 shows Fab kinetics for Siglec-6. SPR sensorgrams of soluble Fabs RC-1 (a) and RC-2 (b) binding to Fc-Siglec-6 (5 μg/mL). (c) Table of SPR data. (d) SPR sensorgram demonstrating that there is no additive effect of RC-1 over JML-1 alone (1 μM each, saturating) validating that the two antibody clones target overlapping epitopes and cannot bind simultaneously to Fc-Siglec-6 (0.5 μg/mL).

FIG. 5 shows epitope mapping by HDX-MS. (a) Homology model of the two N-terminal Siglec-6 V-type and C2-type I Ig-like domains overlayed with shading to indicate the level of deuterium uptake of the ectodomain fragment in the absence of Fab. Differential deuterium update from 5(a) is quantified in FIG. 5(c). (b) Deuterium uptake versus time plots for two selected peptides, with the results from the Siglec-6 ectodomain fragment alone (solid diamond) and its complex with RC-1 Fab (unfilled diamond). Peptide on top panel of 5(b) is SEQ ID NO:44, and peptide on bottom panel of 5(b) is SEQ ID NO:45. These plots are representative of peptides that show reduced (left) or unchanged (right) uptake in the presence of Fab. (c) Sequence map of the Siglec-6 construct (SEQ ID NO:37) used for HDX-MS (note: N- and C-terminal expression tags differ from the WT sequence) showing differential deuterium uptake data+/−Fab. Peptides are shown as bars below the sequence, with differential deuterium uptake values for each peptide shown next to or below respective bar; (d) Sequence alignment of human Siglec-6 (V-type and C2-type I Ig-like domains, 27-235; SEQ ID NO:46), rhesus macaque Siglec-6 (UniProt accession AOAID5QH63, 16-226; SEQ ID NO:47) and human CD33 (21-232; SEQ ID NO:48), with dots indicating identity to human Siglec-6. The underlined residues were part of peptides that showed differential deuterium uptake in the presence of Fab, while the boxed regions indicate that the peptides were not observed in the mass spectrometry data. Bolded sequences (QERRFQLEGP, amino acid residues 1-10 of SEQ ID NO:46: RLPTTLPASYYGY, amino acid residues 26-38 of SEQ ID NO:46; LKSKWM, amino acid residues 97-102 of SEQ ID NO:46; (IYTSSK, amino acid residues 105-110 of SEQ ID NO:46) indicate the Siglec-6 regions required for Fab binding in ELISA, while sequences in italicized boldface (amino acid residues 121-209 of SEQ ID NO:46) indicate regions that are not required for binding in ELISA. The numbering shown in this alignment in FIG. 5(d) is based on full-length, canonical isoforms found in the UniProtKB database.

FIG. 6 shows ARN-1 human Fab clone characteristics. (a) Comparison of human anti-Siglec-6 antibody clones including the library of origin, germline gene usage, IGHD length, IMGT-defined complementarity determining region 3 (CDR3) alignments, affinity data from SPR analysis of monovalent Fab binding to Siglec-6-Fc fusion protein, and divergence from germline sequences. Sequences of IMGT HCDR3 and IMGT LCDR3 of antibodies JML-1 (SEQ ID NOs:40 and 41), RC-1 (SEQ ID NOs:42 and 12). RC-2 (SEQ ID NO:40 and 18), and ARN-1 (SEQ ID NO:43 and 21) are shown. Underlined regions indicate unique sequence features. (b) ELISA consisting of directly coating human or macaque Siglec-Fc fusion proteins, incubating with Fabs, and detection with anti-human Fab or anti human Fc (positive control for antigen coating). ARN-1 has a similar specificity for Siglec-6 in comparison to the post-alloHSCT Fabs. (c) Flow cytometry staining of U937 cells with ARN-1, RC-1, JML-1, and isotope control Fabs, followed by PE-anti-human Fab detection. MFI=mean fluorescence intensity (d) Flow cytometry histograms demonstrating that ARN-1 Fab can block the binding of biotinylated RC-1 (RC-1-bio) Fab (4 nM) by competing for Siglec-6 on the surface of U937 cells.

FIG. 7 shows Siglec-6 is expressed on primary CLL cells. (a) Quantification of Siglec-6 expression (mouse mAb clone 767329) on primary, CLL cells (live, CD20⁺ CD5⁺, n=6). ABC denotes antibody binding capacity=1 to 2 copies of the receptor on the cell surface per antibody bound. (b) Flow cytometry staining of primary CLL cells using mouse mAb 767329. Representative histograms (left) and quantified mean fluorescence intensity (MF, right) demonstrate Siglec-6 is expressed on CLL cells (live, CD20⁺ CD5⁺ thin line) yet absent on T cells (CD3⁺ CD5⁺, thick line) from individual CLL patients (n=16). Statistics were calculated using Wilcoxon matched-pairs signed rank test **** p<0.0001. (c) Binding of human anti-Siglec-6 clone JML-1 (biotinylated IgG1, left) or RC-1 (biotinylated Fab) to CLL and healthy donor PBMC. Each point represents an individual patient. Since staining was done on different days, the data in each experiment were normalized to staining with an isotype control antibody for each patient and the dotted line marks the level of isotype binding. Statistics were calculated using an unpaired t test with Welch's correction, * p<0.05. (d) Quantification of Siglec-6 levels on CLL-derived cell lines as ABC using mouse mAb 767329 or isotype control and a bead calibration standard. MEC1-hS6 refers to a clone of MEC1 that had been stably transfected to overexpress Siglec-6.

FIG. 8 shows scFv-Fc T-biAbs mediate specific T cell activation and CLL lysis (a) T-biAbs were designed with scFv domains fused to human IgG1 Fc with knobs-into-holes mutations to facilitate heterodimerization of Siglec-6 and CD3 binding arms. (b) Siglec-6-negative CLL cell line MEC1 (left), Siglec-6-positive MEC1-002 (middle), and human T cells (right) were stained with 10 μg/mL of scFv-Fc T-biAb and an anti-human Fc secondary antibody to validate bispecificity for Siglec-6 (or CD19 as positive control) and CD3. (c) A Jurkat-Lucia NFAT reporter cell line was cultured overnight with scFv-Fc T-biAbs and MEC1 (left) or MEC1-002 (right) cells to determine T cell activation in the absence or presence of target expression. (d) MEC1 (left) and MEC1-Siglec-6 (hS6) transgenic (right) cell lines were co-cultured with human T cells and a titration of the indicated T biAbs. Following overnight incubation, cell lysis was assessed by intracellular luciferase activity. (e) The levels of type I cytokines in the cell lysis assay supernatants were determined by ELISA, and data were normalized to the CD19/V9 positive control. Statistics were calculated using an unpaired t-test, n=3, ** p<0.05, ** p<0.01, ** p<0.001.

FIG. 9 shows scFv-Fc purification and validation. After an initial round of Protein A affinity chromatography purification, size exclusion chromatography (SEC) was used to purify JML-1/V9 scFv-Fc (a) and RC-1/V9 scFv-Fc (b). The resulting chromatograms are shown and the major 105 kDa peak occurring around 12 mL for each, was isolated and verified to be free of aggregates by reducing (r) and non-reducing (nr) SDS-PAGE and Coomassie Blue staining (c).

FIG. 10 shows T cell activation markers and cytokines following T-biAb treatment. MEC1-fLuc-Siglec-6 transgenic cell lines (MEC1 hS6 cells) were co-cultured with human T cells at a 1:1 E:T ratio and a titration of the indicated T-biAbs. Following overnight incubation, T cells were stained for activation markers CD69 and CD25. Shown are representative scatterplots from cells treated with 312 pM scFv-Fc (a) and 5 pM of scFv-Fc or DART-Fc (c). (b) MEC1-002 (hS6^{l0}) cells were cultured overnight with T cells at a 1:1 E:T ratio in the presence of 800 pM of T-biAb, and the levels of type I cytokines in the co-culture supernatants were determined by ELISA.

FIG. 11 shows DART-Fc T-biAbs elicit more potent activation and lysis than scFv-Fc. (a) Cartoon depiction of Siglec-6×CD3 DART-Fc constructs. In all 3 constructs, Fc dimerization was mediated by knobs-into-holes mutations. Two symmetric DART-Fc (sDART) constructs were made, alternating the positioning of V_{L} and V_{H} for each clone. sDART-Fc T-biAbs are labeled in terms of the V_{L} clone at the N-terminus of the holes-Fc chain, RC-1/V9 sDART-Fc and V9/RC-1 sDART-Fc. The asymmetric DART-Fc (aDART) construct consists of 3 polypeptide chains and was made by inserting oppositely charged coiled-coil domains at the C-terminus of each V_{H} domain to stabilize the interaction between the free V_{L}-V_{H} chain and the two chains with dimerized Fc domains. (b) Siglec-6-negative CLL cell line MEC1, Siglec-6-positive MEC1-hS6, and human T cells were stained with 10 nM of DART-Fc T-biAb and an anti-human Fc secondary antibody to validate T-biAb specificity for Siglec-6 (or CD19 as positive control) and CD3. (c) A Jurkat-Lucia NFAT reporter cell line was cultured overnight with scFv-Fc T-biAbs and MEC1, or MEC1-002 CLL cell lines (E:T=1:1) to determine T cell activation in the absence or presence of target expression. (d) MEC1 and MEC1-hS6 cell lines were co-cultured with human T cells (E:T=1:1) and a titration of the indicated T-biAbs. Following overnight incubation, cell lysis was assessed by intracellular luciferase activity. (e) Cytokine levels in the culture media from the overnight cytotoxicity assay were determined by ELISA, and data were normalized to the CD19/V9 positive control at the high dose (250 pM). Statistics were calculated using an unpaired t-test, n=3, ** p<0.05, ** p<0.01, *** p<0.001, ****<0.0001.

FIG. 12 shows DART-Fc purification and validation. (a) Reducing (r) and non-reducing (nr) Coomassie Blue-stained SDS-PAGE gel of DARTs after an initial round of MabSelect SuRe Protein A affinity chromatography purification revealed the expected size of the monomers in the non-reducing lanes and the dimer (sDART-Fc) or trimer (aDART-Fc) in the reducing lanes. (b) To eliminate the lower molecular weight bands observed in the RC-1/V9 aDART-Fc, selective heterodimer purification was conducted by gradient elution from the POROS mAb Capture A column which allows for separation of Fc-Fc* heterodimers and Fc-Fc homodimers on the basis of differential affinities in the presence of 500 mM calcium chloride, a chaotropic salt. (c) Analytical SEC area under the curve (AUC) analysis revealed >96% of the protein in the major peak corresponding to the 107.9 kDa RC-1/V9 aDART-Fc trimer. (d) SDS-PAGE verified the improved purity of the final RC-1/V9 aDART-Fc product.

FIG. 13 shows RC-1/V9 aDART-Fc efficacy on low expression MEC1-002 cells and at low E:T ratios. (a) Cytotoxicity of an overnight co-culture at a 1:1 E:T ratio with Siglec-6-negative (MEC1), -low (MEC1-002), or -high (MEC1-hS6) expression cell lines, in the presence of titrated T-biAb. (b, c) Cytotoxicity assays consisting of a 48 h co-culture of 1 nM T-biAb, 50,000 MEC1-002 or MEC1-hS6 target cells, and a titration of T cells from 50,000 down to 1,500 cells/sample. (c) Bar graph comparing specific lysis at 1:10 and 1:30 E:T ratios for CD19/V9 scFv-Fc or RC-1/V9 aDART-Fc demonstrating that specific lysis is still achieved at 1:30 E:T ratio even with relatively low Siglec-6 expression on MEC1-002 cells. Siglec-6 targeting scFv-Fc and aDART-Fc were titrated against target-negative (MEC1, d) or target-low (MEC1-002, e) cells with a 1:30 E:T ratio of T cells over a 48 h co-culture and the EC₅₀ values for RC-1/V9 aDART-Fc were more than 10-fold lower than that of the RC-1/V9 scFv-Fc and almost 3-fold lower than the CD19/V9 scFv-Fc.

FIG. 14 shows RC-1/V9 aDART-Fc mediates lysis of primary CLL B cells. (a) Treatment-naive CLL PBMC (n=7) were cultured with 6 nM T-biAbs for 11 days and analyzed by flow cytometry to determine specific lysis of CLL cells by autologous T cells at endogenous E:T ratios. The activity of each T-biAb was compared to the corresponding non-targeting (NT) control of the same construct. (b) CLL PBMC were cultured as in (a) but for 13 days on a larger set of patients (n=24). (c) To assess lysis by allogeneic (allo-) T cells, healthy donor (HD) PBMC were co-cultured with CLL PBMC (n=9) at an E:T ratio of 1:1 and specific lysis was assessed on day 7. (d) Comparing T-biAb-mediated specific lysis of CLL cells from individual patients (n=8) by autologous (auto-) or allo-T cells after 7 days of ex vivo culture. (e) Cell lysis at day 11 correlated with Siglec-6 expression levels as determined by Spearman's correlation analysis. All pairwise comparisons were made using the Wilcoxon matched-pairs signed-rank test, *p<0.05, ** p<0.01, *** p<0.001.

FIG. 15 shows targeting Siglec-6 on primary CLL cells. (a) CLL PBMC were co-cultured with RC-1/V9 aDART-Fc (6 nM) for 3 to 13 days and analyzed by flow cytometry to determine specific lysis of CLL cells. Statistics were calculated to compare different time points using a Wilcoxon matched-pairs signed rank test. (b) Siglec-6 expression and E:T ratio were established at baseline and these variables correlated by Spearman's correlation analysis. (c) Specific lysis data from day 13, separating patients based on Siglec-6 expression. Statistics were calculated using the Mann-Whitney test.

FIG. 16 shows CLL-derived T cell expansion and activation. (a) CLL PBMC were cultured with RC-1/V9 aDART-Fc or CD19/V9 scFv-Fc (6 nM) for 9 days and analyzed by flow cytometry to quantify the number of CD4+ and CD8+ T cells present. (b) Also at day 9, the fraction of each subset expressing both CD25 and CD69 activation markers was quantified. Statistics were calculated using the Wilcoxon matched-pairs signed rank test.

FIG. 17 shows comparing biAb constructs at the biophysical and cellular level. (a) MEC1-hS6 (top) or primary human T cells (bottom) were stained with a titration of T-biAbs, and binding was detected by the addition of an Alexa Fluor 647-conjugated anti-human Fc secondary antibody. (b) RC-1/V9 biAbs were immobilized to a CMS Biacore chip via the Fc domain, soluble Siglec-6 or CD3ε/δ dimer was injected, and SPR sensorgrams were used to determine kₒₙ, k_{off}, and K_{d}. (c) Far Red-labeled MEC1-hS6 cells (1×10⁶ cells) were bound with 50 nM T-biAb, washed, and co-cultured with carboxyfluorescein succinimidyl ester (CFSE)-labeled Jurkat cells (1×10⁵ cells) for 1 h at 37° C. followed by fixation, permeabilization, and staining with Cy3-conjugated anti-human Fc and DAPI. Samples were processed on an Amnis lmageStreamx MK 11 (Luminex) instrument and analyzed by IDEAS 6.3 software to identify CFSE+/Far Red+images (top) and T-biAb+synapses (bottom). (d) The percentage of T cells forming synapses was quantified for three independent experiments and data were compared using a paired t-test. (e) Internuclear distance was calculated by measuring the distance between synapse-positive cells in the DAPI channel. The mean and standard error of the mean for approximately 1,000 events per sample are plotted from one representative experiment and statistics were calculated using an unpaired t-test, *p<0.05, **p<0.01.

FIG. 18 shows RC-1/V9 aDART-Fc clears CLL cells in a CDX model. NSG mice were inoculated i.v. with 2×10⁶ MEC1-fLuc-hS6 cells and on day 7 were treated with 3×10⁶ T cells i.v., followed by twice weekly treatment with T-biAb (0.5 mg/kg or 0.05 mg/kg) for 2 weeks. On day 13, mice receive a second injection of only 1×10 T cells. (a) Bioluminescence images and (b) quantification (one line per mouse) showed complete clearance of CLL cells in mice treated with T cells and CD19/V9 or RC-1/V9 T-biAbs, but not the non-targeting (NT/V9) T-biAb. Statistics were calculated comparing both doses of RC-1/V9 with NT/V9 using an unpaired t-test with Welch's correction, n=5 mice per group (**p<0.01, ***p<0.001, #=image saturation). (c) Weight loss was not observed in any animals over the course of treatment. (d) Kaplan-Meier survival analysis showed that RC-1/V9 (0.05 mg/kg) and CD19/V9 (0.5 mg/kg) both improved survival over the NT/V9 control (0.5 mg/kg). Statistics were calculated using the Mantel-Cox log-rank analysis, hazard ratio=9.95 (*p<0.05).

FIG. 19 shows Siglec-6 in BTKi-treated CLL patients. Siglec-6 expression (a-b) for CLL patients before and after beginning BTKi therapy (n=7). (c) E:T ratio for CLL patients before and after beginning BTKi therapy. Samples from these BTKi-treated patients were also tested for cytotoxicity elicited by 6 nM T-biAb in ex vivo cultures at day 3 (d, n=10) and day 11 (e, n=8). Please note that BTKi treatment pressure was not applied during ex vivo culture. All paired statistics are based on Wilcoxon analyses.

FIG. 20 shows Siglec-6 T-biAb treatment of healthy donor PBMC. (a) Healthy donor PBMC (n=6) were cultured with T-biAbs, using conditions identical to the CLL lysis assays, and specific healthy B cell lysis=(Viability_{vehicle}−Viability_{treated})/Viability_{vehicle} was assessed on day 11. Statistics were calculated using the Wilcoxon test. (b) Siglec-6 expression and T cell to B cell (E:T) ratios in for healthy donor (HD) PBMC. (c) The fraction of viable healthy B cells expressing Siglec-6 after 11-day ex vivo culture in the presence or absence of T-biAbs. (Note: CD19/V9 data are not shown, as too few viable B cells were remaining to conduct reliable statistical analyses). Statistics were calculated using the Wilcoxon test (n=8). (d) The fraction of healthy B cells expressing Siglec-6 at baseline (day 0) and day 11. Statistics were calculated using the Wilcoxon test (n=8).

### BRIEF DESCRIPTION OF THE SEQUENCES

SEQ ID NO:1 sets forth the amino acid sequence of V_{H} region of Siglec-6 antibody RC-1 clone (SEQ ID NO:1):

SEQ ID NO:2 sets forth the amino acid sequence of V_{κ} region of Siglec-6 antibody RC-1 clone

SEQ ID NO:3 sets forth the amino acid sequence of V_{H region} of Siglec-6 antibody RC-2 clone

SEQ ID NO:4 sets forth the amino acid sequence of V_{κ region} of Siglec-6 antibody RC-2 clone

SEQ ID NO:5 sets forth the amino acid sequence of V_{H} region of Siglec-6 antibody ARN-1 clone (

SEQ ID NO:6 sets forth the amino acid sequence of V_{κ} region of Siglec-6 antibody ARN-1 clone

SEQ ID NO:7 sets forth the amino acid sequence of Kabat HCDR1 of Siglec-6 antibody RC-1 clone

SEQ ID NO:8 sets forth the amino acid sequence of sets forth the amino acid sequence of Kabat HCDR2 of Siglec-6 antibody RC-1 clone

SEQ ID NO:9 sets forth the amino acid sequence of Kabat HCDR3 of Siglec-6 antibody RC-1 clone

SEQ ID NO: 10 sets forth the amino acid sequence of Kabat LCDR1 of Siglec-6 antibody RC-1 clone

SEQ ID NO:11 sets forth the amino acid sequence of Kabat LCDR2 of Siglec-6 antibody RC-1 clone

SEQ ID NO: 12 sets forth the amino acid sequence of Kabat LCDR3 of Siglec-6 antibody RC-1 clone

SEQ ID NO: 13 sets forth the amino acid sequence of Kabat HCDR1 of Siglec-6 antibody RC-2 clone

SEQ ID NO: 14 sets forth the amino acid sequence of Kabat HCDR2 of Siglec-6 antibody RC-2 clone

SEQ ID NO: 15 sets forth the amino acid sequence of Kabat HCDR3 of Siglec-6 antibody RC-2 clone

SEQ ID NO: 16 sets forth the amino acid sequence of Kabat LCDR1 of Siglec-6 antibody RC-2 clone

SEQ ID NO: 17 sets forth the amino acid sequence of Kabat LCDR2 of Siglec-6 antibody RC-2 clone

SEQ ID NO: 18 sets forth the amino acid sequence of Kabat LCDR3 of Siglec-6 antibody RC-2 clone

SEQ ID NO: 19 sets forth the amino acid sequence of Kabat HCDR1 of Siglec-6 antibody ARN-1 clone

SEQ ID NO:20 sets forth the amino acid sequence of Kabat HCDR2 of Siglec-6 antibody ARN-1 clone

SEQ ID NO:21 sets forth the amino acid sequence of Kabat HCDR3 of Siglec-6 antibody ARN-1 clone

SEQ ID NO:22 sets forth the amino acid sequence of Kabat LCDR1 of Siglec-6 antibody ARN-1 clone

SEQ ID NO:23 sets forth the amino acid sequence of Kabat LCDR2 of Siglec-6 antibody ARN-1 clone

SEQ ID NO:24 sets forth the amino acid sequence of Kabat LCDR3 of Siglec-6 antibody ARN-1 clone

SEQ ID NO:25 sets forth the amino acid sequence of Siglec-6 scFv/Fc fusion: RC-1 scFv-Fc with aglycosylation N297A and “knob” mutations (S354C, T366W) (SEQ ID NO:25):

SEQ ID NO:26 sets forth the amino acid sequence of first chain of T-biAb V91RC-1 sDART-Fc

SEQ ID NO:27 sets forth the amino acid sequence of Second chain of T-biAb V9/RC-1 sDART-Fc (SEQ ID NO:27)

SEQ ID NO:28 sets forth the amino acid sequence of first chain of T-biAb RC-1/V9 sDART-Fc

SEQ ID NO:29 sets forth the amino acid sequence of Second chain of T-biAb RC-1/V9 sDART-Fc

SEQ ID NO:30 sets forth the amino acid sequence of first chain of T-biAb RC-1/V9 aDART-Fc

SEQ ID NO:31 sets forth the amino acid sequence of Second chain of T-biAb RC-1/V9 aDART-Fc (SEQ ID NO:31):

SEQ ID NO:32 sets forth the amino acid sequence of third chain of T-biAb RC-1/V9 aDART-Fc

SEQ ID NO:33 sets forth the amino acid sequence of anti-CD3 scFv (V9)

SEQ ID NO:34 sets forth the amino acid sequence of VΠ region of anti-CD3 scFv (V9)

SEQ ID NO:35 sets forth the amino acid sequence of V_{L} region of anti-CD3 scFv (V9)

SEQ ID NO:36 sets forth the amino acid sequence of linker residues of anti-CD3 scFv (V9)

SEQ ID NO:37 sets forth the amino acid sequence of Siglec-6 VC₂₈₋₂₃₅-His6 in pHL-sec

SEQ ID NO:38 sets forth the amino acid sequence of JML-1 scFv-Fc with aglycosylation N297A and “knob” mutations (S354C, T366W)

SEQ ID NO:38 Sequence of JML-1 scFv-Fc with aglycosylation N297A and “knob” mutations (S354C, T366W)

SEQ ID NO:39 sets forth the amino acid sequence of shared anti-CD3 (V9 clone) with Fc with aglycosylation N297A and “hole” mutations (Y349C, T366S, L368A, and Y407)

SEQ ID NO:40 sets forth the amino acid sequence of JML-1 and RC-2 IMGT HCDR3

SEQ ID NO:41 sets forth the amino acid sequence of JML-1 Kabat LCDR3

SEQ ID NO:42 sets forth the amino acid sequence of RC-1 IMGT HCDR3

SEQ ID NO:43 sets forth the amino acid sequence of ARN-1 IMGT HCDR3

SEQ ID NO:44 sets forth the amino acid sequence of a Siglec6 peptide

SEQ ID NO:45 sets forth the amino acid sequence of a Siglec6 peptide

SEQ ID NO:46 sets forth the amino acid sequence of hSiglec-6 (V and C2i domains, 27-235)

SEQ ID NO:47 sets forth the amino acid sequence of rhesus macaque Siglec-6 (UniProt accession A0A1D5QH63, 16-226)

SEQ ID NO:48 sets forth the amino acid sequence of human CD33 (21-232)

### DETAILED DESCRIPTION

### I. Overview

Siglec-6 belongs to the family of CD33-related siglecs that share an N-terminal sialic acid-binding domain (V-type Ig-like domain), followed by a variable number of C2-type Ig-like domains, a transmembrane domain, and an intracellular domain that typically bears immunoreceptor tyrosine-based inhibitory motifs (ITIMs). Siglec-6 is a promising target for antibody-based therapy due to its overexpression on CLL cells and coincidental absence on most healthy cell types, with exceptions limited to placental trophoblasts, mast cells and a portion of activated B cells [Siglec-6. The Human Protein Atlas 2021; proteinatlas.org/ENSG00000105492-SIGLEC6; Duan S, Paulson J C. Annu Rev Immunol 2020; 38:365-95; Kardava L, et al. J Clin Invest 2011; 121(7):2614-24].

The present invention is predicated in part on the studies undertaken by the present inventors to probe human antibody libraries for novel and functional Siglec-6 specific human mAbs. The antibody libraries were generated by randomly combining light and heavy chains from either (i) a cancer patient who had been transplanted with a new immune system from a healthy donor or (ii) the bone marrow of 6 healthy donors. The antibody libraries were selected with a recombinant Siglec-6 protein to identify Siglec-6 specific antibodies. A few human mAbs thus identified, e.g., clones RC-1, RC-2, and ARN-1 as detailed herein, selectively bind to Siglec-6.

Initial studies using the anti-Siglec-6 mAb JML-1 in IgG1 format demonstrated a lack of anti-CLL effector functions, which led the inventors to consider alternative mechanisms or antibody formats to translate JML-1 into a potential therapeutic. It is now recognized that CLL patients treated with Bruton's tyrosine kinase inhibitors (BTKis) exhibit increased persistence and activation of T cells, supporting the use of immunotherapies that employ T cells in targeting the latent leukemia that persists during BTKi treatment [Long M, et al. J Clin Invest 2017; 127(8):3052-64; Robinson H R, et al. Blood 2018; 132(5):521-32; Mhibik M, et al. Blood (2021) 138 (19): 1843-1854; Ahn I E, Brown J R. Front Immunol 2021; 12:687458]. Given the success of the clinically approved CD19×CD3 T cell-recruiting bispecific antibody (T-biAb) blinatumomab in acute lymphocytic leukemia and the mechanistic potency of this modality, the inventors further reasoned that T cell engaging bispecific antibodies (T-biAbs) targeting Siglec-6 might be an effective CLL immunotherapy, without the need for transplantation and other high-risk cell therapies [Labrijn A F, et al. Nat Rev Drug Discov 2019; 18(8):585-608; Rader C. Curr Opin Biotechnol 2019; 65:9-16]. Blinatumomab, a CD19×CD3 T-biAb in the ˜50-KDa BITE (tandem single-chain variable fragment (scFv)2 format) is not approved for CLL and it has numerous drawbacks including the elimination of all healthy B cells at all stages of development, the induction of cytokine release syndrome (CRS), and a short circulatory half-life due to its small size. In contrast, an anti-Siglec-6 T-biAb may work well for CLL patients as it will have increased target cell specificity, may result in less systemic cytokine release due to lower target expression, and have a longer circulatory half-life if it is fused to an Fc domain. In the current study the inventors re-probed the post-alloHSCT antibody library and identified additional clones targeting Siglec-6, with higher affinity than the original JML-1 clone. Accordingly, the identified anti-Siglec-6 antibodies were engineered into various T-biAb formats. Some molecules of the invention successfully lyse Siglec-6⁺ cell lines and primary CLL cells. Some molecules of the invention eradicate a Siglec-6⁺ fraction of primary B cells from healthy donors, corroborating the potency and specificity of molecules of the invention. As detailed herein, the dual-affinity re-targeting (DART, a variant of the diabody format) format—which creates a shorter cytolytic synapse between target and effector cell than single-chain variable fragment (scFv)—based T-biAbs—outperformed the scFv at killing target cells and activating T cells. In a mouse model of human CLL, the inventors demonstrated that anti-Siglec-6 DART-Fc inhibits tumor growth and extends survival, comparable to a CD19-targeting control T-biAb. Therefore Siglec-6 appears to be a promising target for T-biAbs, a potent immunotherapy that can successfully eliminate leukemic cells, with less on-target-off-tissue toxicity than current therapies.

Siglec-6 is an emerging target for cancer immunotherapy that offers increased specificity over conventional CLL targets which are universal B cell markers. The inventors identified high-affinity, patient-derived anti-Siglec-6 mAbs that hold translational potential given that they arose in a human allogeneic setting and may have contributed to curing CLL in this patient. The inventors mapped the epitope of these mAbs to the N-terminal lectin domain, raising the possibility there may be functional consequences of interfering with siglec-glycan interactions.

The inventors engineered Siglec-6 clones into T-biAbs and demonstrated the potency of this modality for killing Siglec-6⁺ CLL cells, while sparing the majority of healthy B cells, which are Siglec-G. In vitro, T-biAbs directed the killing of target cells with sub-picomolar potency and activated T cells to secrete type I cytokines. The inventors found that Siglec-6×CD3 scFv-Fc only weakly induced IL-2 secretion, which the inventors attributed to the membrane-distal epitope location on Siglec-6. The inventors were able to improve T-biAb activity via antibody engineering into the DART-Fc format, increasing lysis potency >10-fold and IL-2 levels >2-fold. Mechanistically, neither the binding kinetics nor the number of synapses formed favored the DART-Fc vs. the scFv-Fc, as has been reported when comparing DARTs and BiTEs targeting CD19 [Moore P A, et al. Blood 2011:117(17):4542-51]. Rather, the inventors observed the formation of tighter synapses when using the same anti-Siglec-6 clone in aDART-Fc versus scFv-Fc format, and the reduction in synapse length is known to affect T-biAb function [Bluemel C, et al. Cancer Immunol Immunother 2010; 59(8):1197-209; Santich B H, et al. Sci Transl Med 2020; 12(534):eaax1315]. One limitation of our work was using a flow cytometry-based system that lacks the resolution to measure the distance between opposing membranes in a synapse. As a correlate for synapse length, the inventors measured internuclear distance of floating cell conjugates. The benefit of the approach was the throughput—it allowed the inventors to capture enough events for a Gaussian distribution to reveal that RC-1 clone in the aDART-Fc format held cells closer together than in the scFv-Fc format, but not in comparison to the scFv-Fc targeting CD19, which has one fewer extracellular domain than Siglec-6. Thus, the DART-Fc, which constrains its paratopes by covalent linkage at each end, appears to reduce the effective synapse length by approximately one Ig-like domain. The aDART antibody engineering strategy employed here illustrates a blueprint for targeting membrane-distal epitopes with T-biAbs that can be applied to other targets.

The RC-1/V9 aDART-Fc mediated the killing of CLL cells ex vivo by autologous T cells at endogenous effector-to-target ratio (E:T) for CLL patients (1:10 to 1:100), and lysis correlated with Siglec-6 expression, indicating that Siglec-6 targeted therapies will be most effective for the ˜50% of patients that overexpress Siglec-6. The CD19/V9 scFv-Fc is more efficacious, as one might anticipate given the higher expression of CD19. However, CD19-targeted therapy has not been approved in CLL, and CD19 is a universal B cell marker, like CD20, so its targeting results in humoral immunodeficiency [Herishanu Y, et al. Blood 2021:137(23):3165-73]. The inventors observed that only a fraction of healthy donor B cells are killed ex vivo with RC-1/V9 aDART-Fc, since only ˜23% of healthy B cells express Siglec-6, indicating that Siglec-6 targeting will not eliminate all B cells in vivo, as CD20-targeting mAbs do. Additional studies will be needed to assess the effects on tissue-specific B cell populations and mast cells in vivo. Furthermore, Siglec-6 expression is maintained on CLL cells from BTKi-treated patients, unlike targets such as CD20 which can be downmodulated by BTKi [Pavlasova G, et al. Blood 2016; 128(12):1609-13]. Importantly, treatment of CLL patients with a BTKi has been reported to enhance T cell cytotoxicity in response to a CD19×CD3 T-biAb [Robinson supra. Mhibik M, supra]. Here, the inventors have demonstrated efficacy of Siglec-6×CD3 T-biAbs in peripheral blood mononuclear cells (PBMCs) from patients treated with BTKi. Additional studies on combination treatments with other CLL therapeutics as well as checkpoint inhibitors may further increase responses.

Since little is known regarding the function of Siglec-6 in CLL, the possibility of downmodulation in response to targeted treatment remains a potential concern for therapeutic development that must be investigated further. However, Siglec-6 expression is reportedly higher in the proliferative fraction (CXCR4^{dim}CD5^{bright}) [Calissano C, et al. Molecular medicine (Cambridge. Mass) 2011; 17(11-12):1374-82], so downmodulation may reduce clonal expansion in vivo.

Concurrent to our T-biAb efforts, two groups developed CAR-T cells using our original anti-Siglec-6 clone, JML-1, for the treatment of CLL and AML [Kovalovsky D, et al. Leukemia 2021; 35:2581-91; Jetani H. et al. Blood. 2021 Nov. 11; 138(19):1830-1842]. Their findings corroborate ours and demonstrate a proof-of-concept for targeting Siglec-6-expressing cells with T-cell based therapies. However, our study introduces RC-1, a novel human anti-Siglec-6 clone with 6-fold higher affinity than JML-1. The inventors found that T-biAbs induce higher levels of IL-2 secretion using RC-1 versus JML-1. While the JML-1 CAR-T does not elicit IL-2 in the presence of cells with low target expression [Kovalovsky, supra; Jetani, supra], the RC-1/V9 aDART-Fc results in high levels of IL-2 secretion, even with low target copy number. Additionally, our work expanded the number of CLL patients tested and revealed higher Siglec-6 expression in patients who have received BTKi therapy. In vivo, the RC-1/V9 aDART-Fc eliminated a CLL cell line, extended survival at a low dose of 0.05 mg/kg and demonstrated a half-life of over 1 week. In comparison to CAR-T cells, T-biAbs offer the advantage of being an off-the-shelf therapy for CLL which will be accessible to more patients, at lower cost, and with shorter time-to-treatment.

In conclusion. Siglec-6 is an excellent target for next-generation CLL treatments. Beyond CLL, there are additional indications that exhibit specific expression including AML [Jetani, supra, Nguyen D H, et al., Exp Hematol 2006; 34(6):728-35], mast cell disorders (e.g. mastocytosis) [Duan supra], and prenatal complications (e.g. gestational trophoblastic disease and preeclampsia) [Lam K K, et al. J Biol Chem 2011; 286(43):37118-27; Rumer K K, et al. Endocr Relat Cancer 2012; 19(6):827-40]. Pending further investigation into its immunoinhibitory signaling capacity, solid tumors could also benefit from blocking Siglec-6 on tumor-infiltrating mast cells [Yu Y, et al. Front Immunol 2018; 9:2138].

In accordance with these studies, the present invention provides novel human antibodies and related antigen-binding fragments (antibody fragments) or antibody-based binding proteins thereof that specifically recognize Siglec-6, as well as antibody-drug conjugates (ADCs), T-cell engaging bispecific antibodies (T-biAbs), and chimeric antigen receptor (CAR)-engineered T cells (CAR-Ts) with specific anti-tumor activity in Siglec-6 expressing tumors in vitro and in vivo. Antibodies of the invention are useful as Siglec-6 targeting antibodies. Also provided in the invention are methods of using these antibody agents in therapeutic and diagnostic applications for diseases and conditions associated with Siglec-6 expression, e.g., leukemias, lymphomas, other blood disorders, autoimmune diseases, and inflammatory diseases.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” also synonymously called “immunoglobulins” (Ig), or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993) ), T-cell engaging bispecific antibodies (T-biAbs), NK-cell engaging bispecific antibodies (NK-biAbs), antibodies conjugated to other molecules, such as antibody drug conjugates, radioimmunoconjugates, antibody based binding proteins and chimeric antigen receptors including V_{H} and V_{L} regions.

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA. IgD and IgE, respectively.

Each heavy chain of an antibody is comprised of a heavy chain variable region (vii) and a heavy chain constant region. The heavy chain constant region of most IgG isotypes (subclasses) is comprised of three domains, C_{H}1, C_{H}2 and C_{H}3, some IgG isotypes, like IgM or IgE comprise a fourth constant region domain, C_{H}4 Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (C1q) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

The assignment of amino acids to each V_{L} and V_{H} domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs: the AbM definition used by Oxford Molecular's antibody modelling software; and the contact definition of Martin et al (bioinfo.org.uklabs), and IMGT definition (imgt.org/IMGTScientificChart/Numbering/IMGTnumberingCDR_VK.html) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE 1
Conventional Definitions of CDRs Using Kabat Numbering

| | | | Composite | | | |
|---|---|---|---|---|---|---|
| | | | of Chothia | | | |
| Loop | Kabat | Chothia | & Kabat | AbM | Contact | IMGT |
| L1 | L24--L34 | L24--L34 | L24--L34 | L24--L34 | L30--L36 | L27-L32 |
| L2 | L50--L56 | L50--L56 | L50--L56 | L50--L56 | L46--L55 | L50-L52 |
| L3 | L89--L97 | L89--L97 | L89--L97 | L89--L97 | L89--L96 | L89-L97 |
| H1 | H31--H35B | H26--H32 . . . H34* | H26--H35B* | H26--H35B | H30--H35B | H26-H33 |
| H2 | H50--H65 | H52--H56 | H50--H65 | H50--H58 | H47--H58 | H51-H56 |
| H3 | H95--H102 | H95--H102 | H95--H102 | H95--H102 | H93--H101 | H93-H102 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H3SA is present, it ends at H33. If both H35A and H35B are present, it ends at H34.

An “intact” immunoglobulin is one that comprises an antigen-binding site as well as a CL and at least H chain constant domains. C_{H}1, C_{H}2 and C_{H}3. The constant domains can be native sequence constant domains (e.g., human native sequence constant domains) or amino acid sequence variants thereof. An intact immunoglobulin can have one or more effector functions.

“Immunoglobulin fragments” comprise a portion of an intact immunoglobulin, preferably the antigen binding or variable region of the intact immunoglobulin. Examples of immunoglobulin fragments include, but are not limited to, Fab, Fab′, F(ab′)₂, and Fv fragments: diabodies; linear immunoglobulins (see U.S. Pat. No. 5,641,870, Example 2; Zapata et al., Protein Eng. 8(10): 1057-1062 [1995]); single-chain immunoglobulin molecules; and multispecific immunoglobulins formed from immunoglobulin fragments. In some embodiments, the immunoglobulin fragments include all possible alternate fragment formats. In some embodiments, the immunoglobulin fragments may be bispecific. In some embodiments, the immunoglobulin fragments may be bi-paratopic. In some embodiments, the immunoglobulin fragments may be trispecific. In some embodiments, the immunoglobulin fragments may be multimeric. In some embodiments, an immunoglobulin fragment comprises an antigen binding site of the intact immunoglobulin and thus retains the ability to bind antigen. In some embodiments, the immunoglobulin fragment contains single variable domains which have the ability to bind antigen. In some embodiments, the immunoglobulin fragments are further modified (not limited to peptide addition, pegylation, hesylation, glycosylation) to modulate activity, properties, pharmacokinetic behavior and in vivo efficacy.

Fragments typically compete with the intact antibody from which they were derived from specific binding to their target. Fragments can be synthesized by recombinant techniques or by chemical or enzymatic digestions. Papain digestion of immunoglobulins produces two identical antigen binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an immunoglobulin yields a single large F(ab′)₂ fragment which roughly corresponds to two disulfide linked Fab fragments having bivalent antigen-binding activity and is still capable of cross-linking antigen. Fab fragments differ from Fab fragments by having additional few residues at the carboxy terminus of the C_{H}1 domain including one or more cysteines from the immunoglobulin hinge region. Fab′-SH is the designation herein for Fab′ in which the cysteine residue(s) of the constant domains bear a free thiol group. F(ab′)₂ immunoglobulin fragments originally were produced as pairs of Fab′ fragments which have hinge cysteines between them. Other chemical couplings of immunoglobulin fragments are also known.

The Fc fragment comprises the carboxy-terminal portions of both H chains held together by disulfides. The effector functions of immunoglobulins are determined by sequences in the Fc region, which region is also the part recognized by Fc receptors (FcR) found on certain types of cells.

“Fv” is the minimum immunoglobulin fragment which contains a complete antigen recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the immunoglobulin. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv” are immunoglobulin fragments that comprise the V_{H} and V_{L} immunoglobulin domains connected into a single polypeptide chain. Preferably, the sFv polypeptide further comprises a polypeptide linker between the V_{H} and V_{L} domains which enables the sFv to form the desired structure for antigen binding. For a review of sFv, see Plückthun in The Pharmacology of Monoclonal Antibodies, vol. 113, Rosenburg and Moore eds., Springer-Verlag, New York, pp. 269-315 (1994); and Antibody Engineering, Borrebaeck ed., Oxford University Press (1995).

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) Fe-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments (antigen-binding fragments).

“Binding affinity” is generally expressed in terms of equilibrium association or dissociation constants (K_{A} or K_{D}, respectively), which are in turn reciprocal ratios of dissociation and association rate constants (k_{off} and kₒₙ, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of Siglec-6) are deemed to have the same binding specificity for that entity.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See. e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans el al., Cancer Res. 50:1495, 1990). A test antibody competes with a reference antibody if an excess of a test antibody (e.g., at least 2×, 5×, 10×, 20× or 100×) inhibits binding of the reference antibody by at least 50% as measured in a competitive binding assay. Some test antibodies inhibit binding of the references antibody by at least 75%, 90% or 99%. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “Antibody-Drug Conjugate”, or “ADC” refers to an antibody to which a therapeutically active substance or an active pharmaceutical ingredient (API) has been covalently coupled, such that the therapeutically active substance or an active pharmaceutical ingredient (APT) can be targeted to the binding target of the antibody to exhibit its pharmacologic function. The therapeutically active substance or an active pharmaceutical ingredient can be a cellular toxin that is able to effect killing of the cells targeted by the ADCs, preferably malignant or cancer cells. The covalent attachment of a therapeutically active substance, an active pharmaceutical ingredient or a cellular toxin can be performed in a non-site specific manner using standard chemical linkers that couple payloads to lysine or cysteine residues, or preferably the conjugation is performed in a site-specific manner, that allows full control of conjugation site and drug to antibody ratio (DAR) of the ADC to be generated.

T cell engaging bispecific antibodies (T-biAbs) or bispecific T cell-engagers (BiTEs) are small antibody-based molecules composed of two antigen-binding antibody fragments (scFvs) genetically fused by a flexible peptide linker and capable of redirecting T cells against cancer cells expressing a tumor-associated antigen (TAA). Typically, the two antibody fragments are single-chain variable fragments (scFvs), one binding to the TAA and the other one recognizing CD3 on T cells. A number of T-biAbs, across a variety of formats, have been shown to successfully redirect T cells to kill tumor cells in vivo. See, e.g., Xu et al., Cancer Immunol Res 2015; 3:266-77; Lutterbuese et al., Proc Nat Acad Sci USA 2010; 107:12605-10; Ishiguro et al., Sci Trans Med 2017; 9. pii:eaal4291; Iizuka et al., Clin Cancer Res 2019; 25:2925-34; Wu et al., Mol Cancer Ther 2018; 17:2164-75; Hoseini et al., Blood Ads' 2018:2:1250-8.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, saline, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

A “humanized antibody” is an antibody or antibody fragment, antigen-binding fragment, or antibody-based binding protein comprising antibody V_{H} or V_{L} domains with a homology to human VII or V_{L} antibody framework sequences having a T20 score of greater than 80, as defined by defined by Gao et al. (2013) BMC Biotechnol. 13, pp. 55. A “chimeric antibody” is an antibody or antibody fragment, antigen-binding fragment, or antibody-based binding protein comprising nonhuman (e.g., mouse, rat, rabbit or monkey) V_{H} and V_{L} domains fused to human constant domains.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Natl. Acad. Sci. USA 85:2444, 1988; by computerized implementations of these algorithms (GAP, BFSTFIT, FASTA, and TFASTA in the Wisconsin Genetics Software Package, Genetics Computer Group, Madison, WI): or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

The term “subject” refers to human and non-human animals (especially non human mammals). The term “subject” is used herein, for example, in connection with therapeutic and diagnostic methods, to refer to human or animal subjects. Animal subjects include, but are not limited to, animal models, such as, mammalian models of conditions or disorders associated with elevated Siglec-6 expression such as CLL. ALL, mantle cell lymphoma, neuroblastoma, sarcoma, renal cell carcinoma, breast cancer, lung cancer, colon cancer, head and neck cancer, melanoma, and other cancers. Other specific examples of non human subjects include, e.g., cows, horses, sheep, pigs, cats, dogs, mice, rats, rabbits, guinea pigs, monkeys.

Artificial T cell receptors (also known as chimeric T cell receptors, chimeric immunoreceptors, chimeric antigen receptors (CARS) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells) are genetically engineered T cells armed with chimeric receptors whose extracellular recognition unit is comprised of an antibody-derived recognition domain and whose intracellular region is derived from one or more lymphocyte stimulating moieties. The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28) domain in tandem with a T-cell triggering (e.g. CD3zeta) moiety. By arming effector lymphocytes (such as T cells and natural killer cells) with such chimeric receptors, the engineered cell is re-directed with a pre-defined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polvnucleotide segment may be attached so as to bring about the replication of the attached segment. Vectors capable of directing the expression of genes encoding for one or more polypeptides are referred to as “expression vectors”.

III. Siglec-6 specific antibodies and related derivative molecules

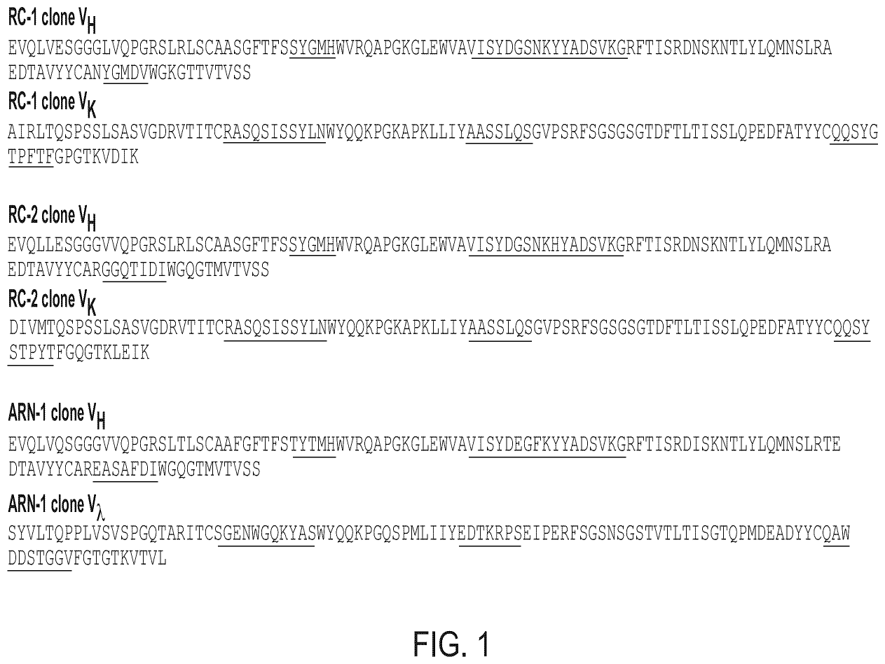

In one aspect, the invention provides antibodies, antigen-binding fragments (aka “antibody fragments”),antibody-based binding proteins ADCs, RICs, T-biAbs, NK-biAbs, or CARS that specifically bind to human Siglec-6 with the same binding specificity as that of anti-Siglec-6 antibodies exemplified herein (FIG. 1). Antibodies of the invention include intact antibodies (e.g., IgG1), antibody fragments or antigen-binding fragments (e.g., scFv or Fab fragments), antibody-based binding proteins, T- or NK-cell engaging bispecific antibodies (T-biAbs or NK-biAbs), radioimmunoconjugates (RICs), ADCs and CARs which contain the antigen-binding portions of an intact antibody that retain capacity to bind the cognate antigen, Siglec-6. Examples of such antibody fragments include (i) a Fab fragment, a monovalent fragment consisting of the V_{L}, V_{H}. C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an intact antibody: (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Antibodies of the invention also encompass antibody fragments (or “antigen-binding fragments”), like single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies of the present invention also encompass single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The various antibodies, antibody-based binding proteins, and antibody fragments thereof described herein can be produced by enzymatic or chemical modification of the intact antibodies, or synthesized de nova using recombinant DNA methodologies, or identified using phage display libraries. Methods for generating these antibodies, antibody-based binding proteins, and antibody fragments thereof are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cal and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)₂ or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, New York, 1998.

In some embodiments, the antibodies, antibody fragments, antibody-based binding proteins, T-biAbs, ADCs or CARs of the invention have heavy chain CDR1. CDR2 and CDR3 sequences and light chain CDR1, CDR2 and CDR3 sequences that are substantially identical to that of the antibodies shown in FIG. 1. The light chain and heavy chain Kabat CDR sequences of the exemplified antibodies are all indicated in FIG. 1. In some of these embodiments, the antibodies, antibody fragments, antibody-based binding proteins, ADCs or CARs have (1) heavy chain CDR1-3 sequences that are substantially identical to SEQ ID NOs:7-9, respectively; and light chain CDR1-3 sequences that are substantially identical to SEQ ID NOs:10-12, respectively; (2) heavy chain CDR1-3 sequences that are substantially identical to SEQ ID NOs: 13-15, respectively; and light chain CDR1-3 sequences that are substantially identical to SEQ ID NOs:16-18, respectively; or (3) heavy chain CDR1-3 sequences that are substantially identical to SEQ ID NOs:19-21, respectively; and light chain CDR1-3 sequences that are substantially identical to SEQ ID NOs:22-24, respectively.

In some embodiments the antibodies, antibody fragments, antibody-based binding proteins, T-biAbs, ADCs or CARS of the invention comprise the heavy chain CDR1-CDR3 and light chain CDR1-CDR3 sequences are identical to the sequences shown in (1) SEQ ID NOs:7-9 and SEQ ID NOs:10-12 (antibody RC-1), (2) SEQ ID NOs:13-15 and SEQ ID NOs: 16-18 (antibody RC-2), or (3) SEQ ID NOs: 19-21 and SEQ ID NOs:22-24 (antibody ARN-1). In other embodiments, the antibodies, antibody fragments, antibody-based binding proteins, T-biAbs. ADCs or CARs of the invention that specifically bind to Siglec-6 contain (a) a light chain variable domain having a sequence that is substantially identical to any one of SEQ ID NOs:2, 4 and 6. (b) a heavy chain variable domain having a sequence that is substantially identical to any one of SEQ ID NOs: 1, 3 and 5, or (c) both a light chain of (a) and a heavy chain of (b). In some embodiments, the antibody comprises both a light chain of (a) and a heavy chain of (b). In some embodiments, the antibodies, antibody fragments, antibody-based binding proteins, T-biAbs, ADCs or CARS of the invention contains (a) a light chain variable domain having at least 90% identity to any one of SEQ ID NOs:2, 4, and 6, (b) a heavy chain variable domain having at least 90% sequence identity to any one of SEQ ID NOs:1, 3, and 5, or (c) both a light chain of (a) and a heavy chain of (b). In some embodiments, the percentage identity can be at least 91%, at least 92%, at least 93%, at least 94%, at least 95%, at least 96%, at least 97%, at least 98%, at least 99%, or even 100%. In some preferred embodiments, the light chain variable domain has at least 95% identity to any one of SEQ ID NOs:2, 4, and 6. In some embodiments, the light chain variable domain has 100% identity to any one of SEQ ID NOs:2, 4, and 6. In some embodiments, the antibody, antibody fragments, antibody-based binding proteins, T-biAbs, ADCs or CARS contains a heavy chain variable domain having at least 90% identity to any one of SEQ ID NOs: 1, 3, and 5. In other embodiments, the percentage identity can be at least 91%, at least 92%, at least 93%, at least 94%, at least 95%, at least 96%, at least 97%, at least 98%, or at least 99%, or even 100%. In some embodiments, the heavy chain variable domain has at least 95% identity to any one of SEQ ID NOs: 1, 3, and 5. In some embodiments, the heavy chain variable domain has 100% identity to any one of SEQ ID NOs:1, 3, and 5.

In some embodiments, the antibodies, antibody fragments, antibody-based binding proteins, T-biAbs, ADCs or CARs of the invention can comprise any heavy chain as described herein (e.g., heavy chains shown in FIG. 1) in combination with any suitable light chain, such as those exemplified herein. Likewise, the antibody can comprise any of the light chains as described above (e.g., light chains shown in FIG. 1) in combination with any suitable heavy chain, such as those exemplified herein. For example, in preferred embodiments, the antibody comprises a light chain having at least 90% identity to SEQ ID NO:2 and a heavy chain having at least 90% identity to SEQ ID NO: 1 (antibody RC-1), a light chain having at least 90% identity to SEQ ID NO:4 and a heavy chain having at least 90% identity to SEQ ID NO:3 (antibody RC-2), or a light chain having at least 90% identity to SEQ ID NO:6 and a heavy chain having at least 90% identity to SEQ ID NO:5 (antibody ARN-1). In some embodiments, the antibody can comprise the light chain and heavy chain sequences respectively shown in (1) SEQ ID NO:2 and SEQ ID NO:1, (2) SEQ ID NO:4 and SEQ ID NO:3, or (3) SEQ ID NO:6 and SEQ ID NO:5. In the various embodiments, percent (%) identity of peptide sequences can be calculated, for example, as 100×[(identical positions)/min(TGA, TGB)], where TGA and TGB are the sum of the number of residues and internal gap positions in peptide sequences A and B in the alignment that minimizes TGA and TGB. See, e.g., Russell et al, J. Mol. Biol., 244: 332-350 (1994).

The antibody of the invention can be any antibody including a full length antibody, an antibody fragment, an antibody-based binding protein that specifically recognizes or binds to the extracellular domain of Siglec-6. For example, the antibody, antibody fragment or antibody-based binding protein can be polyclonal, monoclonal, recombinant, chimeric, humanized or fully human. In some preferred embodiments, the Siglec-6 specific antibodies or related antibody fragments are human, i.e., comprised fully human antibody sequences. Furthermore, the antibody can be of any isotype including without limitation IgA, IgD, IgE, IgG, or IgM. Thus, for example, the antibody can be any IgA such as IgA1 or IgA2, or any IgG such as IgG1, IgG2, IgG3, IgG4, or synthetic IgG. The antibody can also be any antibody fragment or antibody-based binding protein having specificity for the extracellular domain of Siglec-6, such as F(ab)₂, Fv, scFv, IgGACH2. F(ab′)2, scFv2CH3, Fab, VL, VH, scFv4, scFv3, scFv2, dsFv, Fv, scFv-Fc, (scFv)2, a diabody, a bivalent, a bispecific, or a multispecific antibody. The antibody can be any modified or synthetic antibody, including, but not limited to, non-depleting IgG antibodies, T-biAbs. CARS, or other Fc or Fab variants of antibodies.

In addition to a heavy chain as described above, the antibody, antibody-based binding proteins or antibody fragments thereof of the invention can further comprise a light chain selected from a Fab library using sequential naive chain shuffling. Likewise, in addition to a light chain as described above, the antibody of the invention can further comprise a heavy chain selected from a Fab library using sequential naive chain shuffling. In some embodiments, the invention provides antibodies, antibody-based binding proteins or antibody fragments thereof that are conservatively modified variants of the anti-Siglec-6 antibodies exemplified herein. Typically, the variable regions of these variants have an amino acid sequence that is identical to one of these exemplified sequences except for conservative substitutions at one or more amino acid residues. In some embodiments, the antibody, antibody fragments, antibody-based binding proteins, T-biAbs, ADCs or CARs of the invention specifically binds to Siglec-6 and contains at least one CDR having a sequence selected from the group consisting of SEQ ID NOs:7-24. The invention also provides an isolated antibody with specificity for Siglec-6 containing one or more variants of the foregoing CDR sequences or substantially identical CDR sequences. The variant CDR sequences in these antibodies can include 1, 2, or 3 substitutions, insertions, deletions, or combinations thereof in a sequence selected from the group consisting of SEQ ID NOs:7-24. For example, a variant antibody (or fragment thereof) can include one, two, three, four, five, or six of the foregoing CDR sequences.

In some embodiments, however, the variant antibody (or fragment thereof) includes three CDR sequences of the same light or heavy chain, e.g., light chain CDRs shown in SEQ ID NOs:10-12, SEQ ID NOs:16-18, or SEQ ID NOs:22-24; and heavy chain CDRs shown in SEQ ID NOs:7-9, SEQ ID NOs:13-15, or SEQ ID NOs:19-21. In some embodiments, the variant antibody (or fragment thereof) includes six CDR sequences of the same antibody, e.g., (a) SEQ ID NOs:10-12 and SEQ ID NOs:7-9 (antibody RC-1); (b) SEQ ID NOs:16-18 and SEQ ID NOs:13-15 (antibody RC-2); or (c) SEQ ID NOs:22-24 and SEQ ID NOs:19-21 (antibody ARN-1). In some embodiments, the variant antibody contain 6 CDRs that are identical to one of the exemplified antibodies, and on or more amino acid substitutions in the framework regions. In some embodiments, other than identical CDRs, the variant antibody contain a heavy chain variable region and/or a light chain variable region that are respectively at least 900,o, 95% or 99% identical to the heavy chain variable region and/or the light chain variable region of one of the exemplified antibodies.

In some embodiments, the invention provides antibodies, antibody-based binding proteins or antibody fragments thereof with affinity for Siglec-6 of about 10 μM or less, 5 μM or less, 2 μM or less, 1 μM or less, 500 nM or less, 400 nM or less, 300 nM or less, or 200 nM or less. In some embodiments, the antibodies, antibody fragments, antibody-based binding proteins, ADCs or CARS bind to Siglec-6 with an affinity of about 100 nM or less, about 75 nM or less, about 50 nM or less, about 25 nM or less, about 10 nM or less, or about 5 nM or less. In some embodiments, the antibodies, antibody fragments, antibody-based binding proteins, T-biAbs, ADCs or CARS bind to Siglec-6 with an affinity of about 1 nM or less, about 800 pM or less, about 700 pM or less, about 600 pM or less, about 500 pM or less, about 400 pM or less, about 300 pM or less, about 200 pM or less, or about 100 pM or less. Affinity can be measured using art-known techniques, such as ELISA, biolayer inferometry, or surface plasmon resonance.

The antibody, antibody-based binding protein or antibody fragment thereof of the invention can be produced by any suitable technique, for example, using any suitable eukaryotic or non-eukaryotic expression system. In certain embodiments, the antibody is produced using a mammalian expression system. Some specific techniques for generating the antibodies antibody-based binding proteins or antibody fragments thereof of the invention are exemplified herein. In some embodiments, the antibodies, antibody-based binding proteins or antibody fragments thereof of the invention can be produced using a suitable non-eukaryotic expression system such as a bacterial expression system. Bacterial expression systems can be used to produce fragments such as a F(ab)₂, Fv, scFv, IgGACH2, F(ab′)₂, (scFv)₂CH3, Fab, VL, VH, (scFv)₄. (scFv)₃. (scFv)₂, dsFv, Fv, scFv-Fc, and diabodies including DARTs. Techniques for altering DNA coding sequences to produce such fragments are known in the art.

IV. Siglec-6 targeting derivative molecules

The invention also provides Siglec-6 targeting conjugates, fusions and bispecific molecules that are derived from the Siglec-6 antibodies or antibody fragments described herein. These derivative molecules include, e.g., T-biAbs. ADCs or CARS. Typically these derivative molecules are able to specifically bind to Siglec-6 with the same binding specificity as that of an anti-Siglec-6 antibody exemplified herein (e.g., FIG. 1). Some embodiments of the invention are directed to T cell-engaging bispecific antibodies (T-biAbs) in which a Siglec-6 targeting antibody or antigen-binding fragment thereof of the invention is fused or otherwise linked to a T cell-targeting antibody or antigen-binding fragment thereof. Typically, such a bispecific antibody is formed with (1) a V_{H} domain and a V_{L} domain of a T cell targeting antibody, and (2) a V_{H} domain and a V_{L} domain of a Siglec-6 targeting antibody described herein. In some preferred embodiments, the V_{H} domain and the V_{L} domain of the Siglec-6 targeting antibody in the T-biAb contain CDR sequences respectively shown in SEQ ID NOs: 7-12, SEQ ID NOs: 13-18, or SEQ ID NOs: 19-24. In some embodiments, the T cell targeting antibody or antigen-binding fragment is derived from an anti-CD3 antibody. Any CD3 antibodies known in the art may be employed in the practice of the invention. One specific example of anti-CD3 antibodies suitable for the invention is humanized anti-CD3 antibody V9 as exemplified herein (SEQ ID NO:33) (Zhu et al., J. Immunol. 155:1903-10, 1995). In various embodiments, the employed anti-CD3 antibody or antigen binding fragment can contain heavy chain and light chain variable region sequences respectively shown in SEQ ID NO:34 and 35. Another exemplary anti-CD3 antibody suitable for the invention is anti-CD3 (V9 clone) with Fc with aglycosylation N297A and “hole” mutations (Y349C, T366S, L368A, and Y407) (SEQ ID NO:39). Other suitable anti-CD3 antibodies include those as described in, e.g., Bannerji et al., Blood 134:762, 2019; van der Woude et al., Inflammatory Bowel Diseases 16:1708-16, 2020; Herold et al., N. Engl. J. Med. 346:1692-98, 2002; and Bach, Lancet 378:459-60, 2011.

In some T-biAbs of the invention, the V_{H} domain and the V_{L} domain of the T cell targeting antibody forms a first scFv, and the V_{H} domain and the V_{L} domain of the Siglec-6 targeting antibody forms a second scFv, as exemplified herein. Some other T-biAbs of the invention are dual-affinity re-targeting (DART) antibodies. In these molecules, the V_{H} of the first antibody is linked to the V_{L} of the second antibody, while the V_{L} of the first antibody is linked to the V_{H} of the second antibody. In addition. DART antibodies have added a C-terminal inter-chain disulfide bond between the two polypeptides in order to allow the formation and stabilization of heterodimers. See, e.g., Walseng et al., J. Biol. Chem. 291, 9, 19661-73, 2016. Thus, in the DART antibodies of the invention, the V_{H} domain of the T cell targeting antibody and the V_{L} domain of the Siglec-6 targeting antibody forms a first tandem V_{H}-V_{L} fragment via a short peptide linker, and the V_{L} domain of the T cell targeting antibody and the V_{H} domain of the Siglec-6 targeting antibody forms a second tandem V_{H}-V_{L} fragment via a short peptide linker, as exemplified herein.

The DART antibodies of the invention can further have one or both tandem V_{H}-V_{L} fragments further fused to an antibody Fc arm. As exemplified herein, knobs-into-holes mutations are typically employed to facilitate heterodimerization of different Fc domains in the construction of DART antibody-Fc fusions. The use of “knob mutations” and “hole mutations” in Fc fusion dimerization is well known in the art. See, e.g., Merchant et al., Nat. Biotechnol. 16, 677-681, 1998; and Jendeberg et al., J. Immunol. Methods 201, 25-34, 1997.

In some embodiments, the DART antibody-Fc fusions of the invention contain two polypeptide chains each of which has a tandem V_{H}-V_{L} fragment that is fused to a Fc arm, and the two polypeptide chains dimerize via knob and hole mutations respectively introduced into the two Fc arms. Each of the two V_{H}-V_{L} fragments can be independently fused to the Fc arm in either a V_{H}-V_{L}-Fc format or the V_{L}-V_{H}-Fc format. Some of the Fc-containing T-biAbs of the invention are symmetric dimers. In some of these embodiments, the first polypeptide chain contains a tandem V_{H}-V_{L} fragment formed of a T cell targeting V_{L} and a Siglec-6 targeting V_{H} that is fused to the Fc arm containing hole mutations, and the second polypeptide chain contains a tandem V_{H}-V_{L} fragment formed of a Siglec-6 targeting V_{L} and a T cell targeting V_{H} that is fused to the Fc arm containing knob mutations. Examples of such T-biAbs can have (a) a first polypeptide chain containing (i) a Siglec-6 targeting V_{H} domain with CDR sequences respectively shown in SEQ ID NOs:7-9 and (ii) an overall amino acid sequence that is at least 90% identical to SEQ ID NO:26, and (b) a second polypeptide chain containing (i) a Siglec-6 targeting V_{L} domain with CDR sequences respectively shown in SEQ ID NOs:10-12 and (ii) an overall amino acid sequence that is at least 90% identical to SEQ ID NO:27.

In some other symmetric Fc-containing T-biAbs, the first polypeptide chain contains a tandem V_{H}-V_{L} fragment formed of a Siglec-6 targeting V_{L} and a T cell targeting V_{H} that is fused to the Fc arm containing hole mutations, and the second polypeptide chain contains a tandem V_{H}-V_{L} fragment formed of a T cell targeting V_{L} and a Siglec-6 targeting V_{H} that is fused to the Fc arm containing knob mutations. Examples of such T-biAbs can have (a) a first polypeptide chain containing (i) a Siglec-6 targeting V_{L} domain with CDR sequences respectively shown in SEQ ID NOs:10-12 and (ii) an overall amino acid sequence that is at least 90% identical to SEQ ID NO:28, and (b) a second polypeptide chain containing (i) a Siglec-6 targeting V_{H} domain with CDR sequences respectively shown in SEQ ID NOs:7-9 and (ii) an overall amino acid sequence that is at least 90% identical to SEQ ID NO:29.

Some other Fc-containing T-biAbs of the invention are in an asymmetric format that contains 3 chains. In these embodiments, only one of the two tandem V_{H}-V_{L} fragments is fused to a Fc arm. For example, the T-biAbs trimers can have (a) a first polypeptide chain containing a first tandem V_{H}-V_{L} fragment with a C-terminal E-coil motif (or a K-coil motif) that is fused to a first Fc arm, (b) a second polypeptide chain containing the other tandem V_{H}-V_{L} fragment with a C-terminal K-coil motif (or a E-coil motif), and (c) a third polypeptide chain containing a second Fc arm. In these embodiments, one of the Fc arms contains knob mutations, and the other Fc arm contains hole mutations. Also, the first and the second chains dimerize via the K-coil motif and the oppositely charged E-coil motif. In some of these T-biAbs, the first tandem V_{H}-V_{L} fragment can contain a Siglec-6 targeting V_{L} and a T cell targeting V_{H}, and the second tandem V_{H}-V_{L} fragment can contain a T cell targeting V_{L} and a Siglec-6 targeting V_{H}. In some other T-biAbs, the first tandem V_{H}-V_{L} fragment can contain a T cell targeting V_{L} and a Siglec-6 targeting V_{H}, and the second tandem V_{H}-V_{L} fragment can contain a Siglec-6 targeting V_{L} and a T cell targeting V_{H}. As specific exemplification, the T-biAb can have (a) a first polypeptide chain containing (i) a Siglec-6 targeting V_{L} domain with CDR sequences respectively shown in SEQ ID NOs: 10-12 and (ii) an overall amino acid sequence that is at least 90% identical to SEQ ID NO:30, (b) a second polypeptide chain containing (i) a Siglec-6 targeting V_{H} domain with CDR sequences respectively shown in SEQ ID NOs:7-9 and (ii) an overall amino acid sequence that is at least 90% identical to SEQ ID NO:31, and (c) a third polypeptide chain comprises an amino acid sequence that is at least 90% identical to SEQ ID NO:32.

In some derivative molecules of the invention are antibody-drug conjugates (ADCs), which are molecules containing a Siglec-6 targeting antibody, antigen-binding fragment or antibody-based binding protein that is conjugated to a synthetic molecule such as a drug or toxin. The conjugation can be achieved via any type of suitable means. In some embodiments, recombinant engineering and incorporated selenocysteine (e.g., as described in U.S. Pat. No. 8,916,159 issued on Dec. 23, 2014) can be used to conjugate a synthetic molecule. Other methods of conjugation can include covalent coupling to native or engineered lysine side-chain amines or cysteine side-chain thiols. See, e.g., Wu et al., Nat. Biotechnol, 23: 1 137-1 146 (2005). Some embodiments of the invention are directed to dual-variable-domain ADCs (DVD-ADCs). In these compounds, the first variable domain contains a Siglec-6 targeting antibody or antigen-binding fragment disclosed herein. The second variable domain is derived from a catalytic antibody for site-specific payload conjugation. In some of these embodiments, the employed catalytic antibody (e.g., humanized catalytic antibody 38C2 or variant thereof) contains in its combining site a reactive lysine residue or a cysteine residue for attachment of drug moieties. Construction of such ADCs can be readily carried out in accordance with protocols well known in the art. See, e.g., Nanna et al., Nat Commun 8, 1112, 2017; Nanna et al., Methods Mol Biol. 2033:39-52, 2019; WO 2017/049139; and WO 2020/076849. In some embodiments, the ADCs can be obtained by means of site-specific sortase-enzyme mediated antibody conjugation. See, e.g., WO2014/140317; and Dorr et al., PNAS 2014; 111, 13343-8. Many other chemical and enzymatic assembly strategies known in the art can also be employed in the production of the ADCs of the invention. See, e.g., Beck et al., Nat. Rev. Drug Discov. 2017, 16(5):315-337.

The synthetic molecule can be any molecule such as one targeting a tumor. In some embodiments, the synthetic molecule for conjugation to the antibody is a protein (e.g., an antibody) or an RNA or DNA aptamer. In various embodiments, suitable synthetic molecules (“payloads”) for conjugation to the antibody include, e.g., therapeutic agents such as cytotoxic, cytostatic, or antiangiogenic agents, radioisotopes, and liposomes. The radioisotope can be a diagnostic radioisotope or a therapeutic radioisotope. A cytotoxic agent can be a plant, fungal, or bacterial molecule. In some embodiments, the cytotoxic agent for conjugation to the antibody of the invention is a small molecular weight toxin (MW<2,000 Da, preferably MW<1,000 Da), a peptide toxin, or a protein toxin. Many specific examples of these toxins are well known in the art. See, e.g., Dyba et al., Curr. Pharm. Des. 10:2311-34, 2004; Kuyucak et al., Future Med. Chem. 6:1645-58, 2014; Beraud et al., Inflamm. Allergy Drug Targets. 10:322-42, 2011; and Middlebrook et al., Microbiol. Rev. 48:199-221, 1984. In some embodiments, a therapeutic agent is conjugated to the antibody. For example, the therapeutic agent can be a maytansinoid (e.g., maytansinol or DMI maytansinoid), a tubulysin, a taxane, a camptothecin, a duocarmycine, a calicheamicin, a tiancimycin, a cemadotin, a monomethylauristatin (e.g., monomethylauristatin E or monomethylauristatin F), a pyrrolobenzodiazepine (PBD), a PBD dimer, an anthracycline, including a derivative of the highly potent anthracycline PNU-159682, or an alpha-amanitin.

Therapeutic agents suitable for constructing ADCs of the invention also include vincristine and prednisone. In various embodiments, the therapeutic agent that may be employed in the invention can be an antimetabolite (e.g., an antifolate such as methotrexate, a fluoropyrimidine such as 5-fluorouracil, cytosine arabinoside, or an analogue of purine or adenosine); an intercalating agent (for example, an anthracycline such as doxorubicin, nemorubicin, or preferably a derivative of PNU-159682), daunomycin, epirubicin, idarubicin, mitomycin-C, dactinomycin, or mithramycin, or other intercalating agents such as pyrrolobenzodiazepine; a DNA-reactive agent such as calicheamicins, tiancimycins, and other enediynes; a platinum derivative (e.g., cisplatin or carboplatin); an alkylating agent (e.g., nitrogen mustard, melphalan, chlorambucil, busulphan, cyclophosphamide, ifosfamide nitrosoureas or thiotepa); an RNA polymerase inhibitor such as α-amanitin; an antimitotic agent (e.g., a vinca alkaloid such as vincristine, or a taxoid such as paclitaxel or docetaxel); a topoisomerase inhibitor (for example, etoposide, teniposide, amsacrine, topotecan); a cell cycle inhibitor (for example, a flavopyridol); or a microbtubule agent (e.g., an epothilone, a tubulysine, a pre-tubulysine, discodermolide analog, or eleutherobin analog). A therapeutic agent can be a proteasome inhibitor or a topoisomerase inhibitor such as bortezomib, amsacrine, etoposide, etoposide phosphate, teniposide, or doxorubicin. Therapeutic radioisotopes include iodine (¹³¹I), yttrium (⁹⁰Y), lutetium (¹⁷⁷Lu), actinium (²²⁵Ac), praseodymium, astatine (At), rhenium (Re), bismuth (Bi or Bi), and rhodium (Rh). Antiangiogenic agents include linomide, bevacizumab, angiostatin, and razoxane.

In some embodiments, the synthetic molecule can be a label. Labels can be useful in diagnostic applications and can include, for example, contrast agents. A contrast agent can be a radioisotope label such as iodine (¹³¹I or ¹²³I), indium (¹¹¹In), technetium (⁹⁹Tc), phosphorus (³²P), carbon (¹⁴C), tritium (³H), other radioisotope (e.g., a radioactive ion), or a therapeutic radioisotope such as one of the therapeutic radioisotopes listed above. Additionally, contrast agents can include radiopaque materials, magnetic resonance imaging (MRI) agents, ultrasound imaging agents, and any other contrast agents suitable for detection by a device that images an animal body. A synthetic molecule can also be a fluorescent label, a biologically active enzyme label, a luminescent label, or a chromophore label.

In some other embodiments, the synthetic molecule can be a liposome, as described in Bendas, BioDrugs, 15: 215-224, 2001. In such embodiments, the antibody can be conjugated to a colloidal particle, e.g., a liposome, and used for controlled delivery of an agent to diseased cells. In preparing an antibody conjugated to a liposome, e.g., an immunoliposome, an agent such as a chemotherapeutic or other drug can be entrapped in the liposome for delivery to a target cell. In some embodiments, the antibodies, antibody-based binding proteins or antibody fragments thereof of the invention can also have specificity for one or more antigens in addition to Siglec-6. For example, the antibody of the invention can be engineered (e.g., as a bivalent diabody or a conjugated Fab dimer or trimer) to have specificity for Siglec-6 and another tumor antigen, e.g., an antigen associated with neuroblastoma, renal cell carcinoma, breast cancer, gastric cancer, prostate cancer, colon cancer (e.g., colon adenocarcinoma), or breast cancer (e.g., breast adenocarcinoma). The antibody can be engineered to have specificity for Siglec-6 and an antigen that promotes activation or targeting of cytotoxic effector cells.

### V. Polynucleotides, Vectors and Host Cells for Producing Siglec-6 Antibodies

The invention provides substantially purified polynucleotides (DNA or RNA) that are identical or complementary to sequences encoding polypeptides comprising segments or domains of the antibody, antibody-based binding protein or antibody fragment thereof chains described herein. In some embodiments, the polynucleotides of the invention encode the heavy chain or light chain domains sequences shown in FIG. 1. When expressed from appropriate expression vectors, polypeptides encoded by these polynucleotides are capable of exhibiting Siglec-6 antigen binding capacity. Also provided in the invention are polynucleotides which encode at least one CDR region and usually all three CDR regions from the heavy or light chain of the antibodies described herein. Some other polynucleotides encode all or substantially all of the variable region sequence of the heavy chain and/or the light chain of the exemplified antibodies. For example, some of these polynucleotides encode the amino acid sequence of the heavy chain variable region shown in any one SEQ ID NOs: 1, 3, and 5, and/or the amino acid sequence of the light chain variable region shown in any one SEQ ID NOs:2, 4, and 6. Because of the degeneracy of the code, a variety of nucleic acid sequences will encode each of the immunoglobulin amino acid sequences.

The polynucleotides of the invention can encode only the variable region sequences of the exemplified antibodies. They can also encode both a variable region and a constant region of the antibody. Some of polynucleotide sequences of the invention nucleic acids encode a mature heavy chain variable region sequence that is substantially identical (e.g., at least 80%, 90%, 95% or 99°%) to the mature heavy chain variable region sequence shown in any one SEQ ID NOs: 1, 3, and 5. Some other polynucleotide sequences encode a mature light chain variable region sequence that is substantially identical (e.g., at least 80%, 90%, 95% or 99%) to the mature light chain variable region sequence shown in any one SEQ ID NOs:2, 4, and 6. Some of the polynucleotide sequences encode a polypeptide that comprises variable regions of the heavy chain or the light chain of one of the exemplified antibodies. Some other polynucleotides encode two polypeptide segments that respectively are substantially identical to the variable regions of the heavy chain or the light chain of one of the exemplified antibodies.

The polynucleotide sequences can be produced by de novo solid-phase DNA synthesis or by PCR mutagenesis of an existing sequence (e.g., sequences as described in the Examples below) encoding an exemplified functional antibody. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990: Mattila et al., Nucleic Acids Res. 19:967, 1991; and Eckert et al., PCR Methods and Applications 1:17, 1991.

Also provided in the invention are expression vectors and host cells for producing the functional antibodies described herein. Specific examples of plasmid and transposon based vectors for expressing the antibodies are described in the Examples below. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A. B & C (Invitrogen, San Diego, CA), MPSV vectors, and numerous other vectors known in the art for expressing other proteins. Other useful nonviral vectors include vectors that comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a functional antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a functional antibody chain or fragment. These elements typically include an ATG initiation codon and adjacent ribosome binding site (Kozak consensus sequence) or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted functional antibody sequences. More often, the inserted functional antibody sequences are linked to a signal sequences before inclusion in the vector. Vectors to be used to receive sequences encoding the functional antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human, and preferably of human IgG1 antibodies.

The host cells for harboring and expressing the functional antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

The invention further provides non-eukaryotic or eukaryotic cells (e.g., T or NK lymphocytes) that have been recombinantly engineered to produce the antibodies, antibody-based binding proteins or antibody fragments thereof of the invention. The eukaryotic or non-eukaryotic cells can be used as an expression system to produce the antibody of the invention. In some embodiments, the invention provides Siglec-6 targeted immune cells that are engineered to recombinantly express an Siglec-6 specific antibody of the invention. For example, the invention provides a T cell engineered to express an antibody of the invention (e.g., a scFv, (scFv)₂-Fc, or (scFv)₂), which is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular signaling domain, thereby forming a chimeric antigen receptor (CAR) or T-body. In some embodiments, the employed intracellular signaling domain is that derived from T-cell receptor (TCR). TCR intracellular TCR signaling domains that can be used in a CAR (or T-body) include, but are not limited to, CD3ζ, FcR-γ, and Syk-PT signaling domains as well as the CD28, 4-1BB, and CD134 co-signaling domains. Methods for constructing T-cells expressing a CAR (or T-body) are known in the art. See. e.g., Marcu-Malina et al., Expert Opinion on Biological Therapy, Vol. 9, No. 5 (posted online on Apr. 16, 2009).

### VI. Therapeutic and Diagnostic Application

The Siglec-6 antibodies, antigen-binding fragments thereof, antibody-based binding proteins, T-biAbs, ADCs and CARS disclosed herein can be used in various therapeutic and diagnostic applications. While the only healthy cells and tissues that express Siglec-6 are placental trophoblasts, mast cells and a small portion of activated B cells, aberrant Siglec-6 expression is implicated in and associated with the development of several leukemia diseases. These include, e.g., chronic lymphocytic leukemia (CLL) and acute myeloid leukemia (AML). The Siglec-6 specific antibodies and derivative compounds described herein have high therapeutic and diagnostic utility for these diseases. Other than leukemia, several other diseases with aberrant Siglec-6 expression can also be treated with the Siglec-6 antibodies, antibody fragments thereof and other derivative molecules described herein. These include mast cell disorders such as allergy or mastocytosis, pre-eclampsia and gestational trophoblastic disease. In some embodiments, the Siglec-6 targeting antibodies and derivative molecules disclosed herein can be used for treating tumors containing Siglec-6 expressing myeloid-derived suppressor cells (MDSCs). These include, e.g., solid tumor-infiltrating mast cells. As one specific example, expression of Siglec-6 on mast cells infiltrating colorectal carcinomas has been reported. See, e.g., Yu et al., Front Immunol. 9:2138, 2018. Further, the Siglec-6 antibodies could also be useful for blocking suppressive immunoreceptor tyrosine-based inhibitory motif (ITIM) signaling in tumor-infiltrating mast cells or macrophages, reversing the immunosuppressive environment of solid tumors. By targeting these bystander immune cells, the Siglec-6 antibodies or antigen-binding fragments thereof can be employed to treat tumors in general. Siglec-6 antibodies, antibody fragments thereof and other derivative molecules described herein are also useful in treating autoimmune diseases and inflammatory diseases. Siglec-6 antibodies, antibody fragments thereof and other derivative molecules described herein are useful in depleting Siglec-6+ activated B cells in a subject, for example a subject with an autoimmune disease or an inflammatory disease, such as, for example, multiple sclerosis (MS), rheumatoid arthritis (RA), systemic lupus erythematosus (SLE), type 2 diabetes mellitus, cytokine release syndrome, graft-versus-host-disease, HIV-associated immunopathogenesis.

In some embodiments, the invention provides methods for inhibiting cells with aberrant Siglec-6 expression (e.g., CLL or AML cells) by contacting the cells with an antibody, antibody-based binding protein, antibody fragment thereof, T-biAb, ADC or CAR of the invention. The employed antibody compound can be an antibody or antibody fragment that is not conjugated to another molecule. It can also be a derivative compound (e.g., a T-biAb or an ADC) that has the Siglec-6 antibody or antigen-binding fragment thereof (e.g., a scFv or a tandem V_{H}-V_{L} fragment) conjugated to another antibody molecule or a synthetic molecule. The synthetic molecule can be, e.g., a cytotoxic, cytostatic, or antiangiogenic agent, a radioisotope, or a liposome. The method can be used to inhibit Siglec-6 expressing cells in vitro or in a subject (i.e., in vivo). The contacted Siglec-6 expressing cells can be in, for example, a cell culture or animal model of a disorder associated with aberrant expression of Siglec-6. The methods are useful, for example, to measure and/or rank (relative to another antibody) the antibody's inhibitory activity for a specific Siglec-6 cell type. Inhibiting Siglec-6 cells can include blocking or reducing the activity or growth of Siglec-6 cells. Inhibiting can also include the killing of Siglec-6 cells. While the methods are not bound by or limited to any particular mechanism of action, inhibitory activity can be mediated by blocking Siglec-6-mediated signaling or by blocking the signaling of an Siglec-6 associated receptor. Inhibitory activity can also be mediated by recruitment of immune system effectors that attack Siglec-6 cells, e.g., by activating constituents of the antibody-dependent cell-mediated cytotoxicity (ADCC), antibody-dependent cell-mediated phagocytosis (ADCP) or complement systems.

In some related embodiments, the invention provides methods for treating a subject that has, is suspected to have, or is at risk of developing a disorder associated with aberrant expression of Siglec-6 (e.g., CLL or AML). Generally, the methods include administering a therapeutically effective amount of an isolated antibody or antibody fragment thereof. T-biAb. ADC or CAR of the invention to the subject. The antibody can be any anti-Siglec-6 antibody, anti-Siglec-6 antibody fragment, anti-Siglec-6 antibody-based binding protein of the invention as described herein. Thus, the antibody can be chimeric, humanized, synthetic, F(ab)₂, Fv, scFv, IgGACH2, F(ab′)₂, (scFv)₂CH3, Fab, VL, VH, (scFv)₄. (scFv)₃, (scFv)₂, dsFv, or F. In some embodiments, the method includes administering an IgG, an scFv, a dsFv, a F(ab′)₂, a diabody, or a bivalent antibody. The administered antibody, antibody-based binding protein, antibody fragment thereof can be conjugated to a synthetic molecule described above, e.g., a cytotoxic, cytostatic, or antiangiogenic agent, a therapeutic radioisotope, or a liposome. An exemplary cytotoxic agent is Pseudomonas exotoxin A (PE38). Disorders that can be treated include leukemia and other disorders with aberrant Siglec-6 expression, as well as various tumors.

In some embodiments, the invention provides methods for treating a subject that has, is suspected to have, or is at risk of developing a disorder associated with aberrant Siglec-6 expression (e.g., CLL or AML) by adoptive transfer of the genetically engineered T-cells described herein, which express an antibody or antigen-binding fragment of the invention as a CAR that selectively binds Siglec-6. Recombinant technology can be used to introduce CAR-encoding genetic material into any suitable T-cells, e.g., central memory T-cells from the subject to be treated. The T cells carrying the genetic material can be expanded (e.g., in the presence of cytokines). The genetically engineered T-cells are transferred, typically by infusion, to the patient. The transferred T-cells of the invention can then mount an immune response against Siglec-6 expressing cells (e.g., CLL cells) in the subject. The adoptive transfer method can be used, for example, to treat subjects that have or are suspected to have any of the cancers (e.g., CLL or AML) and other disorders associated with Siglec-6. In some embodiments, the foregoing methods of treatment can further include co-administering a second therapeutic agent for treating the disorder associated with aberrant Siglec-6 expression. For example, when the disorder to be treated involves an Siglec-6-expressing cancer, the method can further include co-administration of a cytotoxic, cystostatic, or antiangiogenic or immune-stimulatory agent (e.g. immune-checkpoint inhibitor antibodies, for instance, but not limited to, those binding to PD1, PDL1, CTLA4, OX40, TIM3, GITR, LAG3, BTk, PI3K, Bcl-2, and the like) suitable for treating the cancer. If the cancer is a B-cell malignancy, the method can further include, for example, co-administration of rituximab, alemtuzumab, ofatumumab, ocrelizumab, ibrutinib, acalabrutinib, idelalisib, venetoclax or a CHOP chemotherapeutic regimen.

In some other embodiments, the invention provides a method for detecting in a biological sample an altered level of Siglec-6 (e.g., cell surface Siglec-6), for example, relative to a control, either by FACS, immunohistochemistry (IHC) or Western Blotting. Generally, the method includes contacting a biological sample with an antibody, antibody-based binding protein, antibody fragment thereof of the invention and determining the amount of antibody that selectively binds to material (e.g., cells) in the sample to thereby determine the level of Siglec-6 in the biological sample. A biological sample can be from a cell culture or from a test subject, e.g., a plasma or a tissue sample from a subject that has, is suspected to have, or is at risk of developing a disease or condition associated with elevated Siglec-6 in a subject. A control level desirably corresponds to the Siglec-6 level detected using the same antibody in a corresponding sample(s) from one or more control cultures or disease-free subjects. Methods of using the antibody of the invention to determine Siglec-6 levels can include any immunoassay such as immuno—(Western) blotting, enzyme-linked immunosorbent assay (ELISA), Immunohistochemistry (IHC) and flow cytometry, e.g., fluorescence-activated cell sorting (FACS) analysis.

The methods of detection can be used to screen for the presence of a disorder associated with elevated Siglec-6. The methods include obtaining a sample from a test subject in need of screening, e.g., a subject that has, is suspected to have, or is at risk of developing a disorder associated with elevated Siglec-6. The level of Siglec-6 (e.g., the amount or concentration) in the sample is measured using an antibody, antibody-based binding protein, antibody fragment thereof of the invention, and the level in the sample is compared to a control level of Siglec-6. The control level represents, for example, the mean level (e.g., the amount or concentration) in sample(s) from one or, preferably, multiple control group subjects that do not have a disorder associated with elevated Siglec-6. Alternatively, the control level can correspond to the level or mean level of Siglec-6 in one or more samples taken from the test subject at one or more prior times, such as when the test subject did not have or did not exhibit, a condition associated with elevated Siglec-6. A significantly higher level of Siglec-6 in the biological sample relative to the control level is indicative of a disorder associated with elevated Siglec-6 in the subject.

Additionally, the methods of detection can be used to monitor the progress of a disorder associated with elevated Siglec-6. The method includes obtaining a sample from a subject in need of screening, e.g., a subject having been diagnosed or suspected to have a disorder associated with elevated Siglec-6. The level of Siglec-6 in the sample is measured using an antibody, antibody-based binding protein, antibody fragment thereof of the invention, and the level in the sample is compared to a control level corresponding to the level or mean level of Siglec-6 in one or more samples taken from the test subject at one or more prior times. Levels of Siglec-6 that are significantly elevated or decreased relative to control indicate that the subject's disorder is deteriorating or improving, respectively. The foregoing methods of detection can be used to screen for the presence or to monitor the progress of disorders including, e.g., leukemia (such as CLL and AML) or gestational trophoblastic disease.

### VII. Pharmaceutical Compositions and Combinations

In another aspect, the invention provides pharmaceutical compositions that contain an antibody, an antibody fragment, an antibody-based binding protein, or an ADC as described herein and a pharmaceutically acceptable carrier. Pharmaceutical compositions can be prepared from any of the antibodies described herein. Exemplary compositions include one or more of an antibody having SEQ ID NO:2 (light chain) and/or SEQ ID NO:1 (heavy chain), an antibody having SEQ ID NO:4 (light chain) and/or SEQ ID NO:3 (heavy chain), and an antibody having SEQ ID NO:6 (light chain) and/or SEQ ID NO:5 (heavy chain). Other antibodies, antibody fragments, T-biAbs, or ADCs suitable for the pharmaceutical compositions of the invention include those having a light chain sequence as shown in SEQ ID NOs:2, 4, and 6, and/or a heavy chain sequence as shown in SEQ ID NOs: 1, 3, and 5. Other exemplary compositions of the invention can contain a variant antibody having one, two, three, four, five, or six CDRs selected from the group consisting of SEQ ID NOs:7-24. In some embodiments the antibody includes three CDR sequences of the same exemplified light or heavy chains shown in FIG. 1. These include the heavy chain CDR1, CDR2 and CDR3 sequences and light chain CDR1, CDR2 and CDR3 sequences respectively shown in (1) SEQ ID NOs:7-9 and SEQ ID NOs: 10-12 (antibody RC-1), (2) SEQ ID NOs: 13-15 and SEQ ID NOs: 16-18 (antibody RC-2), or (3) SEQ ID NOs:19-21 and SEQ ID NOs:22-24 (antibody ARN-1). Some exemplary pharmaceutical compositions contain one of the T cell targeting T-biAbs described herein.

In some embodiments, the compositions of the invention contain a carrier for the antibody, the antibody fragment, the T-biAb, or the ADC, desirably a pharmaceutically acceptable carrier. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., a hybrid molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water. Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

Preparation of pharmaceutical compositions of the invention and their various routes of administration can be carried out in accordance with methods well known in the art. See, e.g., Remington: The Science and Practice of Pharmacy. Mack Publishing Co., 20^{th} ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides: hydrogel release systems: sylastic systems; peptide based systems: wax coatings; compressed tablets using conventional binders and excipients: partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The invention also provides kits suitable for carrying out the methods of the invention. Typically, the kits contain two or more components required for performing the therapeutic or diagnostic methods of the invention. Kit components include, but are not limited to, one or more antibodies, antibody fragments thereof, T-biAb or ADC of the invention, appropriate reagents, and/or equipment. In some embodiments, the kits can contain an antibody, antibody-based binding protein, antibody fragment thereof of the invention and an immunoassay buffer suitable for detecting Siglec-6 (e.g. by ELISA, flow cytometry, magnetic sorting, or FACS). The kit may also contain one or more microtiter plates, standards, assay diluents, wash buffers, adhesive plate covers, magnetic beads, magnets, and/or instructions for carrying out a method of the invention using the kit. The kit scan include an antibody, antibody-based binding proteins, antibody fragments thereof of the invention bound to a substrate (e.g., a multi-well plate or a chip), which is suitably packaged and useful to detect Siglec-6. In some embodiments, the kits include an antibody, antibody-based binding proteins, antibody fragments thereof of the invention that is conjugated to a label, such as, a fluorescent label, a biologically active enzyme label, a luminescent label, or a chromophore label. The kits can further include reagents for visualizing the conjugated antibody, antibody-based binding proteins, antibody fragments thereof, e.g., a substrate for the enzyme. In some embodiments, the kits include an antibody, antibody-based binding proteins, antibody fragments thereof of the invention that is conjugated to a contrast agent and, optionally, one or more reagents or pieces of equipment useful for imaging the antibody in a subject.

Generally the antibody or antibody fragment thereof, derivative molecule (e.g., T-biAb or ADC) of the invention in a kit is suitably packaged, e.g., in a vial, pouch, ampoule, and/or any container appropriate for a therapeutic or detection method. Kit components can be provided as concentrates (including lyophilized compositions), which may be further diluted prior to use, or they can be provided at the concentration of use. For use of the antibody of the invention in vivo, single dosages may be provided in sterilized containers having the desired amount and concentration of components.

### Examples

The following examples are provided to further illustrate the invention but not to limit its scope. Other variants of the invention will be readily apparent to one of ordinary skill in the art and are encompassed by the appended claims.

### Example 1. Discovery of New Anti-Siglec-6 Antibodies

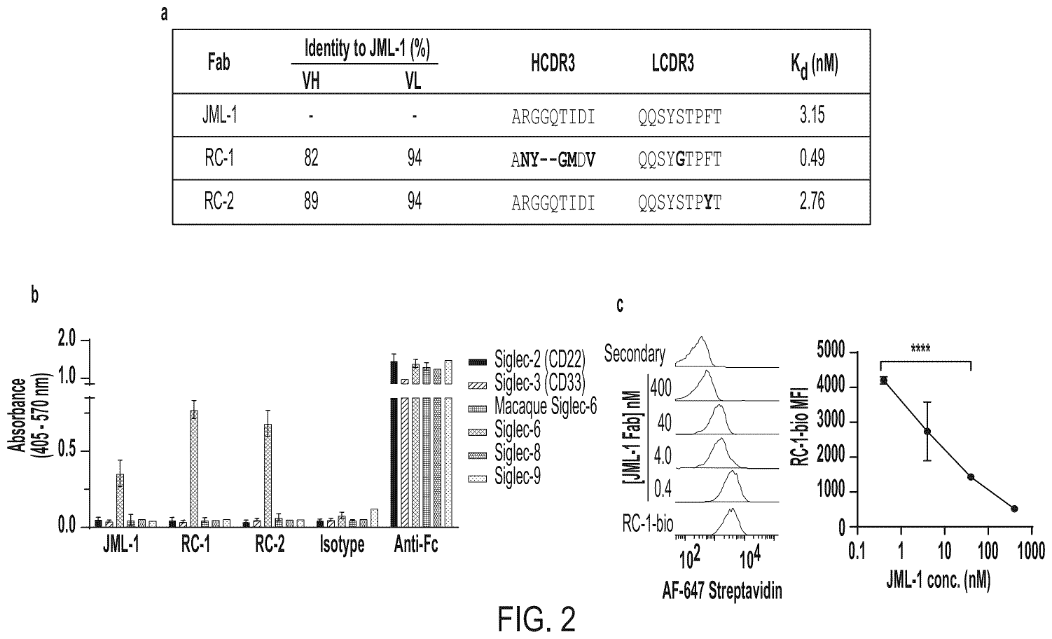

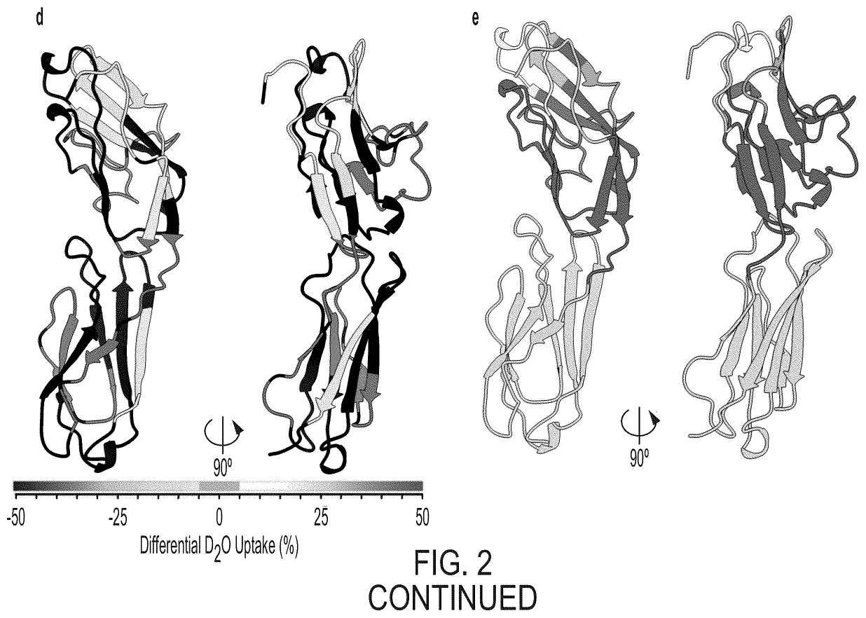

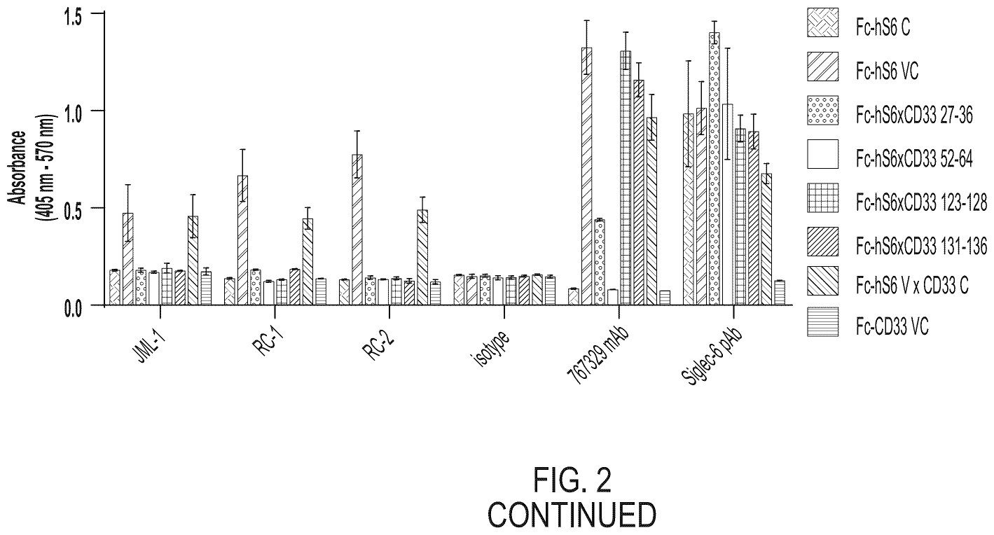

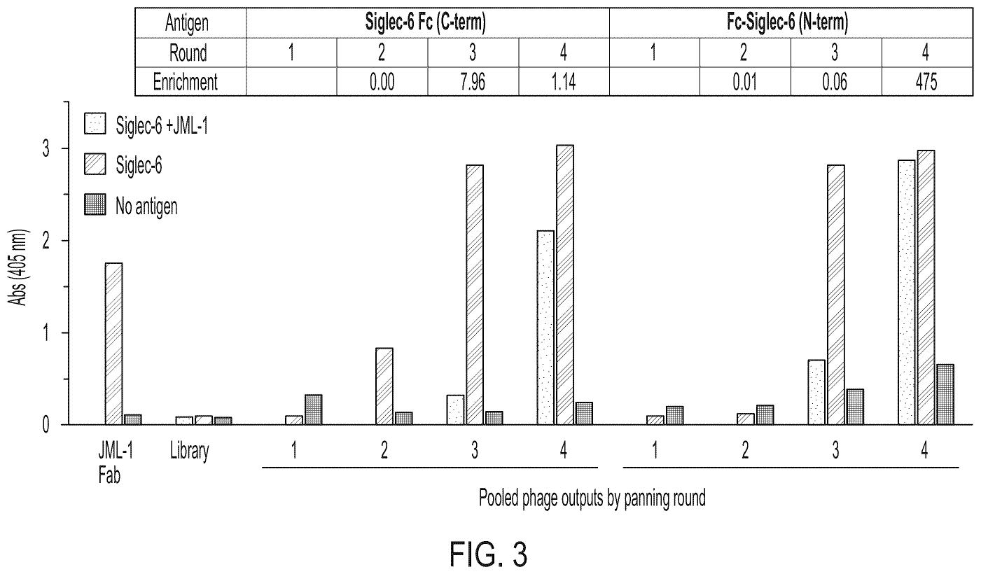

The anti-Siglec-6 mAb JML-1 was discovered in a target-agnostic selection of a post-alloHSCT immune repertoire against primary CLL cells, and the heavy chain complementarity determining region 3 (HCDR3) of JML-1 dominated the phage display selection [Baskar, supra; and Chang, supra]. This raised the question of whether Siglec-6 was broadly targeted in this patient, or if JML-1 was the only anti-Siglec-6 clonotype present in this patient. To identify additional anti-Siglec-6 antibodies in the post-alloHSCT Fab-phage library and validate Siglec-6 as an immunogenic target for the humoral immune response to CLL, the library was re-selected against recombinant human Siglec-6 (hS6) with either C- or N-terminal human IgG1-Fc fusion (hS6-Fc). The Fab-phage panning on immobilized antigen (4 rounds), successfully enriched polyclonal Fabs that bound hS6 in enzyme linked immunosorbent assays (ELISA) (FIG. 3). Sequencing the 4^{th} round outputs revealed most clones had an identical HCDR3 compared to JML-1. The only exception was RC-1, which deviated from JML-1 in both V and J gene usage (FIG. 2a). HCDR1 and HCDR2 were also different for RC-1, and the RC-2 clone shared these new features while maintaining the JML-1 HCDR3. RC-2, had the HV3-30*03 heavy chain variable region (V_{H}) germline of RC-1 but a novel KJ2*01 kappa light chain joining region germline. To determine the binding kinetics of the selected Fabs, surface plasmon resonance (SPR) analysis was employed using immobilized hS6-Fc and soluble Fab. A K_{d} of 0.49 nM was determined for RC-1, approximately 6-fold lower than JML-1's K_{d} of 3.15 nM. The RC-2 clone exhibited a K_{d} of 2.76 nM (FIG. 2a, 3, 4). RC-1 and RC-2 maintained specificity for hS6 and did not bind related human siglec family proteins as determined by ELISA (FIG. 2b). Notably, neither bound the closely related rhesus macaque (Macaca mulatta) Siglec-6 (85% amino acid sequence identity within the ectodomain).

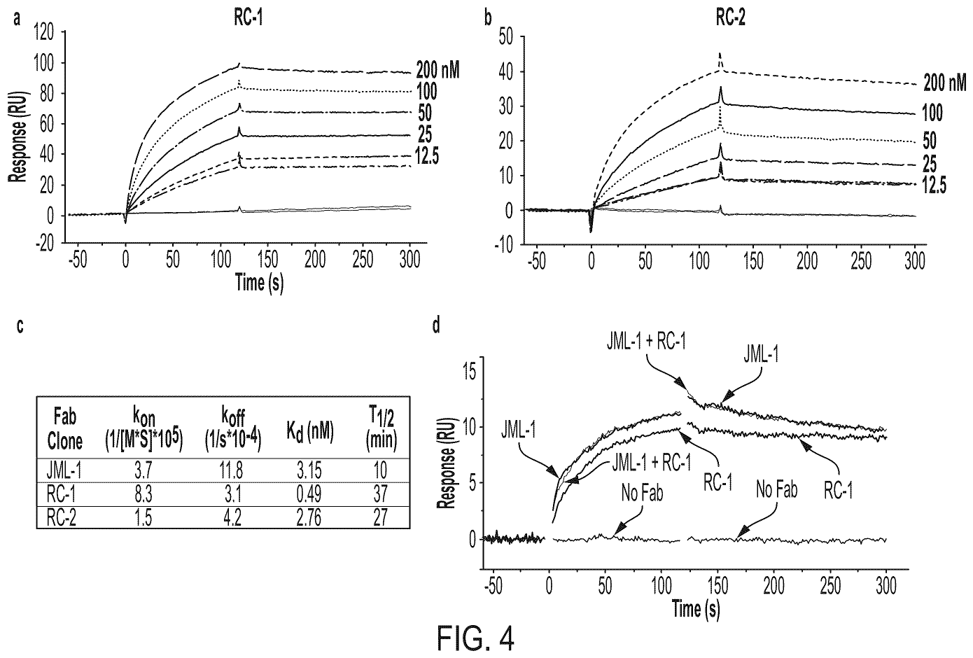

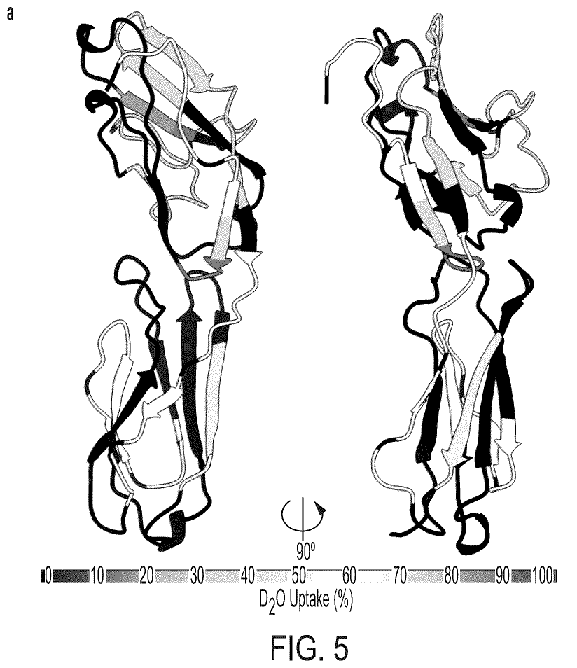

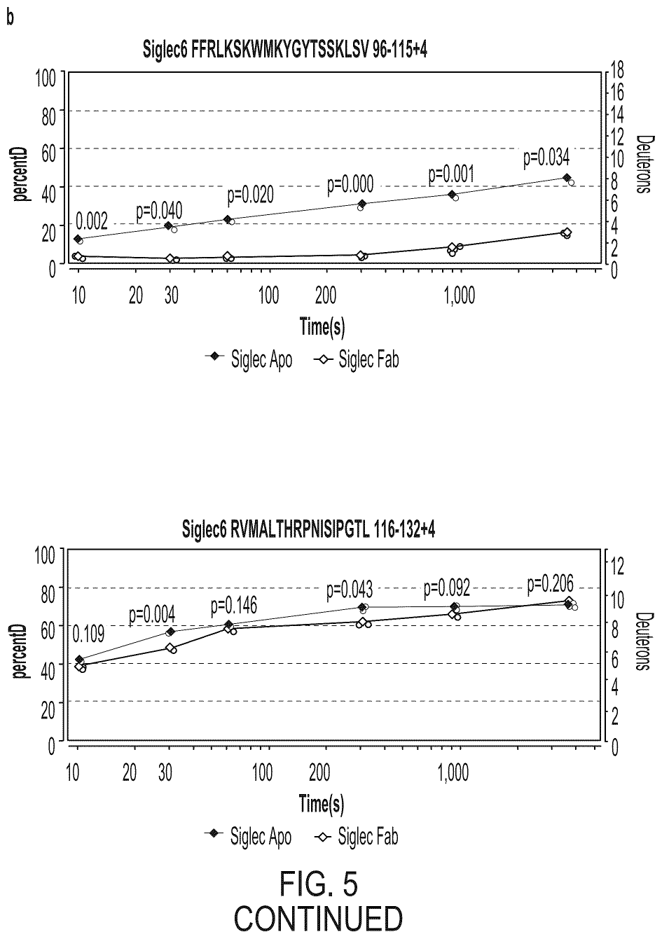

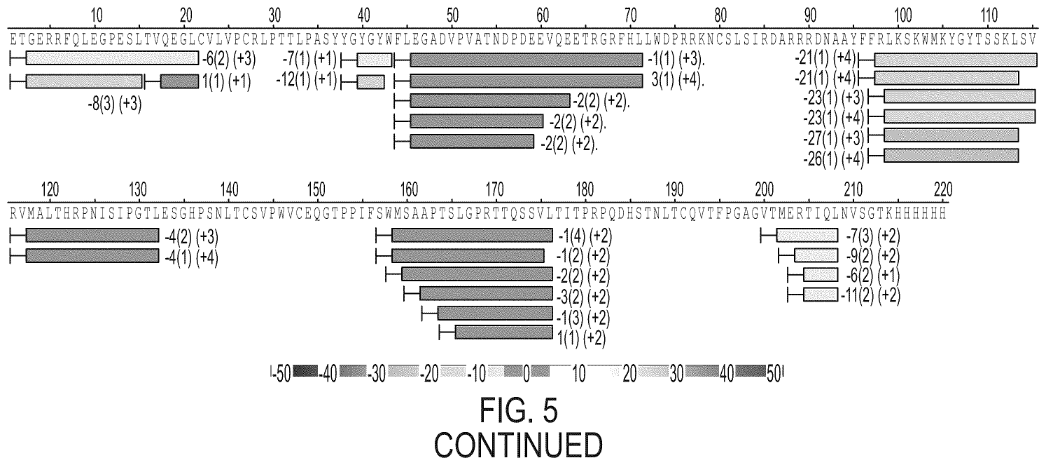

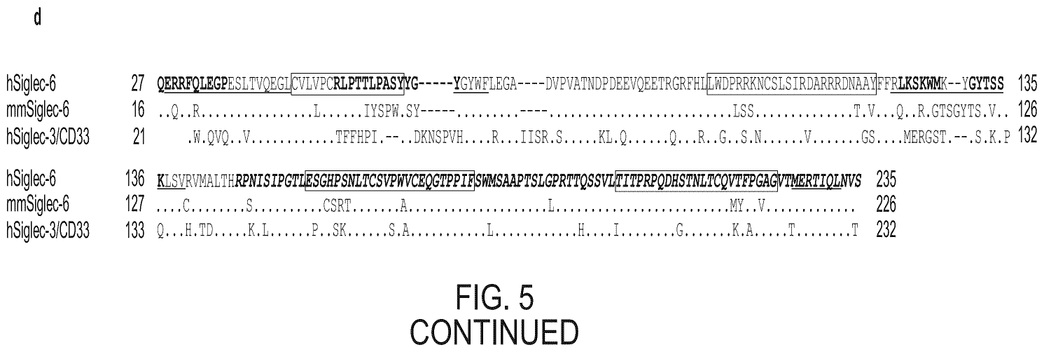

Given the unique HCDR3 and high affinity of RC-1, along with the evidence that polyclonal phage pools bound hS6 in the presence of JML-1 Fab (FIG. 3),the inventors hypothesized RC-1 may bind a distinct epitope from JML-1. To this end, U937 (hS6⁺) cells were blocked with a titration of JML-1 Fab and subsequently stained with biotinylated RC-1 Fab (RC-1-bio. In the absence of JML-1, RC-1-bio bound strongly. However when blocking with equimolar or excess JML-1, RC-1-bio binding was abolished, suggesting that the two clones bind identical or overlapping epitopes on hS6 (FIG. 2c). SPR corroborated this result as the combination of RC-1 and JML-1 did not increase binding in comparison to JML-1 alone (FIG. 4d). To map the epitope targeted by these clones, the hS6 V-type (V) and C2-type I C2-type I (C2i) Ig-like domains, previously shown to be required for JML-1 binding [Kovalovsky, supra], were expressed as tandem-domain and analyzed by HDX-MS (FIG. 5a, Table 2) [Marciano D P, et al. Curr Opin Struct Biol 2014, 28:105-11]. Deuterium (²H) uptake in the presence or absence of RC-1 Fab was compared to identify residues involved in the epitope (FIG. 2d, 5b, 5c), yet one third of the residues were not observed in the MS data (FIGS. 2d, 5b, 5c). To assess the possibility of these missing residues forming the epitope, the sequence of hS6 was aligned with human CD33 (Siglec-3), and rhesus Siglec-6, which both are highly homologous to hS6 but are not bound by the Fabs. Chimeric hS6: CD33 mutants were rationally designed by individually replacing peptides that were implicated by or absent from the HDX-MS data and unique to hS6 (FIG. 2e, 5d). In an ELISA, none of these 4 peptide replacement mutants were bound by the human Fabs, though 3 were bound by a commercial mouse mAb (FIG. 2f). Only the chimera that retained the hS6 V domain, but replaced the C2i domain was bound by JML-1, RC-1, and RC-2. Taken together, these data suggest that the human Fabs bind to a conformational epitope in the membrane distal portion of the hS6 V domain.

TABLE 2
HDX-MS experimental conditions and data analysis parameters
from the guidelines of the IC-HDX-MS community.

| Data Set (FIGS. 2 and 5) | Siglec-6 +/− RC-1 Fab |
|---|---|
| HDX reaction details | 10 mM Sodium phosphate, 150 mM |
| | NaCl, pH = 7.4, 4° C. |
| HDX quench reaction details | 3M Urea, 1% TFA, pH = 2.5, 4° C. |
| HDX time course (seconds) | 10, 30, 60, 300, 900, and 3600 |
| Back-exchange | estimated from input recovery |
| | estimate of 0.7 and deuterium |
| | solution concentration of 0.8 |
| # of Peptides | 28 |
| Sequence coverage | 55% |
| Average peptide length/ | 10/3.653 |
| Redundancy | |
| Replicates (biological or | 3 technical replicates |
| technical) | |
| Repeatability | 3.120461 (average STD) |
| Significant differences in HDX | >5% D (unpaired t-tests at each |
| | time point, p-value <0.01) |

HDX = hydrogen deuterium exchange, TFA = trifluoroacetic acid, % D = percent deuterium

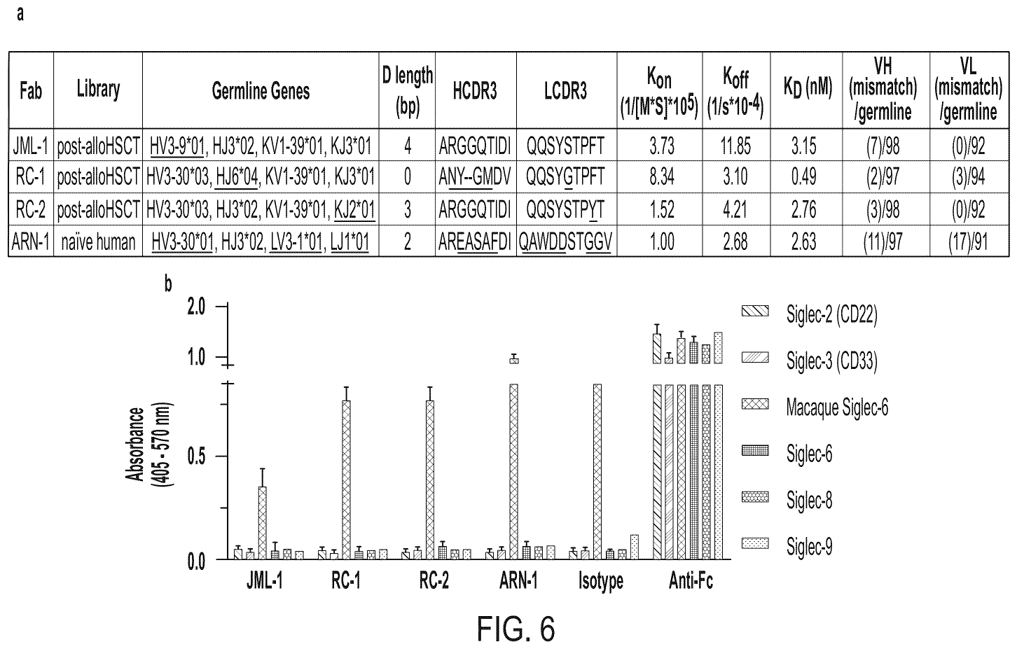

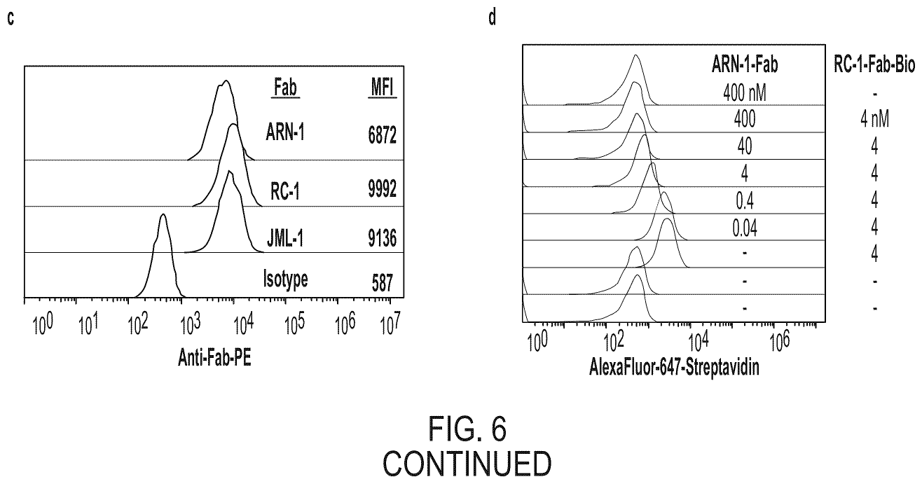

In an effort to identify Fabs that target alternative epitopes on Siglec-6, a naïve human Fab library (Kwong et al., J. Mol. Biol. 384, 1143-1156, 2008) was selected against Fc-hS6. After 4 rounds of panning, a single Fab clone, ARN-1, emerged that had specificity for Siglec-6. ARN-1 exhibits unique IGHV, IGLV, and IGLJ germline gene usage, resulting in entirely new HCDR3 and LCDR3 sequences, yet its affinity for Siglec-6 is similar to the JML-1 family and it binds competitively with RC-1 (FIG. 6a-d).

### Example 2. Targeting Siglec-6 with T-biAbs

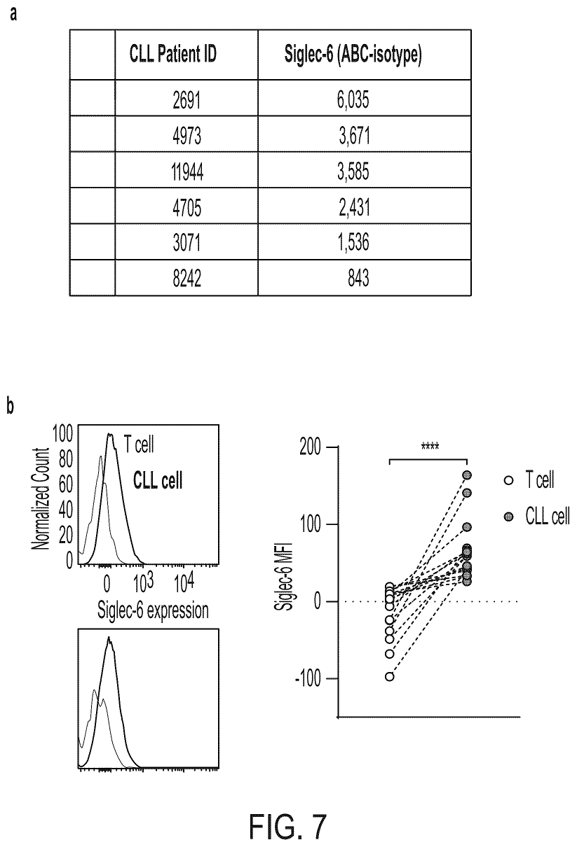

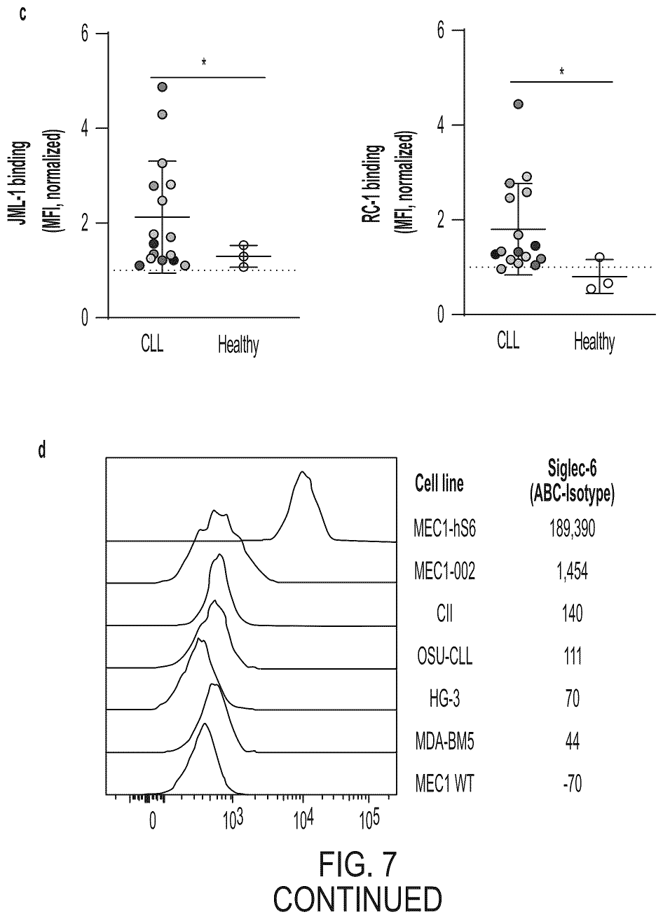

In previous experiments. JML-1 in IgG1 format failed to induce apoptosis or mediate antibody dependent cellular cytotoxicity (ADCC) and complement dependent cytotoxicity (CDC) of primary CLL cells ex vivo, likely due to the low copy number of Siglec-6 on primary CLL cells which varied from 843 to 6,035 (n=6) on the initial treatment naïve cohort the inventors assessed (FIG. 7a, Table 3)

TABLE 3
CLL patient characteristics

| | | | | | | IGHV | | |
|---|---|---|---|---|---|---|---|---|
| | | | | | | Status | | ALC |
| Patient | Siglec-6 | | Age/ | Rai | Treatment | (mutated/ | | (×10³/ |
| ID | MFI | E:T | Gender | Stage | History | unmutated) | Cytogenetics | μL) |
| 2691 | 161.0 | 1:15.2 | 58/M | 2 | TN | U | N/A | 113.52 |
| 3071 | 147.0 | 1:14.5 | 67/M | 4 | TN | M | trisomy 12 | 131.32 |
| 11944 | 96.8 | 1:47.6 | 53/M | 2 | TN | U | trisomy 12 | 278.35 |
| 4086 | 69.4 | 1:35.7 | 53/M | 2 | TN | U | N/A | 94.86 |
| 4973 | 66.6 | 1:27.0 | 50/M | 4 | TN | M | del13q, | 68.97 |
| | | | | | | | del17p | |
| 8242 | 64.3 | 1:58.8 | 81/F | 1 | TN | M | del13q | 202.06 |
| 4705 | 61.4 | 1:33.3 | 58/M | 1 | TN | M | N/A | 49.5 |
| 4653 | 59.6 | 1:71.4 | 46/M | 1 | TN | M | del13q | 153.59 |
| 2356 | 59.4 | 1:8.7 | 31/M | 4 | TN | M | del13q, | 4.7 |
| | | | | | | | del11q | |
| 4296 | 42.8 | 1:27.8 | 74/F | 2 | RR | N/A | N/A | 142.3 |
| 2501 | 42.4 | 1:200.0 | N/A/F | N/A | TN | N/A | N/A | N/A |
| 10515 | 41.5 | 1:37.0 | 66/F | 1 | TN | M | del13q | 123.11 |
| 5403 | 38.9 | 1:500.0 | 65/F | 3 | RR | N/A | del13q, | 238.86 |
| | | | | | | | del17p | |
| 11682 | 33.7 | 1:15.6 | 55/F | 1 | TN | U | Normal | 150.38 |
| 5022 | 32.0 | 1:100.0 | 48/M | 3 | TN | U | del13q, | 557.64 |
| | | | | | | | del17p | |
| 5918 | 25.8 | 1:27.8 | 65/F | 1 | TN | M | del13q | 53.45 |

Available information for the n = 16 CLL patients initially assessed for Siglec-6-targeting therapy. MFI = mean fluorescence intensity; E:T = effector to target ratio; TN = treatment naive; RR = relapsed/refractory; IGHV = immunoglobulin heavy chain status: M = mutated, U = unmutated; ALC = absolute lymphocyte count; N/A = not available.

The inventors turned their attention to T cell activating therapies because they have the potency to eliminate cells with very low target copy number [Hsiue E H, et al. Science 2021; 371(6533):eabc8697], and T-biAbs have shown promise in CLL for other targets [Robinson supra; de Weerdt I, et al. Clin Cancer Res 2021; 27(6):1744-551. Furthermore, both Kovalevsky et al. (Kovalovsky, supra) and Jetani, et al. (Jetani, supra]. recently demonstrated the absence of Siglec-6 on healthy tissues including HSCs and most healthy B cells, indicating Siglec-6 targeting T-biAbs would have less risk of on-target-off-tissue toxicity and may help reverse immune dysfunction in CLL patients' immune dysfunction [Kovalovsky, supra; Jetani, supra]. However, it has been recently reported that Siglec-6 is expressed on T cells in the peripheral blood of bladder cancer patients [Benmerzoug S, et al. Eur Urol Focus. 2022 May; 8(3):748-751]. Since little is known regarding Siglec-6 expression on T cells in the context of CLL. Siglec-6 expression was assessed on the CD3 population of CLL-derived PBMC (FIG. 7b). No specific binding of anti-Siglec-6 mAbs to T cells was detectable when compared to isotype controls, confirming that the target is specific to the CLL B cells.

To validate that the two lead anti-Siglec-6 post-alloHSCT mAbs bind to CLL cells from this patient cohort, JML-1 and RC-1 were used to stain whole PBMC from CLL patients or healthy donors (FIG. 7c). Both JML-1 and RC-1 stained CLL-derived PBMC more than the healthy donor cells. Despite the Siglec-6 expression observed on primary CLL cells, the only Siglec-6 CLL cell line that the inventors have found is MEC1-002-a MEC1 subline that was sorted for JML-1 binding and expanded [Chang, supra] (expression quantified in FIG. 7d). However, since MEC1-002 cells overexpress both FCRL4 and Siglec-6, relative to the parental MEC1 cells [Chang, supra], both of which functionally inhibit B-cell receptor signaling [Kardava L. et al., J Clin Invest 2011; 121(7):2614-24] resulting in slower growth in vitro and in vivo [Kovalovsky, supra], the inventors transduced MEC1 to stably express Siglec-6 (without FCRL4) and fLuc to generate the new clonal cell line, MEC1-hS6 (FIG. 7d) which the inventors employed in proof-of-concept experiments.

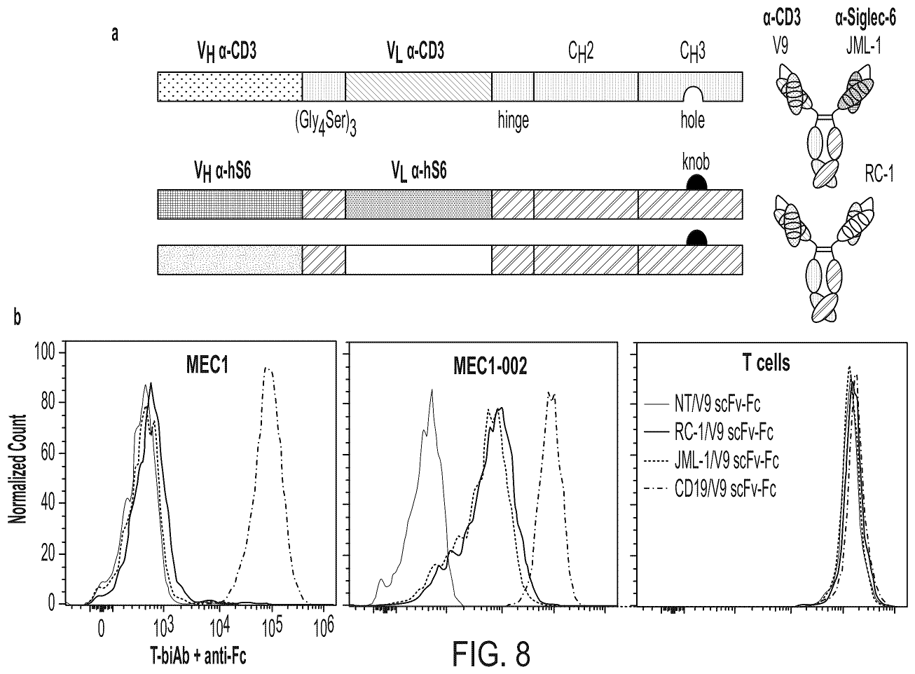

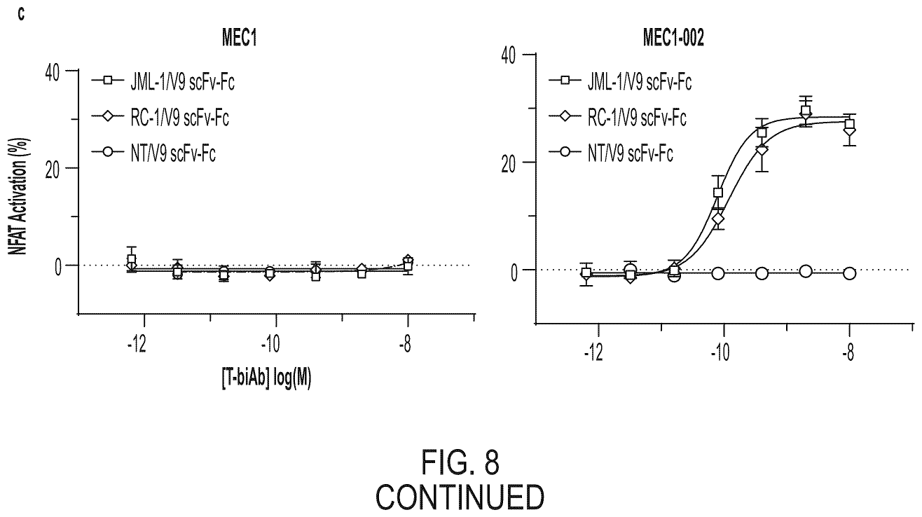

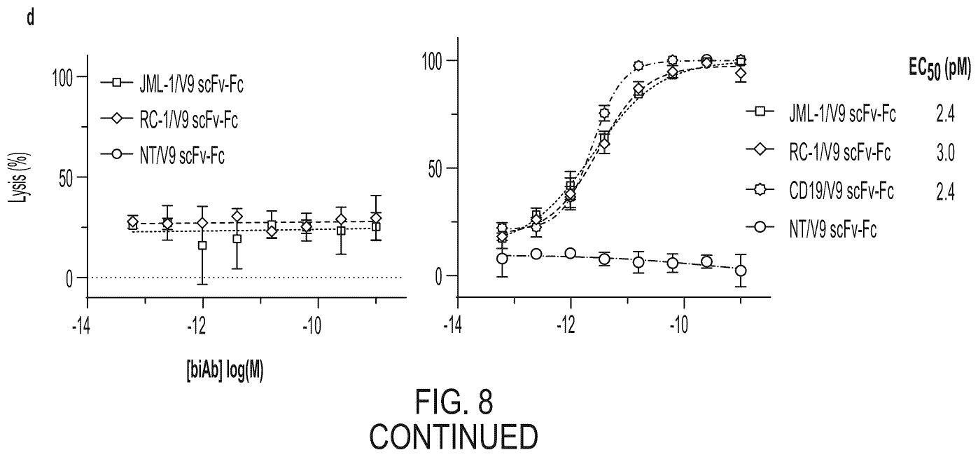

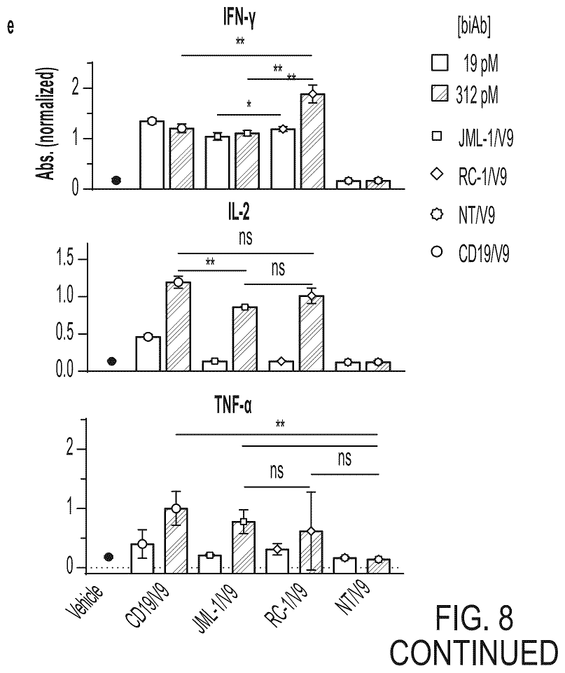

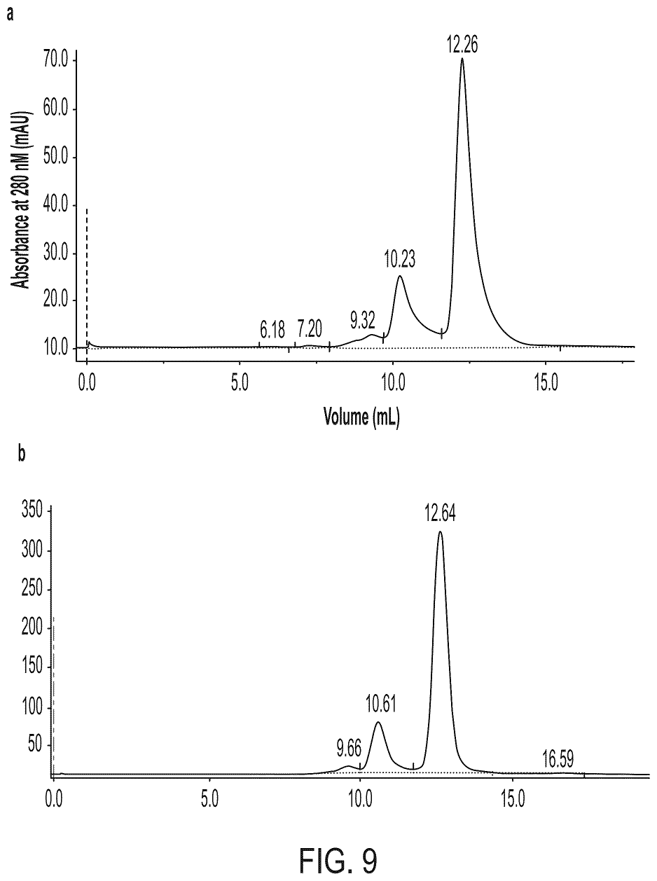

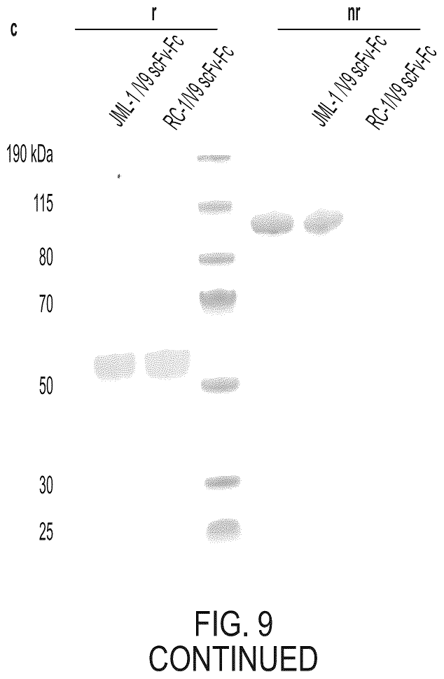

Siglec-6×CD3 T-biAbs were first generated in the IgG-like scFv-Fc format (FIG. 8A). The anti-CD3T-cell engaging scFv arm (V9) of these T-biAbs was paired to Siglec-6-targeting or control (non-targeting, NT) scFv-Fc using knobs-into-holes mutations [Merchant et al., Nat. Biotechnol. 16, 677-681, 1998]. JML-1/V9 scFv-Fc and RC-1/V9 scFv-Fc generated and validated for specificity against Siglec-6⁻, Siglec-6⁺, and CD3⁺ cells (FIG. 8b, 9a-c). To determine whether these T-biAbs elicit T cell signaling in the presence of Siglec-6-expressing cells, a Jurkat T cell line with an nuclear factor of activated T cells (NFAT)-dependent luciferase was co-cultured with T-biAbs and MEC1-002 (fLuc⁻) cells at ratio of 1:1. When the anti-Siglec-6 T-biAbs were added in the presence of MEC1-002 cells, NFAT activation occurred (FIG. 8c). Both RC-1 and JML-1 clones potently activated NFAT signaling, whereas an isotype NT control scFv-Fc did not.

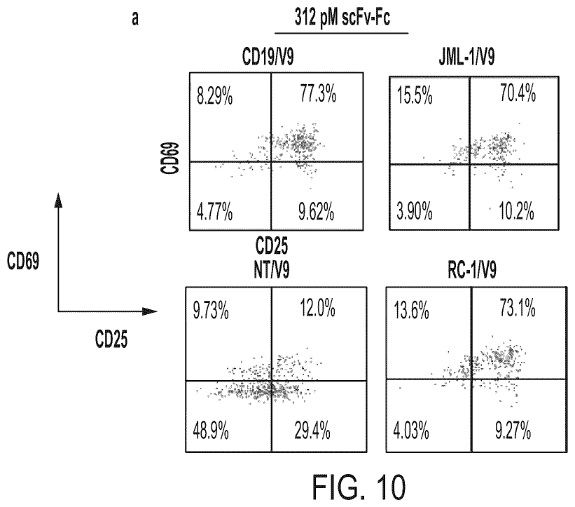

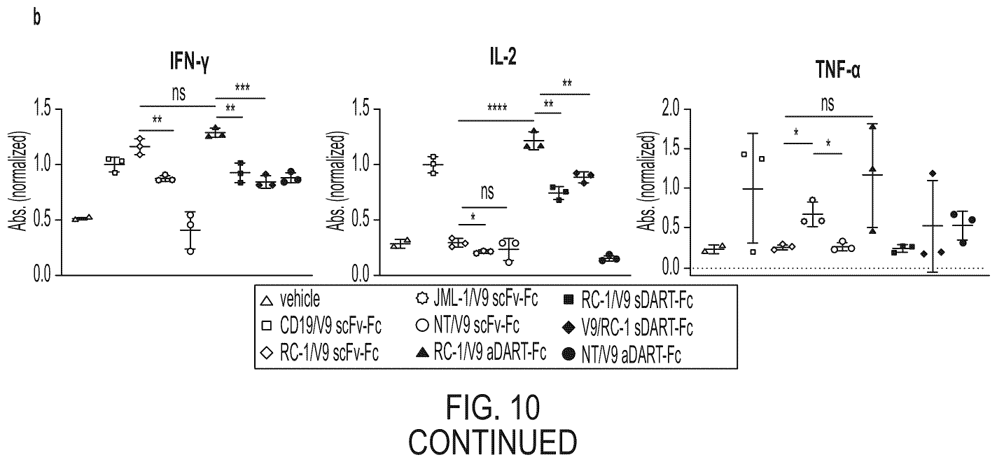

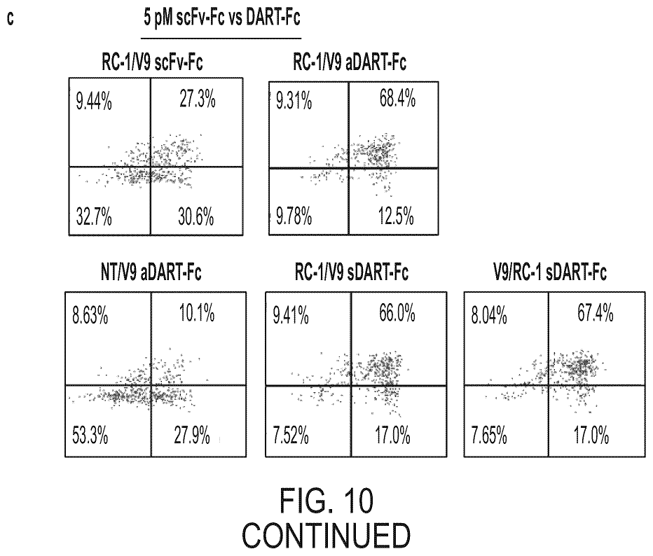

To assess their cytolytic activity T-biAbs were added to co-cultures of healthy donor T cells and fLuc-expressing target cells. Specific lysis of MEC1-hS6 was comparable for CD19/V9 (half maximal effective concentration (EC₅₀)=2.4 pM), JML-1/V9 (EC₅₀=2.4 pM), and RC-1/V9 (EC₅₀=3.0 pM) scFv-Fc, and T cells upregulated CD69 and CD25, key markers of T cell activation\ (FIGS. 8d, 10a). Importantly, there was no appreciable lysis of the Siglec-6 MEC1 cells. In these cytotoxicity experiments, both Siglec-6 targeting T-biAbs induced IFN-γ secretion, but it was significantly higher for RC-1/V9 scFv-Fc compared to JML-11V9 scFv-Fc or CD19/V9 scFv-Fc (FIG. 8e), IL-2 levels were similar between the RC-1 clone and the anti-CD19 control, yet the JML-1 clone induced lower levels of IL-2 secretion. TNF-α secretion was higher for JML-1 and CD19 than for the NT control, while RC-1 was not statistically different from the JML-1 or the NT control (FIG. 8e). Analogous cytokine profiles were induced in the presence of MEC1-002 cells, which express lower levels of Siglec-6 (FIG. 10b). These data suggest that the higher Siglec-6 affinity of RC-1 may result in stronger activation of T cells (higher IFN-γ and IL-2 secretion) than the JML-1 clone. However, the relatively low IL-2 secretion in the presence of MEC1-002 cells indicated that the scFv-Fc T-biAbs may not succeed in fully activating and expanding T cells in vivo, so alternative T-biAb formats were considered.

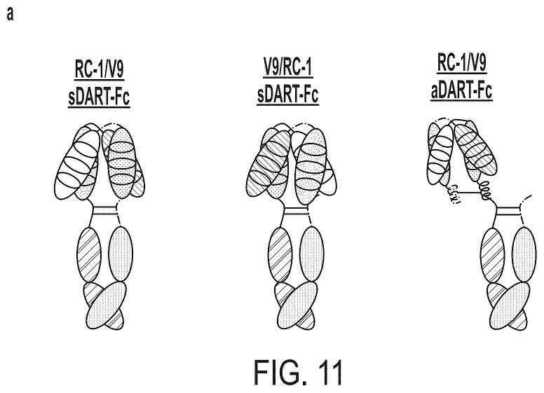

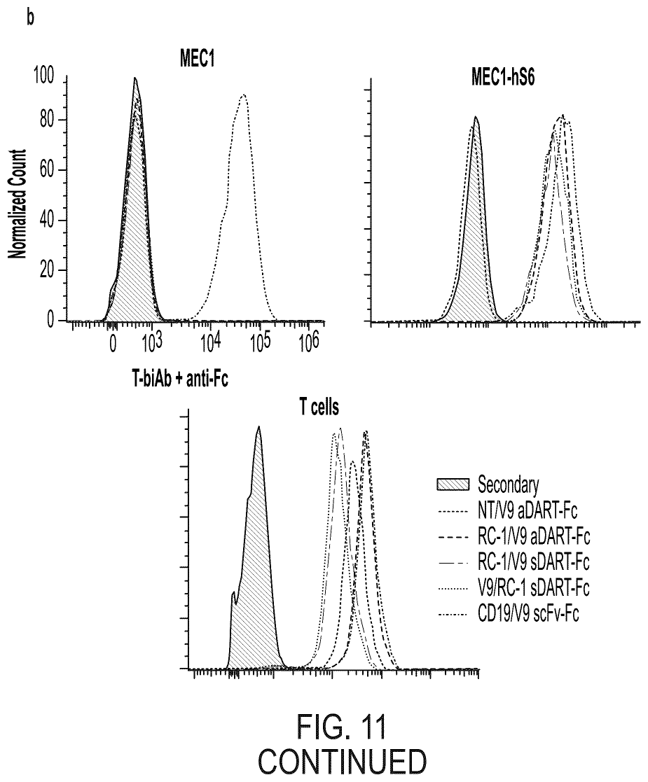

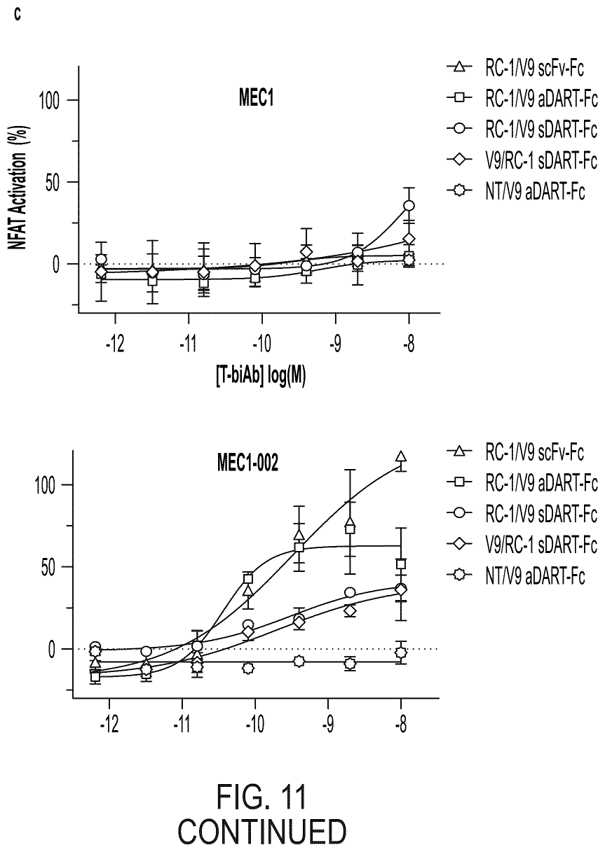

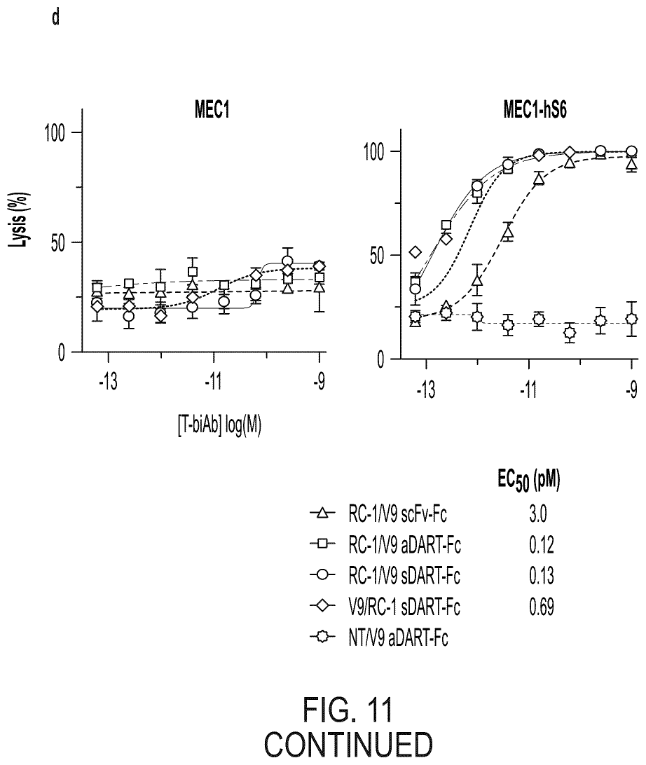

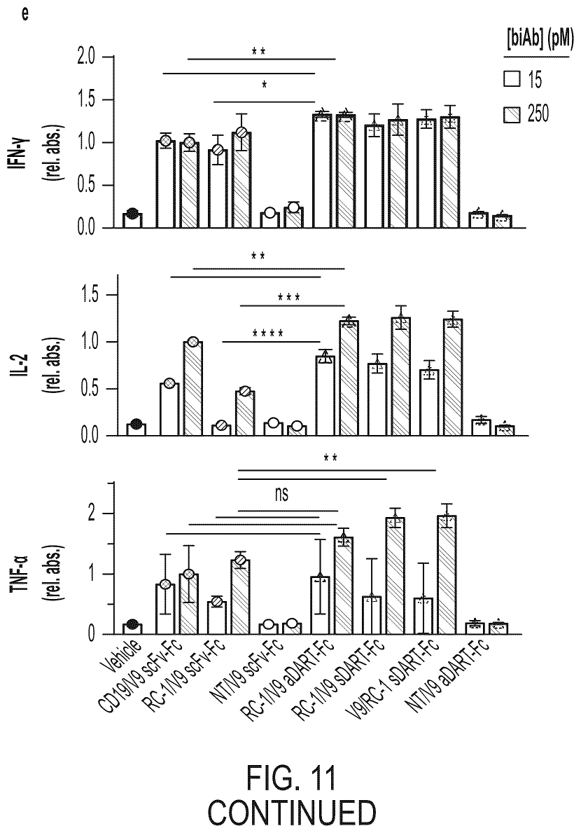

It is well-established that T-biAbs that bind to membrane-proximal epitopes on target cells have greater efficacy than those that bind to membrane-distal epitopes. The theory is that shorter cytolytic synapses—those closer in size to the endogenous TCR-MHC complex-result in the exclusion of large immuno-inhibitory phosphatases from the cytolytic synapse, reducing TCR signaling and cell lysis potential [Qi J, et W. Proc Natl Acad Sci USA 2018:115(24): E5467-E5476: Bluemel C, et al. Cancer Immunol Immunother 2010; 59(8):1197-209]. Since the epitopes of all 3 anti-Siglec-6 mAbs from the post-alloHSCT library are in the outermost V domain (the 3^{rd} Ig-like domain from the surface), the inventors were faced with a significant challenge. The inventors hypothesized the synapse length could be shortened by using the compact and rigid diabody format known as DART [Johnson S. et al., J Mol Biol 2010,399(3):436-49]. For our DART-Fc engineering studies, the inventors chose to pursue RC-1 since it has higher affinity for Siglec-6 and elicited higher IL-2 and IFN-γ secretion than JML-1 in the scFv-Fc format. Two DART formats were employed: a symmetric (sDART) architecture to form a dimer and another with an asymmetric (aDART) architecture to form a trimer (FIG. 11a) [Root A R, et al. Antibodies (Basel) 2016 Mar. 4; 5(1):6; Moore P A, et al. Blood 2011; 117(17):4542-51].

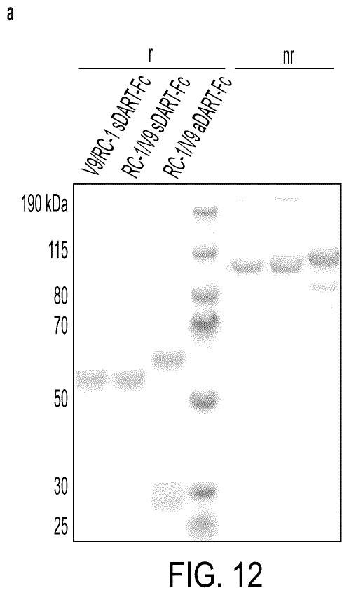

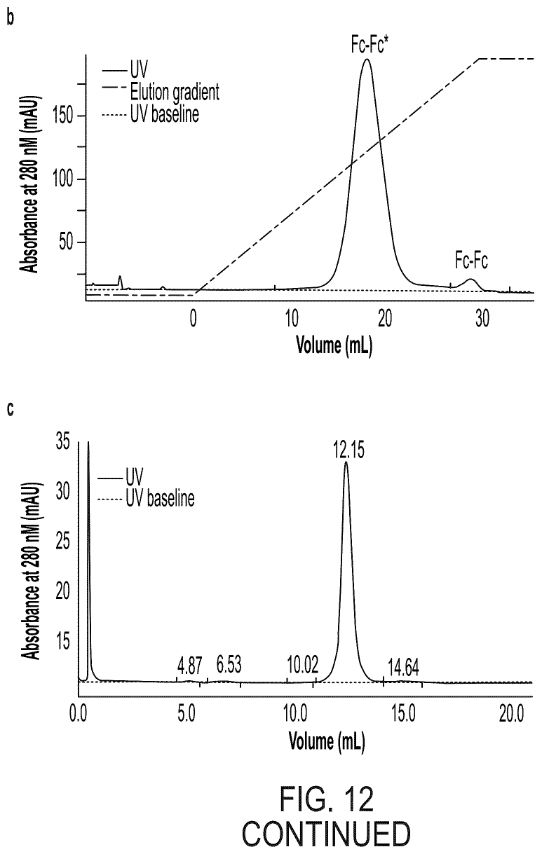

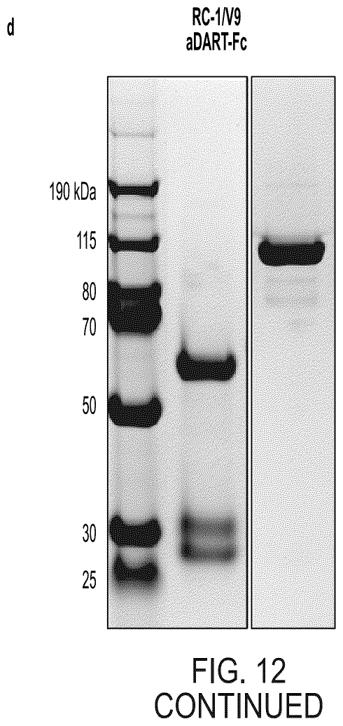

Two sDART-Fc constructs were assembled, and they were named according to the order of V_{L} and V_{H} for each clone RC-1/V9 sDART-Fc and V9/RC-1 sDART-Fc, while only one aDART-Fc construct, RC-1/V9 aDART-Fc, was made (FIG. 12a-d).

DARTs were tested for specificity using flow cytometry and showed specificity only for Siglec-6⁺ and CD3⁺ cell types (FIG. 11b, 12a-d). All DART-Fc T-biAbs demonstrated the ability to activate Jurkat-Lucia NFAT cells, with RC-1/V9 aDART-Fc appearing to be the most potent against Siglec-6⁺ cells without having background NFAT activation in the presence of Siglec-6 cells, which was observed with both sDART-Fc formats (FIG. 11c). The background NFAT activation may be attributable to the sDART-Fc format's tendency towards aggregation (FIG. 12a). RC-1/V9 aDART-Fc was also the most potent in terms of directed T cell lysis of CLL cell lines, with an EC₅₀ of 0.12 pM, a 25-fold increase in potency compared to the RC-1/V9 scFv-Fc (FIG. 11d). In these cytotoxicity assays, the fraction of T cells with both CD69 and CD25 activation markers was larger for the DARTs at 5 pM (˜67%) than it was for the RC-1 clone in scFv-Fc format (27.3%) (FIG. 10b). Against the MEC1-hS6 cell line, the RC-1/V9 aDART-Fc resulted in higher levels of IFN-γ, and IL-2 than the RC-11V9 scFv-Fc, yet only the sDART-Fc formats yielded higher levels of TNF-α than the scFv-Fc, potentially indicating higher risk of CRS [Li J, et al. Science Translational Medicine 2019; 11(508):eaax8861] (FIG. 11e). Against MEC1-002 cells, IL-2 levels were higher with the aDART-Fc than the scFv-Fc, but IFN-γ and TNF-α levels were not significantly higher (FIG. 10b). The aDART-Fc also outperformed both sDART-Fc constructs in terms of IFN-γ and IL-2 induction in the presence of these cells with low Siglec-6 copy number (FIG. 10b). From these data, it was clear that RC-1/V9 aDART-Fc was the DART-Fc candidate with the most therapeutic potential, so the inventors chose to investigate it further.

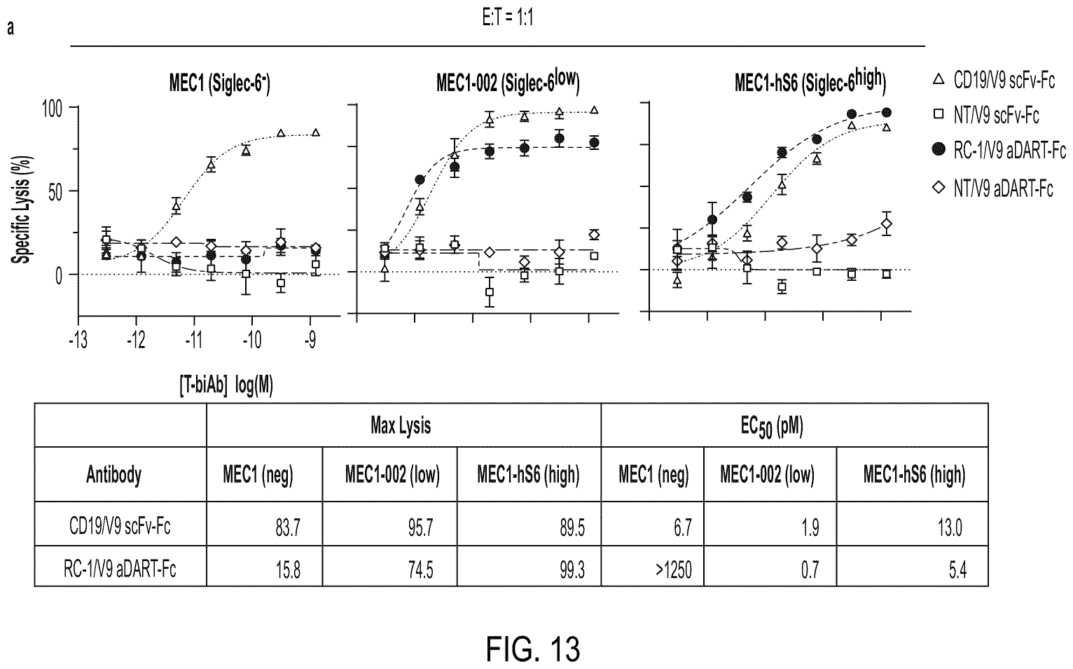

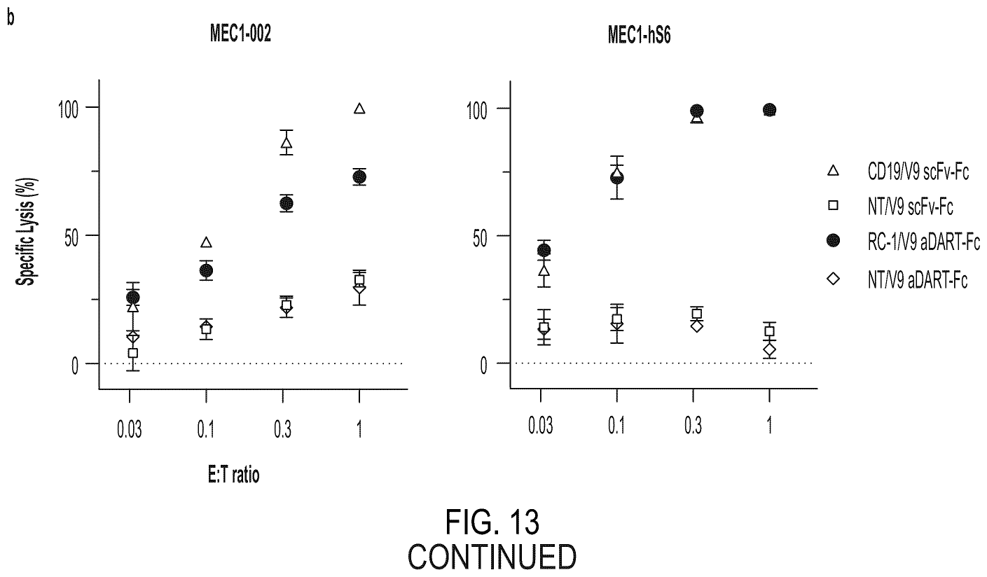

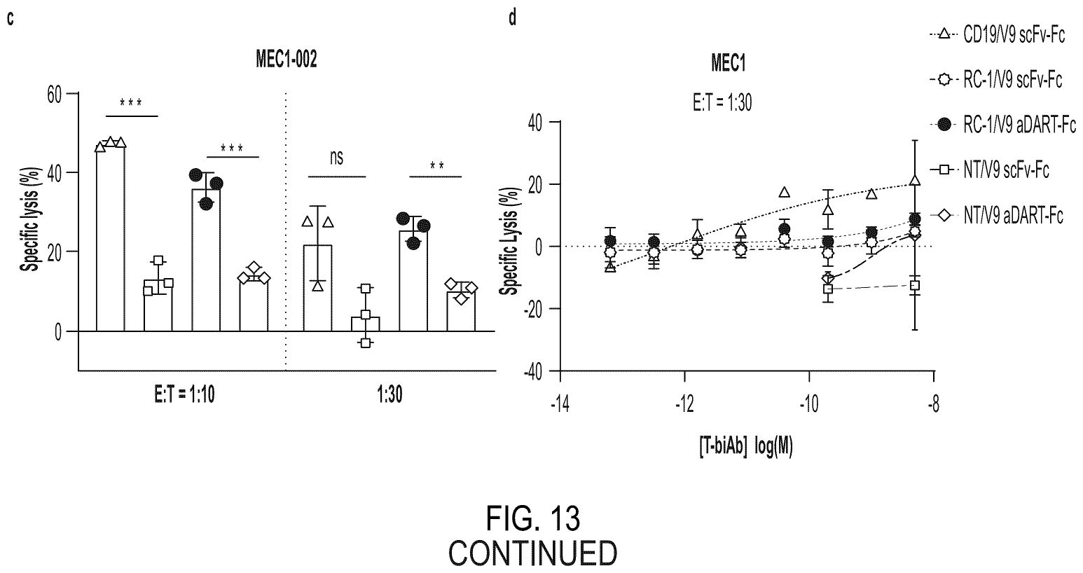

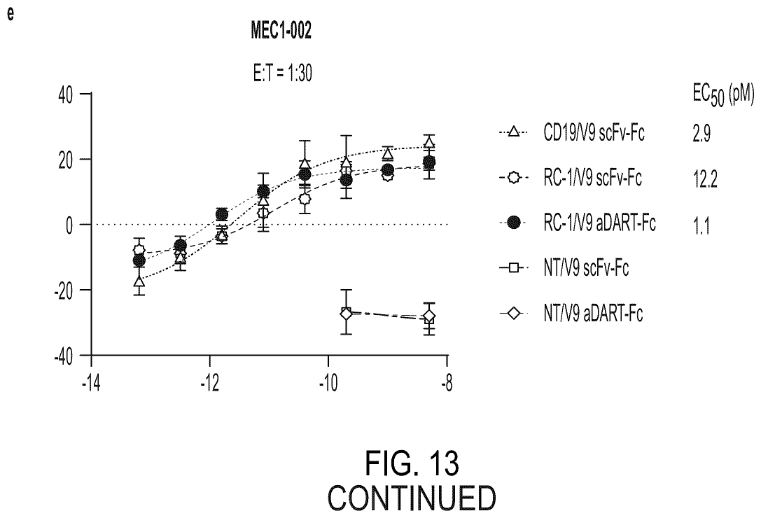

To better predict responses in CLL patients with lower Siglec-6 expression and lower E:T ratios, lysis assays were conducted against MEC1-002 (Siglec-6 antibody binding capacity (ABC I=1,452) and MEC1-hS6 (Siglec-6 ABC=189,390) cell lines, titrating both the T-biAb and the effector cell number (FIG. 13a-c). Specific lysis of MEC1-002 cells occurred down through the low E:T ratio of 1:30, and although the efficacy (maximum lysis) of both Siglec-6 and CD19-targeting T-biAbs decreased at low E:T ratios the potency of RC-1/V9 aDART-Fc (EC₅₀=1.1 pM) was still greater than CD19/V9 scFv-Fc (EC₅₀=2.9 pM) (FIG. 13d-e). These data strongly support RC-1/V9 aDART-Fc for potently and specifically killing target cells and activating T cells even with relatively few effector cells present and low target expression.

### Example 3. Efficacy of T-biAbs Against Primary CLL Cells Ex Vivo

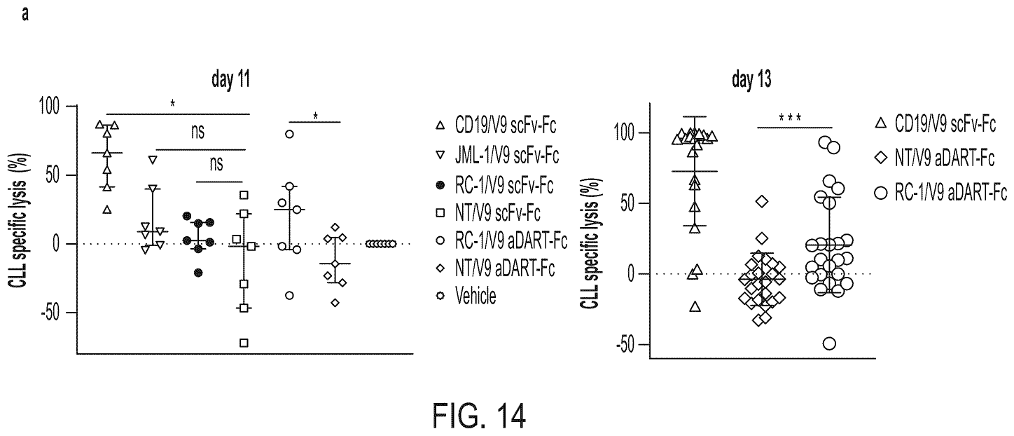

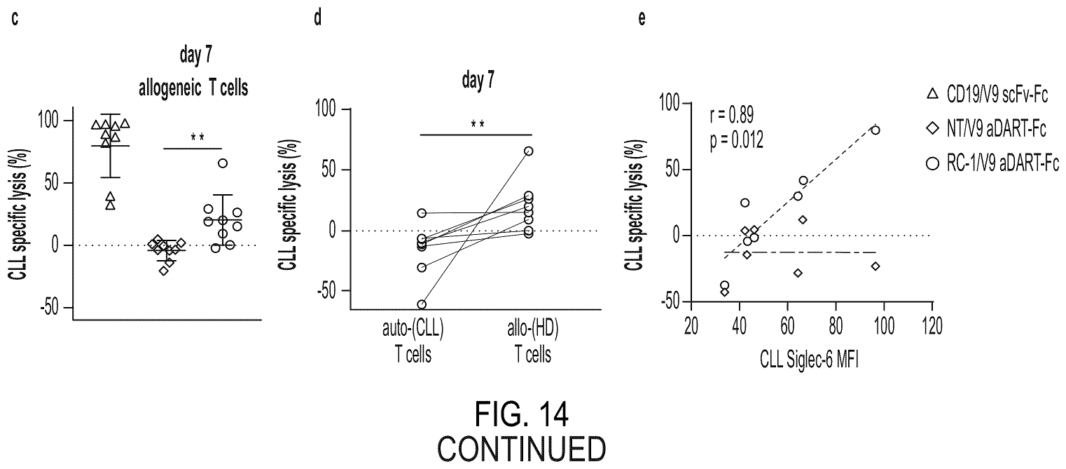

To evaluate the clinical utility of Siglec-6 targeting T-biAbs, they were tested against treatment-naïve CLL patient samples ex vivo. Whole PBMC from CLL patients (n=15. Table 3), with T cell-:CLL cell ratios ranging from 1:15 to 1:200, were cultured with either scFv-Fc or aDART-Fc T-biAbs. After 11 days in culture, the only Siglec-6 T-biAb that resulted in statistically significant lysis of CLL cells was the RC-1/V9 aDART-Fc (median=24.9%); both the JML-1/V9 (median=8.8%) and RC-1/V9 scFv-Fc (median=2.4%) exhibited low target cell lysis (FIG. 14a; Table 4).

TABLE 4
Representative viability and cell count data from CLL cytotoxicity assays
Day 11 treatment-naive CLL cell lysis

| | | | Single cell | | CLL cell |
|---|---|---|---|---|---|
| | | Single cell | viable (% of | CLL cell | viable (% of |
| patient | treatment | viable (#) | single cell) | viable (#) | CLL cell) |
| 2501 | CD19/V9 scFv-Fc | 4102 | 48.7 | 1944 | 34.1 |
| 2501 | RC-1/V9 scFv-Fc | 8756 | 55.2 | 5508 | 46.4 |
| 2501 | JML-1/V9 scFv-Fc | 12753 | 56.7 | 9067 | 51.0 |
| 2501 | NT/V9 scFv-Fc | 9451 | 59.2 | 7712 | 56.2 |
| 2501 | RC-1/V9 aDART-Fc | 12104 | 51.8 | 7759 | 43.7 |
| 2501 | NT/V9 aDART-Fc | 11663 | 58.2 | 10135 | 56.0 |
| 2501 | None | 16584 | 59.9 | 15009 | 58.2 |
| 4973 | CD19/V9 scFv-Fc | 5197 | 36.8 | 28 | 6.4 |
| 4973 | RC-1/V9 scFv-Fc | 6857 | 65.0 | 1241 | 57.0 |
| 4973 | JML-1/V9 scFv-Fc | 4751 | 55.0 | 51 | 28.3 |
| 4973 | NT/V9 scFv-Fc | 10089 | 63.3 | 423 | 36.8 |
| 4973 | RC-1/V9 aDART-Fc | 9706 | 57.4 | 55 | 27.4 |
| 4973 | NT/V9 aDART-Fc | 6656 | 57.5 | 272 | 41.4 |
| 4973 | None | 3802 | 56.6 | 605 | 47.1 |
| 8242 | CD19/V9 scFv-Fc | 1675 | 27.4 | 70 | 6.0 |
| 8242 | RC-1/V9 scFv-Fc | 14285 | 70.2 | 1743 | 45.0 |
| 8242 | JML-1/V9 scFv-Fc | 18631 | 72.3 | 1852 | 48.2 |
| 8242 | NT/V9 scFv-Fc | 10476 | 74.3 | 2849 | 59.5 |
| 8242 | RC-1/V9 aDART-Fc | 12131 | 60.1 | 986 | 32.3 |
| 8242 | NT/V9 aDART-Fc | 5516 | 65.8 | 1594 | 59.1 |
| 8242 | None | 5896 | 64.0 | 1680 | 46.1 |
| 11944 | CD19/V9 scFv-Fc | 11292 | 51.6 | 338 | 6.6 |
| 11944 | RC-1/V9 scFv-Fc | 12992 | 65.1 | 1871 | 28.8 |
| 11944 | JML-1/V9 scFv-Fc | 6615 | 30.3 | 1740 | 13.2 |
| 11944 | NT/V9 scFv-Fc | 11591 | 73.4 | 4857 | 58.1 |
| 11944 | RC-1/V9 aDART-Fc | 10855 | 51.4 | 403 | 6.8 |
| 11944 | NT/V9 aDART-Fc | 5968 | 54.1 | 3127 | 41.6 |
| 11944 | None | 3804 | 40.9 | 2305 | 33.8 |
| 4296 | CD19/V9 scFv-Fc | 5636 | 42.0 | 3985 | 42.2 |
| 4296 | RC-1/V9 scFv-Fc | 7458 | 54.2 | 5638 | 55.2 |
| 4296 | JML-1/V9 scFv-Fc | 7208 | 51.3 | 5089 | 50.4 |
| 4296 | NT/V9 scFv-Fc | 3831 | 60.1 | 2180 | 55.0 |
| 4296 | RC-1/V9 aDART-Fc | 2673 | 59.3 | 1617 | 56.5 |
| 4296 | NT/V9 aDART-Fc | 10956 | 68.9 | 5971 | 61.0 |
| 4296 | None | 6922 | 52.8 | 5780 | 52.7 |
| 10515 | CD19/V9 scFv-Fc | 1490 | 50.4 | 565 | 39.4 |
| 10515 | RC-1/V9 scFv-Fc | 8772 | 67.9 | 4465 | 62.6 |
| 10515 | JML-1/V9 scFv-Fc | 9628 | 72.7 | 4968 | 68.5 |
| 10515 | NT/V9 scFv-Fc | 2253 | 59.2 | 820 | 45.2 |
| 10515 | RC-1/V9 aDART-Fc | 9900 | 75.0 | 5950 | 73.8 |
| 10515 | NT/V9 aDART-Fc | 10557 | 75.8 | 5342 | 68.6 |
| 10515 | None | 7225 | 71.1 | 5823 | 71.2 |
| 11682 | CD19/V9 scFv-Fc | 2616 | 24.6 | 874 | 11.9 |
| 11682 | RC-1/V9 scFv-Fc | 3950 | 54.0 | 1232 | 36.1 |
| 11682 | JML-1/V9 scFv-Fc | 6284 | 51.3 | 2218 | 35.9 |
| 11682 | NT/V9 scFv-Fc | 10383 | 72.4 | 2689 | 50.1 |
| 11682 | RC-1/V9 aDART-Fc | 5304 | 70.7 | 1416 | 49.4 |
| 11682 | NT/V9 aDART-Fc | 10301 | 69.6 | 3111 | 45.1 |
| 11682 | None | 3152 | 41.5 | 1800 | 31.1 |

| | | | | |
|---|---|---|---|---|
| | | Single cell | | CLL cell |
| | Single cell | viable (% of | CLL cell | viable (% of |
| Sample: | viable (#) | single cell) | viable (#) | CLL cell) |

Day 13 treatment-naïve CLL cell lysis

| | | | | |
|---|---|---|---|---|
| 11192_Siglec_E04_021.fcs | 65442 | 58.4 | 25987 | 54.0 |
| 11192_NT_E03_020.fcs | 66160 | 53.4 | 29594 | 59.6 |
| 11192_untreated_E01_018.fcs | 56497 | 47.5 | 28151 | 60.4 |
| 11192_19, 2f, 3_E02_019.fcs | 19888 | 17.7 | 650 | 1.9 |
| 11627_Siglec_C04_013.fcs | 12931 | 47.3 | 2870 | 52.4 |
| 11627_NT_C03_012.fcs | 21713 | 61.0 | 3466 | 56.0 |
| 11627_Untreated_C01_011.fcs | 35557 | 57.4 | 4222 | 49.5 |
| 12033_Siglec_D04_017.fcs | 63143 | 21.2 | 10338 | 54.3 |
| 12033_NT_D03_016.fcs | 82674 | 50.0 | 22783 | 81.8 |
| 12033_Untreated_D01_014.fcs | 65428 | 26.1 | 13259 | 68.7 |
| 12033_19, 2f, 3_D02_015.fcs | 40119 | 17.4 | 277 | 3.6 |
| 12202_Siglec_B04_010.fcs | 65259 | 44.5 | 20886 | 70.4 |
| 12202_NT_803_009.fcs | 56062 | 69.3 | 28389 | 86.7 |
| 12202_Untreated_B01_007.fcs | 53714 | 54.8 | 40240 | 79.1 |
| 12202_19, 2f, 3_B02_008.fcs | 52339 | 36.6 | 62 | 0.7 |
| 5539 PBMC_Siglec_E01_010.fcs | 78041 | 50.2 | 15746 | 39.4 |
| 5539 PBMC_NT_D01_009.fcs | 49424 | 70.0 | 28901 | 74.1 |
| 5539 PBMC_Untreated_B01_007.fcs | 32209 | 77.9 | 23699 | 80.4 |
| 5539 PBMC_19, 2f, 3_C01_008.fcs | 125207 | 60.1 | 767 | 4.0 |
| 8289 PBMC_Siglec_E02_014.fcs | 62913 | 54.1 | 19153 | 66.6 |
| 8289 PBMC_NT_D02_013.fcs | 45972 | 68.6 | 35554 | 82.9 |
| 8289 PBMC_Untreated_B02_011.fcs | 16263 | 68.5 | 9865 | 71.1 |
| 8289 PBMC_19, 2f, 3_C02_012.fcs | 45523 | 42.2 | 203 | 2.2 |
| 11597 PBMC_Siglec_E03_018.fcs | 69413 | 56.9 | 42424 | 71.9 |
| 11597 PBMC_NT_D03_017.fcs | 15174 | 43.2 | 13064 | 43.0 |
| 11597 PBMC_Untreated_B03_015.fcs | 18678 | 48.4 | 16717 | 48.4 |
| 11597 PBMC_19, 2f, 3_C03_016.fcs | 65924 | 62.2 | 30858 | 59.8 |
| 4086 PBMC_Siglec_E03_017.fcs | 4413 | 6.4 | 3029 | 6.4 |
| 4086 PBMC_NT_D03_016.fcs | 182665 | 78.0 | 160656 | 83.4 |
| 4086 PBMC_Untreated_B03_014.fcs | 171000 | 65.5 | 133048 | 68.4 |
| 4086 PBMC_19, 2f, 3_C03_015.fcs | 203183 | 72.2 | 143426 | 76.1 |
| 5407 PBMC_Siglec_E02_013.fcs | 198408 | 58.0 | 174709 | 63.2 |
| 5407 PBMC_NT_D02_012.fcs | 366499 | 80.3 | 341752 | 82.4 |
| 5407 PBMC_Untreated_B02_010.fcs | 296113 | 62.6 | 265734 | 63.3 |
| 5407 PBMC_19, 21, 3_C02_011.fcs | 218573 | 47.4 | 131963 | 43.1 |
| 5508 PBMC_Siglec_E01_009.fcs | 137437 | 61.3 | 76848 | 63.4 |
| 5508 PBMC_NT_D01_008.fcs | 317754 | 79.9 | 210636 | 85.7 |
| 5508 PBMC_Untreated_B01_007.fcs | 228296 | 78.7 | 164775 | 81.6 |

Day 7 treatment-naïve CLL cells + allogeneic T cells

| | | | | |
|---|---|---|---|---|
| DAY7 Siglec Mix 5539_UT_B04_019.fcs | 105579 | 64.1 | 25861 | 55.2 |
| DAY7 Siglec Mix 5539_19_3_C04_020.fcs | 1541 | 1.1 | 739 | 1.7 |
| DAY7 Siglec Mix 5539_NT_D04_021.fcs | 78467 | 58.9 | 26836 | 62.8 |
| DAY7 Siglec Mix 5539_SIG_E04_022.fcs | 126773 | 61.0 | 20017 | 46.8 |
| DAY7 Siglec Mix 8289_UT_B05_023.fcs | 145145 | 74.5 | 68998 | 86.9 |
| DAY7 Siglec Mix 8289_19_3_C05_024.fcs | 99437 | 49.5 | 3163 | 11.3 |
| DAY7 Siglec Mix 8289_NT_D05_025.fcs | 73532 | 67.7 | 29722 | 82.6 |
| DAY7 Siglec Mix 8289_SIG_E05_026.fcs | 135502 | 67.7 | 37906 | 69.4 |
| DAY7 Siglec Mix 11597_UT_B06_027.fcs | 141951 | 75.2 | 60816 | 82.6 |
| DAY7 Siglec Mix 11597_19_3_C06_028.fcs | 78866 | 40.4 | 1326 | 3.5 |
| DAY7 Siglec Mix 11597_NT_D06_029.fcs | 97317 | 63.8 | 59682 | 85.4 |
| DAY7 Siglec Mix 11597_SIG_E06_030.fcs | 187476 | 71.5 | 65263 | 82.4 |
| 4086 Mix_Siglec_E06_029.fcs | 130752 | 62.5 | 52314 | 64.3 |
| 4086 Mix_NT_D06_028.fcs | 41004 | 39.6 | 20051 | 46.7 |
| 4086 Mix_Untreated_B06_026.fcs | 43522 | 63.8 | 17685 | 65.4 |
| 4086 Mix_19, 2f, 3_C06_027.fcs | 97715 | 50.4 | 2554 | 7.0 |
| 5407 Mix_Siglec_E04_021.fcs | 158203 | 76.8 | 89572 | 79.5 |
| 5407 Mix_NT_D04_020.fcs | 138000 | 72.7 | 106356 | 85.8 |
| 5407 Mix_Untreated_B04_018.fcs | 240469 | 85.0 | 163361 | 87.7 |
| 5407 Mix_19, 2f, 3_C04_019.fcs | 168877 | 62.4 | 84880 | 59.2 |
| 5508 Mix_Siglec_E05_025.fcs | 50729 | 55.5 | 5941 | 31.7 |
| 5508 Mix_NT_D05_024.fcs | 57524 | 73.6 | 33394 | 85.4 |
| 5508 Mix_Untreated_B05_022.fcs | 139944 | 88.4 | 59214 | 92.8 |
| 5508 Mix_19, 2f, 3_C05_023.fcs | 111327 | 53.3 | 7508 | 16.6 |

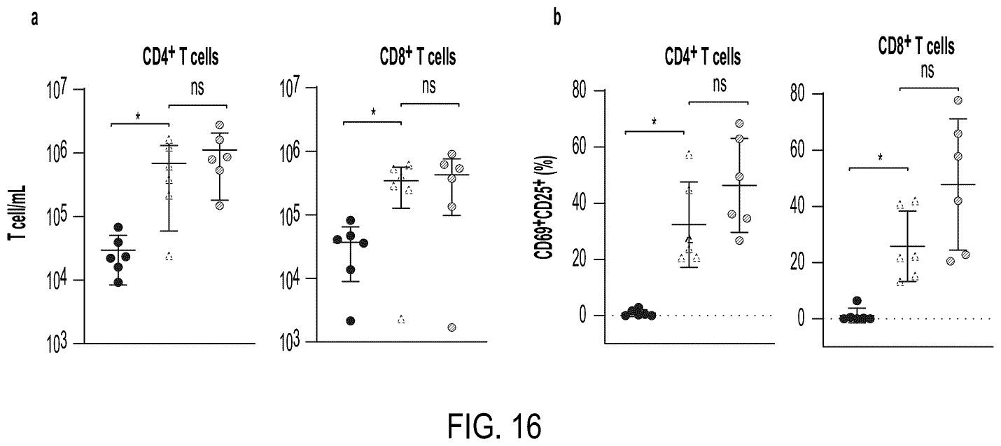

Thus, these data agree with the findings from experiments on cell lines, that the aDART-Fc is the most effective T-biAb format for antibody clones targeting a membrane distal epitope on Siglec-6. An expanded CLL cohort (n=38) was tested against RC-1/V9 aDART-Fc in ex vivo cultures, and cell lysis was not significant until day 10, suggesting that T cell expansion may be a factor for Siglec-6×CD3 T-biAbs (FIG. 14b, 15a). T cell expansion was evaluated at day 9 of ex vivo culture and this verified that both CD4 and CD8⁺ T cell subsets proliferated and upregulated activation markers in response to T-biAb treatment, indicating that activation and expansion are occurring by this time point (FIG. 16a-b).

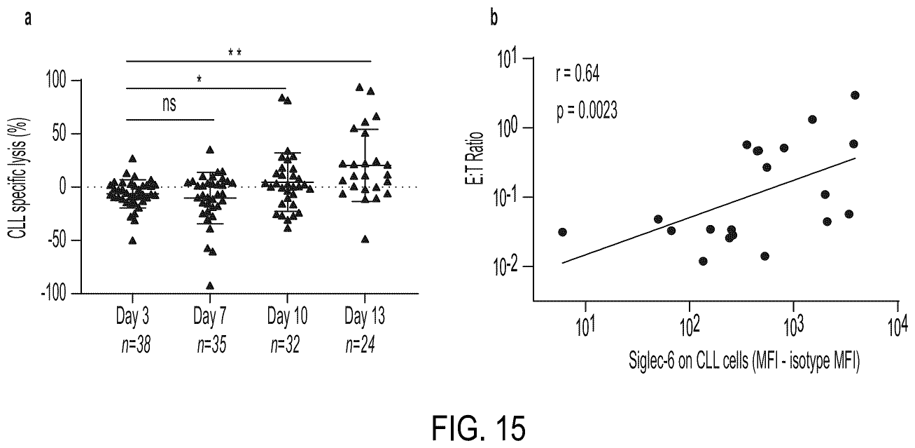

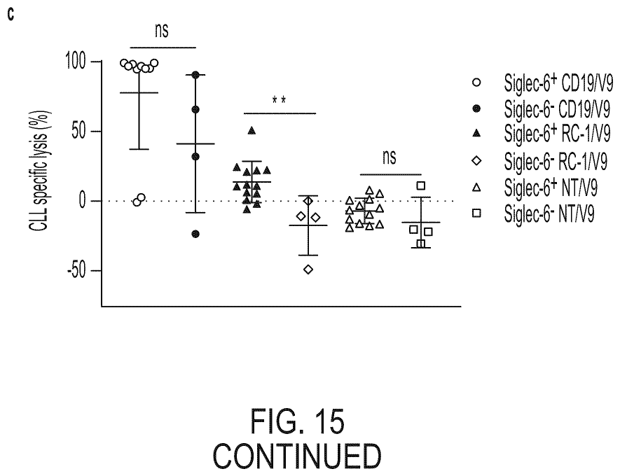

To determine whether CLL lysis is dependent on T cell number and/or quality, allogeneic PBMC from a healthy donor were cultured with primary CLL cells at an E:T ratio of 1:1. T-biAb-mediated specific lysis was significant at day 7 of this co-culture, and higher in comparison to an autologous culture run in parallel, suggesting that both low E:T ratio and T cell dysfunction in treatment-naïve CLL samples [Ramsay A G, et al. J Clin Invest 2008:118(7):2427-37] play a role in limiting efficacy of Siglec-6×CD3 T-biAbs (FIG. 14c-d). As expected, RC-1/V9 aDART-Fc-mediated primary CLL cell lysis correlated with Siglec-6 expression at baseline (FIG. 14e). Interestingly, there was a correlation between Siglec-6 expression and E:T ratio at baseline indicating a potential relationship between immune activation (exemplified by expansion of T cells) and expression of Siglec-6 in B cells (FIG. 14b, FIG. 15b). When CLL patients were grouped based on Siglec-6 expression, specific lysis was higher in the Siglec-6 group when treated with RC-1/V9 aDART-Fc (FIG. 15c). The increased lysis of Siglec-6⁺ CLL cells by RC-1/V9 aDART-Fc was dependent on Siglec-6 expression, and not E:T ratio, because specific lysis did not increase between Siglec-6⁺ and Siglec-6⁺ primary CLL cell samples with the CD19/V9 scFv-Fc.

Since most patients with CLL receive BTKis such as ibrutinib or acalabrutinib as a frontline treatment, the inventors assessed Siglec-6 expression on a cohort of patients (n=7) before (pre-BTKi) and while undergoing (on-BTKi) treatment (FIG. 19a-c). The inventors found that Siglec-6 expression does not change on-BTKi, although the E:T ratio increased as anticipated. RC-1/V9 aDART-Fc cytotoxicity assays with BTKi-treated patients revealed significant levels of CLL cell lysis at day 3 and day 11 of ex vivo culture, suggesting that Siglec-6 directed therapies will maintain efficacy in combination with BTKi (FIG. 19d, e, Table 5).

TABLE 5
BTKi-treated CLL patient characteristics

| | % | | | | | | | | | | | |
|---|---|---|---|---|---|---|---|---|---|---|---|---|
| | Siglec-6+ | | | | | | | Treatment | | IGHV | | |
| Sample | (live CLL | Siglec-6 | | | | Timepoint | Age/ | history | Rai | Status | | ALC, |
| ID | cells) | MFI | E: | T | BTKi | (months) | Gender | (TN/RR) | Stage | (M/UM) | Cytogenetics | k/μ |
| 9746 | 4 | 141.8 | 1: | 55.1 | PRE | 0 | 64/M | TN | 3 | U | del13q | 93.4 |
| 12032 | 67.6 | 1943 | 1: | 3.1 | acalabrutinib | 28 | 67/M | TN | 3 | U | del13q, | 3.6 |
| | | | | | | | | | | | NOTCH1 | |
| 8330 | 1.69 | 157.2 | 1: | 90.5 | PRE | 0 | 53/F | RR | 3 | M | del13q | 178.1 |
| 11913 | 95.5 | 3154 | 1: | 4.0 | acalabrutinib | 48 | 57/F | RR | 3 | M | del13q | 7.6 |
| 9132 | 0.38 | 55.6 | 1: | 22.5 | PRE | 0 | 65/M | RR | 4 | U | del11q, | 120 |
| | | | | | | | | | | | del14q | |
| 9778 | 2.26 | 49 | 1: | 23.3 | acalabrutinib | 6 | 65/M | RR | 4 | U | del11q, | 129.8 |
| | | | | | | | | | | | del14q | |
| 9450 | 0.62 | 69.9 | 1: | 199.0 | PRE | 0 | 68/F | TN | 3 | U | del13q, | 225.8 |
| | | | | | | | | | | | del17p | |
| 9939 | 8.18 | 125.7 | 1: | 26.0 | acalabrutinib | 6 | 68/F | TN | 3 | U | del13q, | 36.6 |
| | | | | | | | | | | | del17p | |
| 6811 | 1.57 | 98 | 1: | 33.7 | PRE | 0 | 70/M | TN | 4 | M | del13q, | 86.3 |
| | | | | | | | | | | | del17p | |
| 7791 | 13.5 | 93.9 | 1: | 21.2 | ibrutinib | 6 | 71/M | TN | 4 | M | del13q, | 65.8 |
| | | | | | | | | | | | del17p | |
| 7786 | 2.87 | 300.7 | 1: | 64.3 | PRE | 0 | 66/M | TN | 1 | ND | del13q, | 257.5 |
| | | | | | | | | | | | Tri12 | |
| 9153 | 18.4 | 104 | 1. | 3.3 | ibrutinib | 24 | 68/M | TN | 1 | ND | del13q, | 5.3 |
| | | | | | | | | | | | Tri12 | |
| 7562 | 0.68 | 70.6 | 1: | 28.4 | PRE | 0 | 66/F | RR | 4 | U | del11q, | 155.5 |
| | | | | | | | | | | | del13q | |
| 8156 | 3.87 | 34 | 1: | 5.8 | ibrutinib | 12 | 67/F | RR | 4 | U | del11q, | 6.71 |
| | | | | | | | | | | | del13q | |

Timepoint = time since starting treatment; MFI = mean fluorescence intensity; IGHV = immunoglobulin heavy chain; status: m = mutated, u = unmutated; ALC = absolute lymphocyte count.

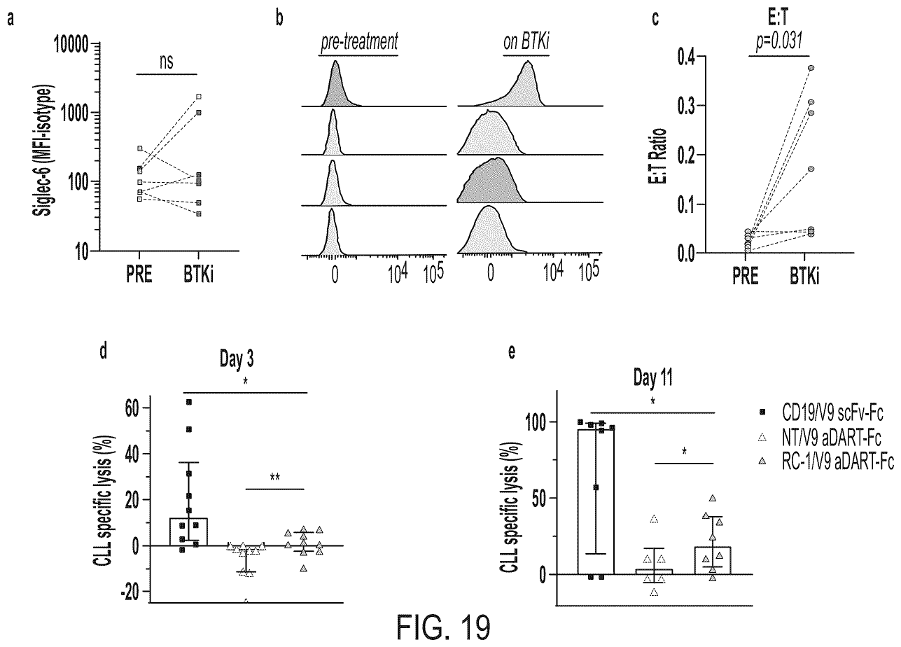

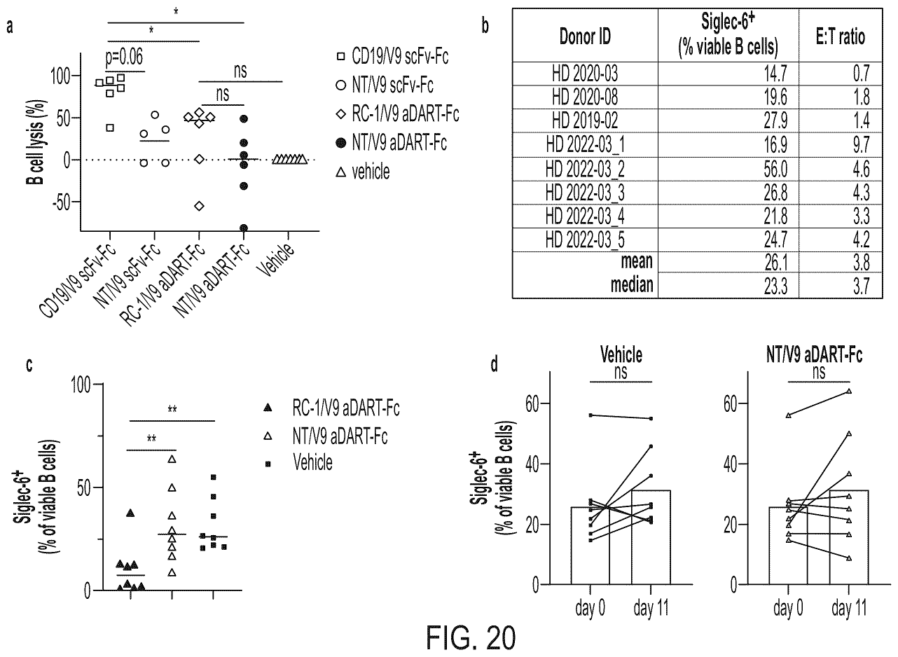

Although T-biAbs targeting CD19 were more effective than those targeting Siglec-6, CD19 is a marker found on all healthy B cells, while Siglec-6 expression is restricted to a subset of activated B cells. In ex vivo cultures of healthy donor PBMC (using conditions identical to the CLL cytotoxicity assays), the CD19/V9 scFv-Fc killed a larger fraction of healthy B cells at the day 11 time point compared to the RC-1/V9 aDART-Fc and the non-targeting NT/V9 aDART-Fc (FIG. 20a). Notably, there was no significant difference between the latter two, despite the fact that the E:T ratio in these healthy donor PBMC was up to 1,000-fold higher than in the CLL PBMC (FIG. 20a, b). Since a subset of healthy donor B cells express Siglec-6 (median=23.3%), some lysis was still observed from RC-1/V9 aDART-Fc. As expected, the fraction of viable healthy B cells expressing Siglec-6 was lower in the RC-1/V9 aDART-Fc treatment group than the NT/V9 aDART-Fc or vehicle control groups, suggesting the Siglec-6⁺ fraction of healthy B cells were selectively depleted (FIG. 20c). Since the overall percent lysis was higher than expected based on the initial size of the Siglec-6⁺ fraction of B cells, the inventors wondered whether the culture conditions facilitated the outgrowth of activated B cells (which encompasses the Siglec-6 fraction of healthy B cells). The fraction of B cells expressing Siglec-6 increased slightly over the course of these experiments in both the vehicle and NT/V9 aDART-Fc treatment groups, yet the difference was not significant (FIG. 20d). These data collectively indicate that Siglec-6-targeting T-biAbs eliminate Siglec-6-expressing CLL and healthy B cells but spare the bulk of healthy B cells.

### Example 5. Investigating the Basis for Differential Activity of T-biAb

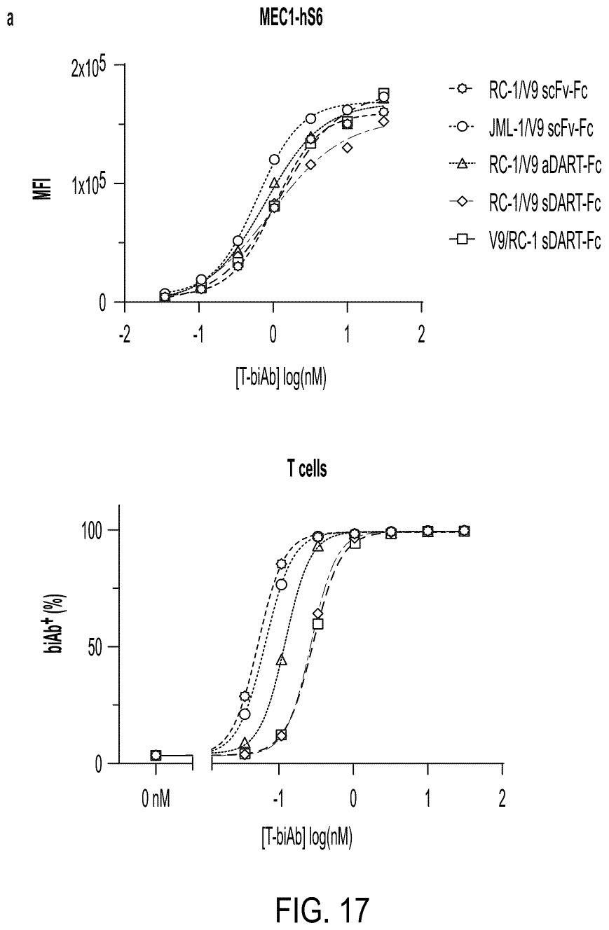

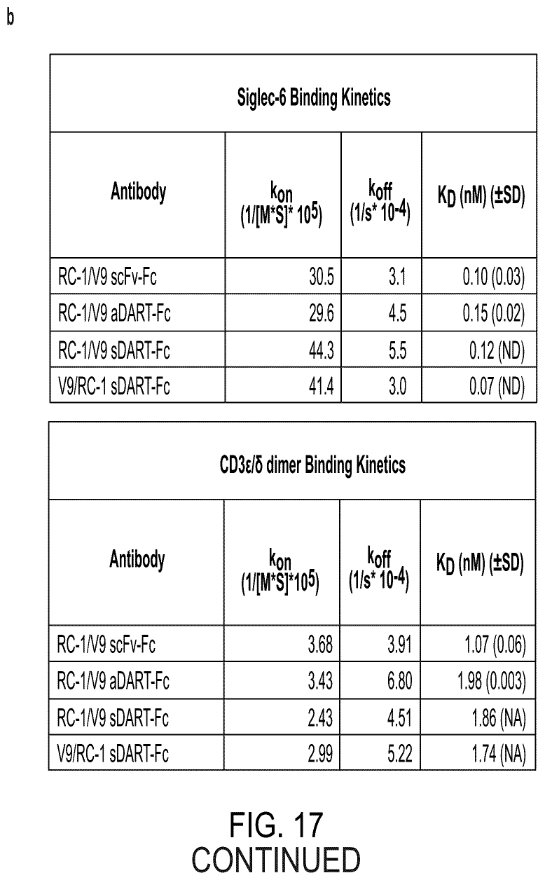

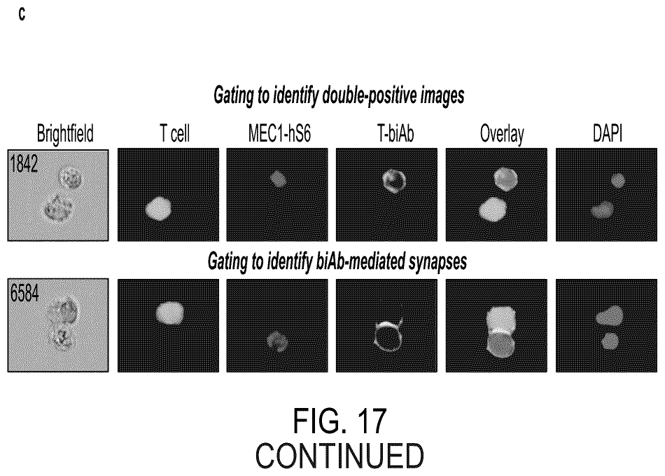

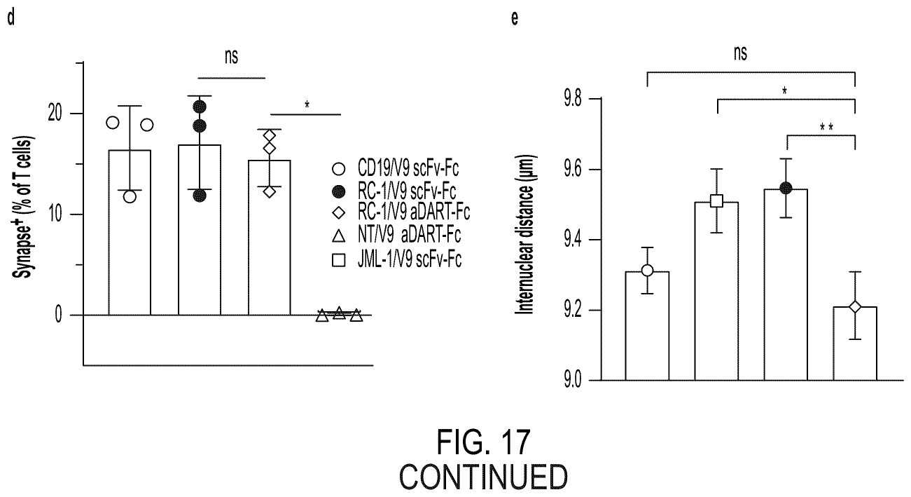

To determine whether T-biAb format affected the affinity to Siglec-6 or CD3 on the cell surface, T-biAbs were titrated and used to stain cells for flow cytometry (FIG. 17a). In terms of Siglec-6 binding, RC-1/V9 aDART-Fc (apparent K_{d}=0.79 nM) fell between the scFv-Fcs JML-1/V9 (apparent K_{d}=0.61 nM) and RC-1/V9 scFv-Fc (apparent K_{d}=1.1 nM). Against T cells, CD3 binding appeared to be more potent by a factor of two for the scFv-Fc than for the aDART-Fc. To quantify the affinities more accurately, the inventors used SPR which revealed that the affinity to both Siglec-6 and CD3 was slightly higher for the RC-1/V9 scFv-Fc than the RC-1/V9 aDART-Fc (FIG. 17b). However these differences were subtle, and both in the same direction, thus the format change did not reverse the target preference ratio. Therefore, affinity alone cannot account for the 10-fold higher potency of the DART-Fc over the scFv-Fc in lysis assays.

To quantify characteristics of the synapse, the inventors used multispectral fluorescence imaging. To control for any subtle differences in binding orientation of different clones, the inventors focused on the RC-1 clone, and compared the scFv-Fc and aDART-Fc formats, since they showed 25-fold difference in cytotoxicity EC₅₀ (FIG. 11d), and the aDART-Fc lacked the non-specific background attributed to sDART-Fc aggregation (FIGS. 11c, 12a). Target (MEC1-hS6) and effector (Jurkat T cells) cells were co-cultured with T-biAb, fixed, and analyzed. Initial gating to identify double-positive images containing both cell types lacked the stringency to selectively identify synapses (FIG. 17c, top). However, additional gating to require overlap between target and effector cells, as well as colocalization of T-biAb at the synapse clearly delineated images with true T-biAb-mediated synapses (FIG. 17c, bottom). The percentage of T cells forming synapses were similar for CD19/V9 scFv-Fc, RC-1/V9 scFv-Fc. and RC-1/V9 aDART-Fc, confirming that at a saturating concentration all constructs are equally adept at forming synapses (FIG. 17d).

To investigate the initial hypothesis, namely that the DART-Fc—would result in a shorter synapse, the inventors measured the distance between the nuclei in each synapse⁺ cell pair, as a proxy for synapse length (FIG. 11e). This revealed that the RC-1/V9 aDART held cells closer together, than the RC-1/V9 scFv-Fc. Accordingly, the improvement in efficacy of the DART-Fc may be attributed to the shorter or more rigid synapse formed when compared to an scFv-Fc T-biAb.

### Example 5. RC-1/V9 aDART-Fc Activity In Vivo

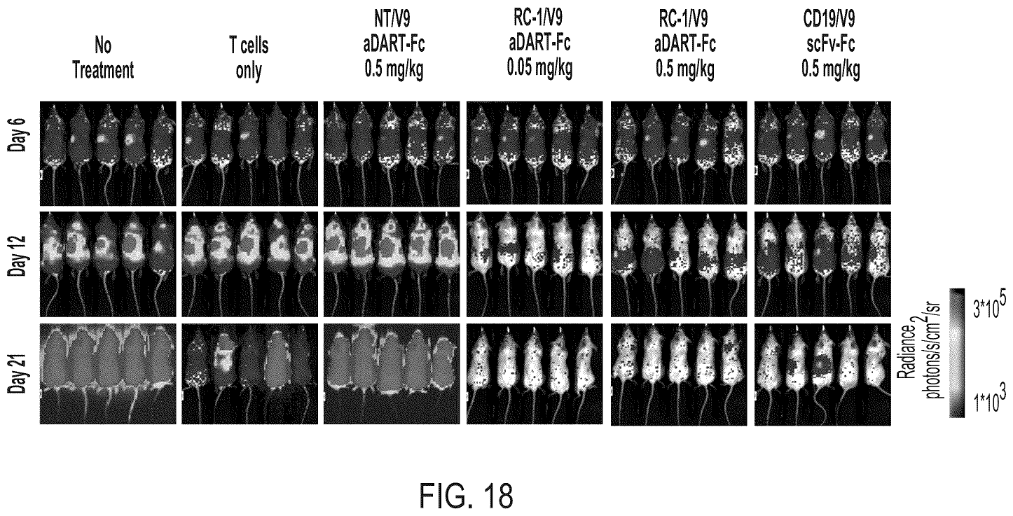

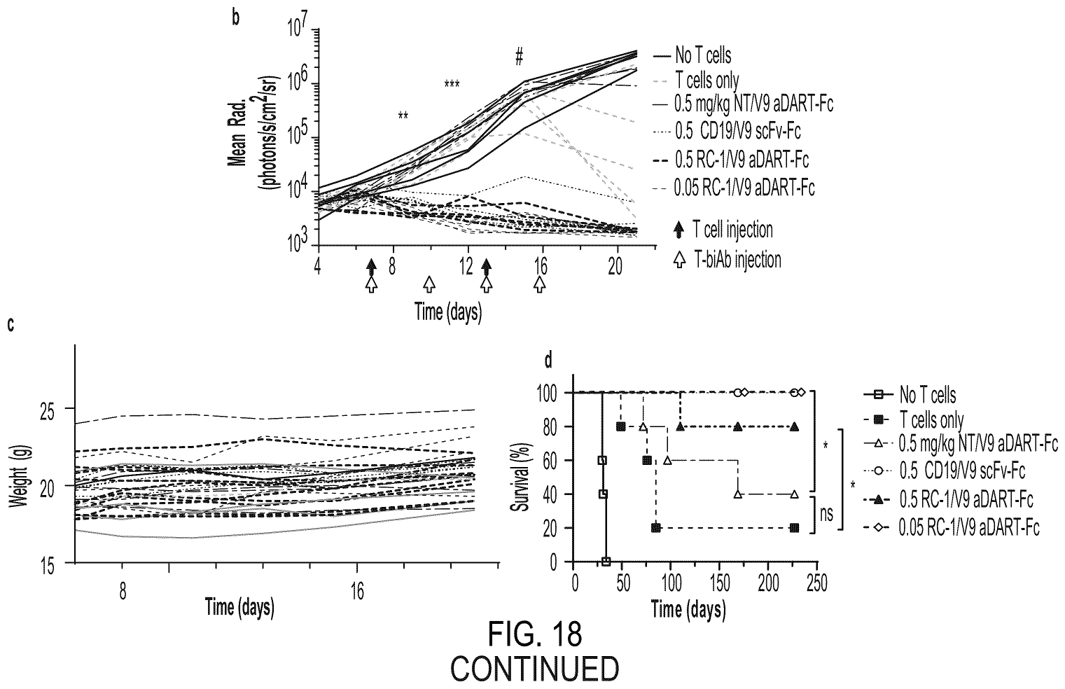

To characterize the function of the RC-1/V9 aDART-Fc in vivo the inventors generated a MEC1-fLuc-Siglec-6 xenograft mouse model in NOD/SCID/IL2Rγnull (NSG) mice, as a systemic cell line-derived xenograft (CDX) mouse model of CLL. After the first treatment with low dose (0.05 mg/kg) or high dose (0.5 mg/kg) RC-1/V9 aDART-Fc, the leukemic burden was significantly reduced compared to the NT/V9 aDART-Fc (FIG. 18a, b). By day 21, the MEC1-hS6 cells were cleared in mice treated with Siglec-6- and CD19-targeting T-biAbs. No signs of treatment-related toxicity or decrease in bodyweight were observed over the course of the study (FIG. 18c). By day 21 and later the tumor burden decreased in some T cell only and NT/V9 aDART-Fc treated mice which was, attributable to allogeneic killing—a common phenomenon in CDX models [Kovalevsky, supra]. Despite this non-specific T cell activity, the 0.05 mg/kg dose of RC-1/19 aDART-Fc significantly extended the overall survival compared to the negative controls, similar to the CD19/V9 scFv-Fc treatment (FIG. 18d). Surprisingly, no dose response was observed between the 0.05 and 0.5 mg/kg doses of RC-1/19 aDART-Fc. The only relapse observed was in the high dose group, suggesting that 0.5 mg/kg may be above the optimal activity window for the three-component binding model, resulting in a hook effect [Douglass E F, Jr., et al. J Am Chem Soc 2013:135(16):6092-9].

A follow-up pharmacokinetic study was performed to assess in vivo half-life of the Siglec-6- and CD19-targeting T-biAbs.

NSG mice were preconditioned with 250 μL of human serum by intraperitoneal (i.p.) injection 24 h prior to injection of 50 μg (2.5 mg/kg) RC-1/V9 aDART-Fc by intravenous (i.v.) or i.p. injection. Blood was collected from the tail vein at various time points over the course of 22 days, and T-biAbs in the serum were detected by ELISA, which consisted of capturing recombinant CD3EB dimer and detecting with an anti-human Fc secondary. The values in the table are the averages and standard deviations for 3 (i.v. +serum cage) to 4 (all others) mice per treatment group. AUC=area under curve. C_{L}=clearance. V_{SS}=steady state volume of distribution.

As shown in Table 6, the results revealed that the half-life of the aDART-Fc in NSG mice is 7.9 days after conditioning with human serum and i.v. injection Therefore RC-1/V9 aDART-Fc would have accumulated over the course of the study with twice-weekly dosing, reaching 100 nM in the blood, ˜800-fold higher than the EC₅₀ for lysis in vitro. (Table S5). Collectively, these results demonstrate that a Siglec-6×CD3 T-biAb in aDART-Fc format is highly potent at clearing CLL in vivo, has long circulatory half-life, and warrants further preclinical studies.

TABLE 6
Pharmacokinetic parameters of RC-1/V9 aDART-Fc.

| | Condi- | Half- | AUC | CL | Vₛₛ |
|---|---|---|---|---|---|
| Route | tioning | life (h) | (mg/mL × h) | (mL/h/kg) | (mL/kg) |
| IV | — | 170 ± 67 | 4.9 ± 0.5 | 0.51 ± 0.06 | 120 ± 40 |
| IV | human | 190 ± 17 | 4.8 ± 0.4 | 0.53 ± 0.05 | 117 ± 18 |
| | serum | | | | |
| IP | — | 254 ± 114 | 6.8 ± 2.3 | 0.40 ± 0.14 | |
| IP | human | 218 ± 59 | 6.2 ± 1.4 | 0.42 ± 0.10 | |
| | serum | | | | |

The values in the table are the averages and standard deviations for 3 to 4 mice per treatment group.

### Example 6. Materials and Methods

Design and cloning of antigens and antibodies:

The extracellular domains from human Siglec-6 (accession 043699-1, amino acids (aa) 27-347) and Macaca mulatta (rhesus macaque) Siglec-6 (UniProt entry A0A1 D5QH63, aa 16-333) were each cloned into pCEP4 at the C-terminus of human IgG1 Fc (aa 99-329). Non-Fc containing Siglec-6 constructs were generated by cloning the V-type and C2-type I Ig-like domains of Siglec-6, aa 28-235 (Siglec-6 VC₂₈₋₂₃₅) from a gene fragment (Twist Bioscience), with a 6×His tag encoded at the C-terminus, into the pHL-sec vector (Addgene plasmid #99845) using Age and KpnI restriction sites [Aricescu A R et al. Acta Crystallogr D Biol Crystallogr 2006; 62(Pt 10): 1243-50] Fabs.

Human Fab-encoding sequences selected by phage display were cloned from pC3C into pET11a, using the restriction enzyme Slit (New England Biolabs) [Kwong K Y, Rader C. Curr Protoc Protein Sci 2009; Chapter 6: Unit 6 10; Stahl S J, et al. J Mol Biol 2010; 397(3):697-708]. T-biAbs in scFv-Fc format. JML-1 scFv and RC-1 scFv gene fragments were synthesized as gBlocks (Integrated DNA Technologies) in the following format: V_{H}-(Gly₄ Ser)₃ linker-V_{L}. These scFv fragments were cloned into a pCEP4 vector with an Fc domain containing knob mutations, S354C and T366W previously generated in the inventors' lab using KpnI and XhoI restriction enzymes (New England Biolabs). The humanized variant (V9) of the anti-CD3 mAb UCHT1 was incorporated in the Fc-h arm as previously described [Qi 2018, supra]. The anti-CD19 (clone 21D4)/V9 scFv-Fc and anti-HER2 (trastuzumab clone)/V9 scFv-Fc previously reported were used as the positive and negative (non-targeting, NT) controls, respectively [Qi 2018, supra; Robinson, supra]. T-biAbs in DART-Fc format. The Fc component of the DART formats employed gene fragments containing L234A, L235A, P329G mutations to eliminate all Fc-gamma receptor interactions [Schlothauer T, et al. Protein Eng Des Sel 2016; 29(10):457-66]. The knobs-into-holes mutations used in DART-Fc were identical to the scFv-Fc but the holes-containing half also possessed Fc-star mutations H435Y and R436F to allow for complete heterodimer separation [Jendeberg L, et al. J Immunol Methods 1997; 201(1):25-34]. Symmetric DART (sDART)-Fc constructs were composed of 2 chains, have variable domains each fused to an Fc domain similar to the CDH3×CD3 DART-Fc PF-06671008, and are named in order that the clones appear on the holes-containing arm. V9/RC-1 sDART-Fc: V9ᵥₖ-G₃SG₄-RC-1ᵥₕ-G-CPPCP-Fc-star-ₕₒₗₑₛ and RC-1ᵥₖ-G₄SG₄-V9ᵥₕ-G-CPPCP-Fc-_{knobs}. RC-1/V9 sDART-Fc: RC-1ᵥₖ-G₃SG₄-V9ᵥₕ-G-CPPCP-Fc-star-ₕₒₗₑₛ and V9ᵥₖ-G₄SG₄-RC-1ᵥₕ-G-CPPCP-Fc-_{knobs}. Asymmetric DART (aDART)-Fc is composed of 3 chains requiring coiled-coil motifs for associating the 2 targeting domains, similar to the CD19×CD3 DART-Fc duvortuxizumab [Liu L, et al. Clin Cancer Res 2017; 23(6):1506-18; World Health Organization, Drug Information 2016, vol. 30, 4 WHO Drug Information 2016; 30(4):545-604]. Chain 1: RC-1ᵥₖ-G₃SG₄-V9ᵥₕ-ASTK-E-coil-G₃-Fc_{knobs}; chain 2: V9ᵥₖ-G₄SG₄-RC-1ᵥₕ-ASTK-K-coil; chain 3: Fcₕₒₗₑₛ. For the negative (non-targeting, NT) aDART-Fc, a human phage-display-derived anti-tetanus toxoid clone TT11 [Kwong 2008 supra] was used to replace RC-1. Amino acid sequences are provided in the Sequence Listing.

### Expression and Purification of Antigens and Antibodies

Siglec-6 proteins. Recombinant Fc-siglec fusion proteins for human Siglec-6 and rhesus Siglec-6 were generated in house using FreeStyle 293-F cells and purified via Protein A affinity chromatography [Kovalovsky, supra]. Other siglec-Fc fusions were purchased from R&D Systems. Non-Fc containing Siglec-6 constructs were expressed in HEK293S GnTi⁻ cells, purified via immobilized metal affinity chromatography (IMAC via HisTrap HP, Cytiva, formerly GE Healthcare Life Sciences) [Aricescu supra; Ereño-Orbea J. et al. J Vis Exp 2018; 137:57750]. For deglycosylation, Siglec-6 glycoprotein at 0.5 mg/mL was subjected to enzymatic treatment using 22,500 U/mL Remove-IT PNGase F (New England Biolabs). After overnight incubation at 37° C., chitin magnetic beads (New England Biolabs) were used to remove the enzyme, and the deglycosylated protein was concentrated for HDX analysis. Fabs. Human Fab-encoding sequences selected by phage display were expressed and purified from E. coil using IMAC and CaptureSelect Kappa or CH1 resin (both from Thermo Fisher) [Kwong, supra; Stahl, supra]. T-biAbs in scFv-Fc format. JML-1/V9 and RC-1/V9 scFv-Fc were expressed in 293P cells by co-transfecting the Siglec-6 targeting scFv-Fc-_{knobs} and V9 scFv-Fc-ₕₒₗₑₛ pCEP4 plasmids and purified via Protein A affinity chromatography followed by size exclusion chromatography (SEC) with a Superdex 200 increase 10/300 GL column in conjunction with an ÄKTA FPLC instrument (all from Cytiva) [Qi 2018, supra]. T-biAbs in DART-Fc format. All DART-Fc constructs were expressed in 293Expi cells (Thermo Fisher) and purified using a 2-step Protein A affinity chromatography process with a MabSelect SuRe (Cytiva) column followed by a POROS MabCapture A (Thermo Fisher) with a pH 6 to pH 3 gradient elution in 50 mM sodium acetate, 500 mM CaCl₂) to resolve Fc/Fc-star heterodimers [Tustian A D, et al. mAbs 2016:8(4):828-38].

Human Fab library selection: The Fab-phage library from a CLL patient who had received an alloHSCT transplant was described previously [Baskar, supra]. The phage were reamplified and selected on plate-coated recombinant human Siglec-6 (Fc-Siglec-6 and Siglec-6-Fc) using established protocols, with minor adaptations including the use of the ER2378 strain (Lucigen) of Escherichia coli for library amplification and 2% non-fat dry milk for blocking [Rader C. Methods Mol Biol 2012; 901:53-79; Rader C. Methods Mol Biol 2012, 901:81-99]. Output Fab-phage screening was also conducted according to the established protocols mentioned above.

ELISA: For Fab specificity enzyme-linked immunosorbent assays (ELISAs), 50 ng of each siglec-Fc construct was coated directly into 96-well plates, blocked with 3% BSA, and incubated with 75 ng of the Fab of interest. Binding was detected with peroxidase-conjugated goat anti-human IgG, F(ab′)₂-specific secondary antibody (Jackson ImmunoResearch) and ABTS One Component HRP Substrate BioFX). Signal was quantified at 405 nm and 570 nm using a SpectraMax M5 instrument with SoftMax Pro software (Molecular Devices). For cytokine ELISAs, culture supernatants from overnight cytotoxicity assays were diluted and assayed using ELISA MAX Sets (BioLegend) to determine levels of IFN-γ, IL-2, and TNF-α. Cytokine levels were normalized to those of the positive control T-biAb CD19/V9 scFv-Fc at the highest concentration in each experiment.

SPR: Surface plasmon resonance (SPR) studies were carried out on a Biacore X100 instrument (Cytiva, formerly GE Healthcare Life Sciences) as previously described [Peng H, et al. J Mol Biol 2017429(19):2954-73]. Fab kinetics were determined by capturing Siglec-6-Fc (5-10 μg/mL) in HBS-EP+ buffer (Cytiva) and injecting titrated. For epitope binning, lower amount of antigen (0.5 pg/mL) was used along with saturating amounts of Fab (10 μM). T-biAb kinetics were determined by capturing Fc-containing T-biAbs and injecting soluble Siglec-6 (aa 28-235-His6) or human CD3ε/δ dimer (ACROBiosystems). Biacore evaluation software was used to calculate kₒₙ, k_{off}, and K_{d}.

### Homology Modeling of Siglec-6

A homology model of Siglec-6 (aa 27-236) was generated using Phyre2 [Kelley L A, et al. Nature Protocols 2015; 10(6):845-58], and CD33 (71% identity), the most similar protein with a published structure, was selected as the template for modeling. PROCHECK was employed to assess model quality [Laskowski R A, et al. J Appl Crystallogr 1993:26(2):283-91], and automated optimization was performed using YASARA [Krieger E, Vriend G. J Comput Chem 2015; 36(13):996-1007].

### Hydrogen-Deuterium Exchange (HDX) Detected by Mass Spectrometry (MS)

Solution-phase amide HDX experiments were carried out with a fully automated system (CTC HTS PAL, LEAP Technologies, Carrboro, NC: housed inside a 4° C. cabinet) as described [Chalmers M J, et al. Anal Chem 2006; 78(4):1005-14] with slight modifications. Peptides were identified using tandem MS (MS/MS) experiments performed on a QExactive (Thermo Fisher) over a 70 min gradient. Product ion spectra were acquired in a data-dependent mode and the rive most abundant ions were selected for the product ion analysis per scan event. The MS/MS *.raw data files were converted to *.mgf files and then submitted to MASCOT (version 2.3 Matrix Science, London, UK) for peptide identification. The maximum number of missed cleavages was set at 4 with the mass tolerance for precursor ions±0.6 Da and for fragment ions±8 ppm. Oxidation to methionine was selected for variable modification. Pepsin was used for digestion and no specific enzyme was selected in MASCOT during the search. Peptides included in the peptide set used for HDX detection had a MASCOT score of 20 or greater. The MS/MS MASCOT search was also performed against a decoy (reverse) sequence and false positives were ruled out if they did not pass a 1% false discovery rate.

10 μM Siglec-6 was preincubated with RC-1 Fab at a 1:1 molar ratio for 1 h on ice for complex formation before subjecting them to HDX analysis. For the differential HDX experiments. 5 μL either Siglec-6 (Apo) or the complex (1:1 molar mixture Siglec-6 and RC-1 Fab) were mixed with 20 μL of D20-containing HDX buffer (10 mM sodium phosphate, 150 mM NaCl, pH 7.8) and incubated at 4° C. for 0, 10, 30, 60, 900 or 3,600 s. Following on-exchange, unwanted forward- or back-exchange was minimized, and the protein was denatured by the addition of 25 μL of a quench solution (3 M urea, 1% TFA, pH 2.5). Samples were then immediately passed through an immobilized pepsin column (2 mm×2 cm, prepared in house) at 200 μL min⁻¹ (0.1% v/v TFA, 4° C.) and the resulting peptides were trapped and desalted on a 2 mm×10 mm C₈ trap column (Hypersil Gold, Thermo Fisher). The bound peptides were then gradient-eluted (4-40% CH₃CN v/v and 0.3% v/v formic acid) across a 2.1 mm×50 mm C₁₈ separation column (Hypersil Gold, Thermo Fisher) for 5 min. Sample handling and peptide separation were conducted at 4° C. The eluted peptides were then subjected to electrospray ionization directly coupled to a high resolution Orbitrap mass spectrometer (Exactive, Thermo Fisher).

The differential HDX experiment was performed with three technical replicates. The intensity weighted mean m/z centroid value of each peptide envelope was calculated and subsequently converted into a percentage of deuterium incorporation. This is accomplished by determining the observed averages of the undeuterated and fully deuterated spectra using the conventional formula described elsewhere [Zhang Z, Smith D L. Protein Sci 1993; 2(4):522-31]. The fully deuterated control, 100% deuterium incorporation, was calculated theoretically, and corrections for back-exchange were made based on an estimated 70% deuterium recovery and accounting for 80% final deuterium concentration in the sample (1:5 dilution in D₂O HDX buffer). Statistical significance for the differential HDX data is determined by an unpaired t-test for each time point, a procedure that is integrated into the HDX Workbench software [Pascal B D, et al. J Am Soc Mass Spectrom 2012; 23(9):1512-21].

The HDX data from all overlapping peptides were consolidated to individual amino acid values using a residue averaging approach. Briefly, for each residue, the deuterium incorporation values and peptide lengths from all overlapping peptides were assembled. A weighting function was applied in which shorter peptides were weighted more heavily and longer peptides were weighted less. Each of the weighted deuterium incorporation values were then averaged incorporating this weighting function to produce a single value for each amino acid. The initial two residues of each peptide, as well as prolines, were omitted from the calculations. This approach is similar to that previously described [Keppel T R, Weis D D. J Am Soc Mass Spectrom 2015; 26(4):547-54].

Deuterium uptake for each peptide is calculated as the average of % D for all on-exchange time points and the difference in average % D values between the unbound and bound samples is presented as a heat map in FIG. 2d, 5a, and 5c (quantification presented in FIG. 5c). In a colored heat map, peptides are colored by the software automatically to display significant differences, determined either by a >5% difference (less or more protection) in average deuterium uptake between the two states, or by using the results of unpaired t-tests at each time point (p-value <0.05 for any two time points or a p-value <0.01 for any single time point). In a colored heat map, peptides with non-significant changes between the two states are colored grey. In a colored heat map, the exchange at the first two residues for any given peptide is not colored. Each peptide bar in the heat map view displays the average A % D values, associated standard deviation, and the charge state. Additionally, overlapping peptides with a similar protection trend covering the same region are used to rule out data ambiguity.

The data have been deposited to the ProteomeXchange Consortium via the PRIDE partner repository [Perez-Riverol Y, et al. Nucleic Acids Res 2019; 47(D1):D442-D50] with the data set identifier PXD029601.

Flow cytometry: Fabs were used to stain cells at a concentration of 2 μg/mL, unless otherwise noted. Biotinylated antibody binding was detected using Alexa Fluor 647-streptavidin (Jackson ImmunoResearch) or PE-streptavidin (BD Biosciences). T-biAb was used between 0.1 and 10 μg/mL and detected using an Alexa Fluor 647-goat anti-human IgG1-Fc specific polyclonal antibody (Jackson ImmunoResearch). Commercial mAbs used for flow cytometry targeting Siglec-6 (R&D Systems, 767329), CD5 (BioLegend, L17F12), CD20 (BD Biosciences, L27), CD3 (BioLegend, OKT3), CD4 (BD, RPA-T4), CD8 (BD Biosciences. HIT8a), CD69 (BioLegend, FN50), and CD25 (BioLegend, BC96) were purchased and used at the recommended dilutions. Samples were analyzed on either an Accuri C6 Plus or a LSRII flow cytometer (both from BD Biosciences).

Cell lines: MEC1 (DSMZ, ACC 497), U937 (ATCC, CRL-1593.2), CII (DSMZ, ACC 773). HG-3 (DSMZ, ACC 765), Jurkat-Lucia NFAT (InvivoGen, jktl-nfat), FreeStyle 293-F (Thermo Fisher, R79007), HEK 293 Phoenix-AMPHO cells (293P, ATCC), HEK293S GnTi⁻ cells (ATCC, CRL-3022), and Expi293F (Thermo Fisher, A14527) cell lines were obtained from their respective commercial sources. Cell line MEC1-002 was derived from cell line MEC1 by FACS, (Chang, supra). The OSU-CLL cell line was provided by Dr. Natarajan Muthusamy (Ohio State University) under a Material Transfer Agreement (MTA) and Institutional Review Board (IRB) approval (Hertlein et al., PLoS One 8, e76607, 2013). The MDA-BM5 cell line was provided by Dr. Ian McNiece (Kellner et al., Leuk. Res. 40, 54-59, 2016). Firefly luciferase (fLuc)-expressing cells were generated by lentiviral transduction with the epHIV7 vector (Peng, supra). Transgenic cells stably expressing human Siglec-6 were generated using a hyperactive piggyBac transposase system, [Yusa K, et al. Proc Nat Acad Sci USA 2011; 108(4):1531-6] which was electroporated into CLL cell lines and sorted to generate clonal cell lines. MEC1, MEC1-002, U937, and CII cell lines were cultured in RPMI 1640 (Thermo Fisher Scientific, 11875) supplemented with 10% v/v heat-inactivated fetal bovine serum (hiFBS) (BioFluid Technologies) and 100 U/mL penicillin-streptomycin (pen-strep) and kept at 37° C. with 5% CO₂. OSU-CLL cells were cultured similarly but with 20% hiFBS. MDA-BM5 cells were cultured in MEM alpha (Thermo Fisher, 32571) with 20% hiFBS and 100 U/mL pen-strep.

Clinical samples: Cryopreserved CLL PBMC were derived from treatment-naïve patients enrolled in an observational study at the NTH Clinical Center (www.clinicaltrials.gov #NCT00923507). Cryopreserved BTKi-treated samples were obtained from CLL patients enrolled in phase 2 clinical trials at the NTH Clinical Center investigating single agent ibrutinib (NCT01500733) or acalabrutinib (NCT02337829). The studies were approved by the IRBs of the NIH Clinical Center (#08-H-0105, #12-H-0035, and #15-H-0016) and The Scripps Research Institute (#18-7179) and were in accordance with the Declaration of Helsinki. All patients gave written informed consent to participate in the study before taking part.

T cell expansion: Human T cells were expanded from healthy donor PBMC (AllCells and ZenBio) by seeding at 1×10⁶ cells/mL at a 1:1 ratio with CD3/CD28 Dynabeads (Thermo Fischer) in the presence of 100 U/mL IL-2 (Cell Sciences) over a 7-10 d culture.

NFAT activation assays: MEC1 or MEC1-002 cells (2×10⁵ cells) were seeded in 96-well round-bottom plates, and T-biAb or DPBS (vehicle) was added followed by a 30-min incubation at 37° C. Jurkat cells engineered to express luciferase under the transcriptional control of NFAT (Jurkat-Lucia NFAT, InvivoGen) were added at an effector-to-target (E:T) ratio of (2×10⁵ cells). After overnight culture, 20 μL of supernatant was combined with 50 μL of QUANTI-Luc substrate, and luminescence was recorded after 15 min using a SpectraMax M5 plate reader. The positive control for NFAT activation consisted of Jurkat-Lucia NFAT cells plated alone with 0.5 mg/mL concanavalin A (conA). Percent NFAT activation was calculated as follows:

$$NFAT activation ( \% ) = \frac{\text{?}}{\text{?}} * 100$$

$$\text{?} \text{indicates text missing or illegible when filed}$$

In vitro cytotoxicity assays: Cell line cytotoxicity experiments were modeled off a previously published luciferase assay format (22Brown et al., J. Immunol. Methods 297, 39-52, 2005). MEC1-fLuc, MEC1-002 fLuc or MEC1-fLuc-hS6 were seeded at 2.5′ 10° cells per well along with T-biAb or DPBS (vehicle) followed by a 30-min incubation at 37° C. T cells expanded from healthy donor PBMC [Qi 2018, supra] were added at a 1:1 ratio and cells were co-cultured for 16-24 h. Luciferin (Biosynth Carbosynth) was added to 0.15 mg/mL and luminescence was recorded after 15 min as above. Luminescent signal from T cell and T-biAb treated wells was normalized to the positive viability control consisting of cells only. Specific lysis was calculated as follows:

$$Specific lysis ( \% ) = ( 1 - \frac{\text{?}}{\text{?}} ) * 100$$

$$\text{?} \text{indicates text missing or illegible when filed}$$

Ex vivo activity assays: T-biAb activity on cryopreserved CLL PBMC was evaluated as previously described [Robinson, supra]. Cell viability of the CLL population (CD3⁻/CD5⁺/CD20⁺) was assessed at various time points with the LIVE/DEAD fixable violet stain (Thermo Fisher) by flow cytometry. Patient samples in which the vehicle-treated cells did not meet the viability threshold (>20%) at a given time point were eliminated from analysis. T cell populations were quantified with the aforementioned commercial mAbs to CD4, CD8, CD25, and CD69.

Multispectral fluorescence imaging: MEC1-hS6 cells were stained with CellTrace Far Red dye (Thermo Fisher), followed by incubation with 5 μg/mL T-biAb for 30 min at 37° C. Excess T-biAb was washed off and Jurkat T cells, stained with CellTrace CFSE dye, were added and co-cultured (E:T=1:10) for 30 min at 37° C. to allow synapse formation to occur. Conjugates were washed, fixed with Cytofix/Cytoperm solution (BD Biosciences), stained with Cy3-conjugated goat anti-human Fc (Jackson ImmunoResearch) and DAPI (Thermo Fisher), and imaged (˜50,000 per sample) using an Amnis ImageStream^{x} Mk II instrument (Luminex) at 40× magnification. IDEAS 6.3 software (Luminex) was used for analysis. Images were gated to identify individual DAPI⁺ Jurkat and DAPI⁺ MEC1-hS6 cells, then a mask was created for the overlap between Jurkat and MEC1-hS6 cells. Next, data were filtered on the size of the overlap mask to eliminate cells that were clearly on top of each other or too far apart for a 1:1 conjugate, and an additional gate was added to require Cy3 (T-biAb) staining at the synapse. Synapse formation was quantified as the number of heterodoublet synapse conjugates relative to the total number of DAPI Jurkat cells. An additional mask was created using the union of DAPI and cell stains, and internuclear distance was calculated between the centroids of the DAPI cell masks for each cell type using the distance theorem.

Xenograft mouse model: Six- to seven-week-old female NOD-scid IL2RTγ^{null} (NSG) mice (The Jackson Laboratory, JAX #005557) were inoculated with 2×10⁶ MEC1-fLuc-hS6 cells i.v. (tail vein). One day prior to each treatment, mice were randomized and pre-conditioned with 0.25 mL human serum by i.p. injection. Mice were treated on day 7 with 3×10⁶ human T cells and given T-biAb (0.5 mg/kg or 0.05 mg/kg, diluted in DPBS, 5 mice per group) twice weekly for 2 weeks, all i.v. tail vein. On day 13 mice were given an additional 1×10⁶ T cells. Mice were imaged with a Lago X instrument using templated imaging protocols and consistent timing after luciferin injection and data were analyzed using Aura software (Spectral Instruments) similar to previous reports [Qi J, et al. Angew Chem Int Ed Engl 2020; 59(29):12178-85]. All procedures were approved by the Institutional Animal Care and Use Committee (IACUC #15-026) of The Scripps Research Institute (Jupiter, FL) and were performed according to the NIH Guide for the Care and Use of Laboratory Animals.

PK study: Six- to seven-week-old female NOD-scid IL2Rγ^{null} (NSG) mice (The Jackson Laboratory, JAX #005557) were pre-conditioned with 0.25 mL human serum or DPBS one day prior to treatment. Three to four mice per group were injected i.v. or i.p. with RC-1/V9 aDART-Fc at 2.5 mg/kg. Blood was collected at 5 min, 30 min, 2, 8, 24, 48, 72, 96, 144, 216, 336, and 528 h after injection maintaining treatment order with blood collection, into heparinized capillary tubes from the tail vein. Plasma was obtained by centrifuging the samples at 2,000×g for 5 min and was stored at −80° C. until analysis. The concentration of T-biAbs in the plasma samples was measured by ELISA. CD3c/o (ACROBiosystems) was coated directly onto half-area ELISA plates (200 ng/well) overnight and blocked with 3% w/v bovine serum albumin (BSA). Serum samples or T-biAb standard were added to the plate followed by 2-h incubation, and T-biAb was detected with HRP-conjugated AffiniPure goat anti-human-Fc secondary antibody (Jackson ImmunoResearch). The concentration of T-biAb in the plasma samples was interpolated from a four-parameter logistic model fit of the standard curve (GraphPad Prism). PK parameters were calculated from the time points within the linear range by using Phoenix WinNonlin PK/PD Modeling and Analysis software (Pharsight).

### Statistics

Statistical significance for the differential HDX data is determined by an unpaired t-test for each time point, a procedure that is integrated into the HDX Workbench software [Pascal, supra]. For biochemical and in vitro data, a two-tailed student's t-test was used to determine calculate statistics. To compare the effects of multiple ex vivo treatments on each patient sample. Wilcoxon matched-pairs signed rank test was used. For in vivo studies, 4 (PK study) to 5 (treatment model) mice were randomized and treated per experimental group, based on the resource equation method, as there were multiple readouts [Charan J, Kantharia N D. J Pharmacol Pharmacother 2013; 4(4):303-6]. Treatment and analysis were unblinded. Welch's t-test was used to compare bioluminescent signal among treatment groups, as standard deviation was not uniform across groups, while the Mantel-Cox log-rank analysis was used to compare survival advantages. Statistical analysis was conducted using GraphPad Prism and significance was established if p<0.05, unless otherwise noted.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. An isolated antibody that binds to the same epitope on sialic acid-binding immunoglobulin-like lectin-6 (Siglec-6) as antibody RC-1, RC-2, or ARN-1.

2. The antibody of claim 1,
comprising three heavy chain CDRs and three light chain CDRs of antibody RC-1, wherein RC-1 is characterized by a mature heavy chain variable region having an amino acid sequence comprising SEQ ID NO:1 and a mature light chain variable region having an amino acid sequence comprising SEQ ID NO:2;
comprising three heavy chain CDRs and threw light chain CDRs of antibody RC-2, wherein RC-2 is characterized by a mature heavy chain variable region having an amino acid sequence comprising SEQ ID NO:3 and a mature light chain variable region having an amino acid sequence comprising SEQ ID NO:4; or comprising three heavy chain CDRs and three light chain CDRs of antibody ARN-1, wherein ARN-1 is characterized by a mature heavy chain variable region having an amino acid sequence comprising SEQ ID NO:5 and a mature light chain variable region having an amino acid sequence comprising SEQ ID NO:6.

3. The antibody of claim 2, wherein the CDRs are of a definition selected from the group consisting of Kabat, Chothia, Kabat/Chothia Composite, AbM, Contact, and IMGT.

4. The antibody of claim 3,
wherein the mature heavy chain variable region comprises the three Kabat heavy chain CDRs of SEQ ID NOs:7-9 and the mature light chain variable region comprises the three Kabat light chain CDRs of SEQ ID NOs: 10-12;
wherein the mature heavy chain variable region comprises the three Kabat heavy chain CDRs of SEQ ID NOs:13-15 and the mature light chain variable region comprises the three Kabat light chain CDRs of SEQ ID NOs:16-18; or
wherein the mature heavy chain variable region comprises the three Kabat heavy chain CDRs of SEQ ID NOs:19-21 and the mature light chain variable region comprises the three Kabat light chain CDRs of SEQ ID NOs:22-24.

5. The antibody of any one of claims 1-4,
comprising a mature heavy chain variable region having an amino acid sequence at least 90% identical to SEQ ID NO: 1, and a mature light chain variable region having an amino acid sequence at least 95% identical to SEQ ID NO:2;
comprising a mature heavy chain variable region having an amino acid sequence at least 90% identical to SEQ ID NO:3, and a mature light chain variable region having an amino acid sequence at least 95% identical to SEQ ID NO:4; or
comprising a mature heavy chain variable region having an amino acid sequence at least 90% identical to SEQ ID NO: 5, and a mature light chain variable region having an amino acid sequence at least 90% identical to SEQ ID NO:6.

6. The antibody of claim 5,
comprising a mature heavy chain variable region having an amino acid sequence at least 95% identical to SEQ ID NO: 1, and a mature light chain variable region having an amino acid sequence at least 95% identical to SEQ ID NO:2;
comprising a mature heavy chain variable region having an amino acid sequence at least 95% identical to SEQ ID NO:3, and a mature light chain variable region having an amino acid sequence at least 95% identical to SEQ ID NO:4; or
comprising a mature heavy chain variable region having an amino acid sequence at least 95% identical to SEQ ID NO:5, and a mature light chain variable region having an amino acid sequence at least 95% identical to SEQ ID NO:6.

7. The antibody of claim 6,
comprising a mature heavy chain variable region having an amino acid sequence of SEQ ID NO: 1, and a mature light chain variable region having an amino acid sequence of SEQ ID NO:2;
comprising a mature heavy chain variable region having an amino acid sequence of SEQ ID NO:3, and a mature light chain variable region having an amino acid sequence of SEQ ID NO:4; or
comprising a mature heave chain variable region having an amino acid sequence of SEQ ID NO:5, and a mature light chain variable region having an amino acid sequence of SEQ ID NO:6.

8. The antibody of one of claims 1-7, wherein the antibody is chimeric, humanized, or human.

9. The antibody of claim 8, wherein the antibody is human.

10. The antibody of any preceding claim, wherein the antibody is IgA1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, a dual-affinity re-targeting (DART) antibody, or a bivalent antibody.

11. The antibody of any preceding claim, wherein the mature light chain variable region is fused to a light chain constant region and the mature heavy chain variable region is fused to a heavy chain constant region.

12. The antibody of claim 11, wherein the heavy chain constant region is a mutant form of a natural human heavy chain constant region which has reduced binding to a Fcγ receptor relative to the natural human heavy chain constant region.

13. The antibody of claim 1, which is a scFv.

14. The antibody of claim 13, further comprising a Fc domain that is fused to the C-terminus of the scFv.

15. The antibody of claim 14, comprising an amino acid sequence at least 90% identical to SEQ ID NO:25, wherein the mature heavy chain variable region comprises the three Kabat heavy chain CDRs of SEQ ID NOs:7-9 and the mature light chain variable region comprises the three Kabat light chain CDRs of SEQ ID NOs:10-12.

16. The antibody of any preceding claim, which is an antibody fragment.

17. The antibody of any one of claims 1-15, which is a bispecific antibody.

18. The antibody of claim 17, wherein the bispecific antibody is a T-cell engaging bispecific antibody.

19. The antibody of any one of claims 1-15, which is an antibody-based binding protein.

20. The antibody of any preceding claim, which is conjugated to a synthetic molecule.

21. The antibody of claim 20, % herein the synthetic molecule is a cytotoxic agent, a label, a therapeutic radioisotope, a diagnostic radioisotope, or a liposome.

22. The antibody of claim 21, wherein the cytotoxic agent is a small molecule weight toxin, a peptide toxin, or a protein toxin.

23. The antibody of any one of claims 1-19 linked to at least one cytotoxic agent as an antibody drug conjugate.

24. The antibody of claim 23, wherein the cytotoxic agent is a small molecular weight toxin, a peptide toxin, or a protein toxin, or a radionuclide.

25. AT cell-engaging bispecific antibody (T-biAb), comprising (1) a V_{H} domain and a V_{L} domain of a T cell targeting antibody, and (2) a V_{H} domain and a V_{L} domain of an antibody of claim 1 (Siglec-6 targeting antibody);
wherein the V_{H} domain of the Siglec-6 targeting antibody comprises the three Kabat heavy chain CDRs of SEQ ID NOs:7-9 and the V_{L} domain of the Siglec-6 targeting antibody comprises the three Kabat light chain CDRs of SEQ ID NOs:10-12;
wherein the V_{H} domain of the Siglec-6 targeting antibody comprises the three Kabat heavy chain CDRs of SEQ ID NOs: 13-15 and the V_{L} domain of the Siglec-6 targeting antibody comprises the three Kabat light chain CDRs of SEQ ID NOs:16-18; or
wherein the V_{H} domain of the Siglec-6 targeting antibody comprises the three Kabat heavy chain CDRs of SEQ ID NOs:19-21 and the V_{L} domain of the Siglec-6 targeting antibody comprises the three Kabat light chain CDRs of SEQ ID NOs:22-24.

26. The T-biAb of claim 25, wherein the T cell targeting antibody is an anti-CD3 antibody.

27. The T-biAb of claim 26, wherein the T cell targeting antibody is an anti-CD3 antibody comprising a heavy variable region having an amino acid sequence of SEQ ID NO:34 and light variable region having an amino acid sequence of SEQ ID NO:35.

28. The T-biAb of claim 26, wherein the T cell targeting antibody is an anti-CD3 antibody comprising an amino acid sequence of SEQ ID NO:33.

29. The T-biAb of claim 26, wherein the T cell targeting antibody is an anti-CD3 antibody comprising an amino acid sequence of SEQ ID NO:39.

30. The T-biAb of claim 25, wherein the V_{H} domain and the V_{L} domain of the T cell targeting antibody forms a first scFv, and the V_{H} domain and the V_{L} domain of the Siglec-6 targeting antibody forms a second scFv.

31. The T-biAb of claim 25, wherein the V_{H} domain of the T cell targeting antibody and the V_{L} domain of the Siglec-6 targeting antibody forms a first tandem V_{H}-V_{L} fragment, and the V_{L} domain of the T cell targeting antibody and the V_{H} domain of the Siglec-6 targeting antibody forms a second tandem V_{H}-V_{L} fragment.

32. The T-biAb of claim 31, further comprising a C-terminal inter-chain disulfide bond to connect the two tandem V_{H}-V_{L} fragments.

33. The T-biAb of claim 31, wherein one or both tandem V_{H}-V_{L} fragments are further fused to an antibody Fc arm in either a V_{H}-V_{L}-Fc format or a V_{L} V_{H}-Fc format.

34. The T-biAb of claim 33, comprising two polypeptide chains each of which comprises a tandem V_{H}-V_{L} fragment that is fused to a Fc arm, wherein the two Fc arms respectively contain knob mutations and hole mutations to allow dimerization of the two polypeptide chains.

35. The T-biAb of claim 34, wherein the first polypeptide chain comprises a tandem V_{H}-V_{L} fragment of a T cell targeting V_{L} and a Siglec-6 targeting V_{H} that is fused to one Fc arm, and the second polypeptide chain comprises a tandem V_{H}-V_{L} fragment of a Siglec-6 targeting V_{L} and a T cell targeting V_{H} that is fused to the other Fc arm.

36. The T-biAb of claim 35, where (a) the first polypeptide chain comprises an amino acid sequence that is at least 90% identical to SEQ ID NO:26, and a Siglec-6 targeting V_{H} domain comprising the three Kabat heavy chain CDRs of SEQ ID NOs:7-9; and (b) the second polypeptide chain comprises an amino acid sequence that is at least 90% identical to SEQ ID NO:27, and a Siglec-6 targeting V_{L} domain comprising the three Kabat light chain CDRs of SEQ ID NOs:10-12.

37. The T-biAb of claim 34, wherein the first polypeptide chain comprises a tandem V_{H}-V_{L} fragment of a Siglec-6 targeting V_{L} and a T cell targeting V_{H} that is fused to one Fc arm, and the second polypeptide chain comprises a tandem V_{H}-V_{L} fragment of a T cell targeting V_{L} and a Siglec-6 targeting V_{H} that is fused to the other Fc arm.

38. The T-biAb of claim 37, where (a) the first polypeptide chain comprises an amino acid sequence that is at least 90% identical to SEQ ID NO:28, and a Siglec-6 targeting V_{L} domain comprising the three Kabat light chain CDRs of SEQ ID NOs:10-12; and (b) the second polypeptide chain comprises an amino acid sequence that is at least 90% identical to SEQ ID NO:29, and a Siglec-6 targeting V_{H} domain comprising the three Kabat heavy chain CDRs of SEQ ID NOs:7-9.

39. The T-biAb of claim 33, comprising three polypeptide chains, wherein (a) the first polypeptide chain comprises a first tandem V_{H}-V_{L} fragment having a first C-terminal coil motif that is fused to a first Fc arm, (b) the second polypeptide chain comprises the other tandem V_{H}-V_{L} fragment having a second C-terminal coil motif, and (c) the third polypeptide chain comprises a second Fc arm; wherein one of the Fc arms contains knob mutations, and the other Fc arm contains hole mutations; and wherein one of the C-terminal coil motif is a E-coil, and the other C-terminal coil motif is an oppositely charged K-coil.

40. The T-biAb of claim 39, wherein (a) the first tandem V_{H}-V_{L} fragment comprises a Siglec-6 targeting V_{L} and a T cell targeting V_{H}, and the second tandem V_{H}-V_{L} fragment comprises a T cell targeting V_{L} and a Siglec-6 targeting V_{H}, or (b) the first tandem V_{H}-V_{L} fragment comprises a T cell targeting V_{L} and a Siglec-6 targeting V_{H}, and the second tandem V_{H}-V_{L} fragment comprises a Siglec-6 targeting V_{L} and a T cell targeting V_{H}.

41. The T-biAb of claim 40, wherein (a) the first polypeptide chain comprises (i) a Siglec-6 targeting V_{L} domain comprising the three Kabat light chain CDRs of SEQ ID NOs:10-12 and (ii) an overall amino acid sequence that is at least 90% identical to SEQ ID NO:30; (b) the second polypeptide chain comprises (i) a Siglec-6 targeting V_{H} domain comprising the three Kabat heavy chain CDRs of SEQ ID NOs:7-9 and (ii) an overall amino acid sequence that is at least 90% identical to SEQ ID NO:31, and (c) the third polypeptide chain comprises an amino acid sequence that is at least 90% identical to SEQ ID NO:32.

42. A chimeric antigen receptor (CAR), comprising an extracellular domain, a transmembrane domain, and an intracellular signaling domain, wherein the extracellular domain comprises the antibody of any one of claims 1-7.

43. An effector cell expressing the chimeric antigen receptor of claim 42.

44. The effector cell of claim 43, which is a T cell or a natural killer (NK) cell.

45. A pharmaceutical composition comprising a therapeutically effective amount of the antibody of any one of claims 1-24, or the T cell-engaging bispecific antibody of any one of claims 25-41 and a pharmaceutically acceptable carrier.

46. A polynucleotide encoding a heavy chain variable region and/or a light chain variable region of the antibody of any one of claims 1-24.

47. A vector comprising the polynucleotide of claim 46.

48. A host cell harboring the vector of claim 47.

49. A method of treating a disease or disorder associated with aberrant Siglec-6 expression in a subject, comprising administering the pharmaceutical composition of claim 45 to a subject in need thereof.

50. The method of claim 49, wherein the disease or disorder is a tumor, a leukemia, or a mast cell disorder.

51. The method of claim 49, wherein the disease or disorder is a chronic or an acute leukemia.

52. The method of claim 49, wherein the disease or disorder is chronic lymphocytic leukemia (CLL).

53. The method of claim 49, wherein the disease or disorder is a tumor containing Siglec-6 expressing myeloid-derived suppressor cells (MDSCs).

54. The method of claim 49, wherein the disease or disorder is an autoimmune disease or an inflammatory disease.

55. The method of claim 54, wherein the autoimmune disease or the inflammatory disease is multiple sclerosis, rheumatoid arthritis, systemic lupus erythematosus, type 2 diabetes mellitus, cytokine release syndrome, graft-versus-host-disease, or HIV-associated immunopathogenesis.


SEQUENCE LISTING (48 sequences)

SEQ ID NO: 1 - protein, 114 aa, Homo sapiens
EVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCANYGMDVWGKGTTVTVSS

SEQ ID NO: 2 - protein, 107 aa, Homo sapiens
AIRLTQSPSSLSASVGDRVTITCRASQSISSYLNWYQQKPGKAPKLLIYAASSLQSGVPSRFSGSGSGTDFTLTISSLQPEDFATYYCQQSYGTPFTFGPGTKVDIK

SEQ ID NO: 3 - protein, 116 aa, Homo sapiens
EVQLLESGGGVVQPGRSLRLSCAASGFTFSSYGMHWVRQAPGKGLEWVAVISYDGSNKHYADSVKGRFTISRDNSKNTLYLQMNSLRAEDTAVYYCARGGQTIDIWGQGTMVTVSS

SEQ ID NO: 4 - protein, 107 aa, Homo sapiens
DIVMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 5 - protein, 116 aa, Homo sapiens
EVQLVQSGGGVVQPGRSLTLSCAAFGFTFSTYTMHWVRQAPGKGLEWVAVISYDEGFKYYADSVKGRFTISRDISKNTLYLQMNSLRTEDTAVYYCAREASAFDIWGQGTMVTVSS

SEQ ID NO: 6 - protein, 107 aa, Homo sapiens
SYVLTQPPLVSVSPGQTARITCSGENWGQKYASWYQQKPGQSPMLIIYEDTKRPSEIPERFSGSNSGSTVTLTISGTQPMDEADYYCQAWDDSTGGVFGTGTKVTVL

SEQ ID NO: 7 - protein, 5 aa, Homo sapiens
SYGMH

SEQ ID NO: 8 - protein, 17 aa, Homo sapiens
VISYDGSNKYYADSVKG

SEQ ID NO: 9 - protein, 5 aa, Homo sapiens
YGMDV

SEQ ID NO: 10 - protein, 11 aa, Homo sapiens
RASQSISSYLN

SEQ ID NO: 11 - protein, 7 aa, Homo sapiens
AASSLQS

SEQ ID NO: 12 - protein, 9 aa, Homo sapiens
QQSYGTPFT

SEQ ID NO: 13 - protein, 5 aa, Homo sapiens
SYGMH

SEQ ID NO: 14 - protein, 17 aa, Homo sapiens
VISYDGSNKHYADSVKG

SEQ ID NO: 15 - protein, 7 aa, Homo sapiens
GGQTIDI

SEQ ID NO: 16 - protein, 11 aa, Homo sapiens
RASQSISSYLN

SEQ ID NO: 17 - protein, 7 aa, Homo sapiens
AASSLQS

SEQ ID NO: 18 - protein, 9 aa, Homo sapiens
QQSYSTPYT

SEQ ID NO: 19 - protein, 5 aa, Homo sapiens
TYTMH

SEQ ID NO: 20 - protein, 17 aa, Homo sapiens
VISYDEGFKYYADSVKG

SEQ ID NO: 21 - protein, 7 aa, Homo sapiens
EASAFDI

SEQ ID NO: 22 - protein, 11 aa, Homo sapiens
SGENWGQKYAS

SEQ ID NO: 23 - protein, 7 aa, Homo sapiens
EDTKRPS

SEQ ID NO: 24 - protein, 10 aa, Homo sapiens
QAWDDSTGGV

SEQ ID NO: 25 - protein, 468 aa, Synthetic construct
EVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCANYGMDVWGKGTTVTVSSGGGGSGGGGSGGGGSAIRLTQSPSSLSASVGDRVTITCRASQSISSYLNWYQQKPGKAPKLLIYAASSLQSGVPSRFSGSGSGTDFTLTISSLQPEDFATYYCQQSYGTPFTFGPGTKVDIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 26 - protein, 452 aa, Synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGSGGGGEVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCANYGMDVWGKGTTVTVSSGCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGA

SEQ ID NO: 27 - protein, 461 aa, Synthetic construct
AIRLTQSPSSLSASVGDRVTITCRASQSISSYLNWYQQKPGKAPKLLIYAASSLQSGVPSRFSGSGSGTDFTLTISSLQPEDFATYYCQQSYGTPFTFGPGTKVDIKGGGGSGGGGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 28 - protein, 460 aa, Synthetic construct
AIRLTQSPSSLSASVGDRVTITCRASQSISSYLNWYQQKPGKAPKLLIYAASSLQSGVPSRFSGSGSGTDFTLTISSLQPEDFATYYCQQSYGTPFTFGPGTKVDIKGGGSGGGGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVFDSDGSFFLVSKLTVDKSRWQQGNVFSCSVMHEALHNRFTQKSLSLSPGA

SEQ ID NO: 29 - protein, 383 aa, Synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGEVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCANYGMDVWGKGTTVTVSSGCPPCPAPEAAGGPSVFLFPPKPKDTLMISRTPEVTCVVVDVSHEDPEVKFNWYVDGVEVHNAKTKPRVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 30 - protein, 500 aa, Synthetic construct
AIRLTQSPSSLSASVGDRVTITCRASQSISSYLNWYQQKPGKAPKLLIYAASSLQSGVPSRFSGSGSGTDFTLTISSLQPEDFATYYCQQSYGTPFTFGPGTKVDIKGGGSGGGGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 31 - protein, 263 aa, Synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGEVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCANYGMDVWGKGTTVTVSSASTKGKVAACKEKVAALKEKVAALKEKVAALKE

SEQ ID NO: 32 - protein, 227 aa, Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGA

SEQ ID NO: 33 - protein, 244 aa, Synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 34 - protein, 122 aa, Synthetic construct [sequence omitted]

SEQ ID NO: 35 - protein, 107 aa, Synthetic construct [sequence omitted]

SEQ ID NO: 36 - protein, 15 aa, Synthetic construct
GGGGSGGGGSGGGGS

SEQ ID NO: 37 - protein, 220 aa, Synthetic construct
  REGION 1..3: note = N-terminal expression tag
  REGION 212..220: note = C-terminal expression tag
ETGERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSGTKHHHHHH

SEQ ID NO: 38 - protein, 470 aa, Synthetic construct
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 39 - protein, 476 aa, Synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGA

SEQ ID NO: 40 - protein, 9 aa, Homo sapiens
ARGGQTIDI

SEQ ID NO: 41 - protein, 9 aa, Homo sapiens
QQSYSTPFT

SEQ ID NO: 42 - protein, 7 aa, Homo sapiens
ANYGMDV

SEQ ID NO: 43 - protein, 9 aa, Homo sapiens
AREASAFDI

SEQ ID NO: 44 - protein, 20 aa, Homo sapiens
FFRLKSKWMKYGYTSSKLSV

SEQ ID NO: 45 - protein, 17 aa, Homo sapiens
RVMALTHRPNISIPGTL

SEQ ID NO: 46 - protein, 209 aa, Homo sapiens
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVS

SEQ ID NO: 47 - protein, 211 aa, Macaca mulatta
QEQRFRLEGPESLTVQEGLCVLLPCRLPTIYSPWYSYYGYWFLEGADVPVATNDPDEEVQEETRGRFHLLWDLSSKNCSLSIRDARRRDTAVYFFQLKRKGTSGYTSYVSSKLSVCVMALTHRPSISIPGTLESGCSRTLTCSVPWACEQGTPPIFSWMSAAPTSLGLRTTQSSVLTITPRPQDHSTNLTCQVMYPGVGVTMERTIQLNVS

SEQ ID NO: 48 - protein, 212 aa, Homo sapiens
FWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVT